(19) United States
(12) Patent Application Publication
(10) Pub. No.: US 2014/0148442 A1
(43) Pub. Date: May 29, 2014

(54) FAST-DISSOLVE DOSAGE FORMS OF 5-HT2C AGONISTS

(71) Applicants: Anthony C. Blackburn, San Diego, CA (US); Scott Stirn, San Diego, CA (US); Yun Shan, San Diego, CA (US); Anna Shifrina, San Diego, CA (US)

(72) Inventors: Anthony C. Blackburn, San Diego, CA (US); Scott Stirn, San Diego, CA (US); Yun Shan, San Diego, CA (US); Anna Shifrina, San Diego, CA (US)

(73) Assignee: Arena Pharmaceuticals, Inc.

(21) Appl. No.: 13/820,123

(22) PCT Filed: Aug. 31, 2011
(86) PCT No.: PCT/US11/49953

§ 371 Date: Aug. 8, 2013

Related U.S. Application Data
(60) Provisional application No. 61/402,589, filed on Sep. 1, 2010.

(51) Int. Cl. C07D 223/16; A61K 45/06; A61K 31/55

(52) U.S. Cl. CPC C07D 223/16; A61K 31/55; A61K 45/06
USPC 514/217.01; 540/594

(57) ABSTRACT

Salts of the 5-HT_{2C}-receptor agonist (R)-8-chloro-1-methyl-2,3,4,5-tetrahydro-1H-3-benzazepine, and dosage forms comprising them that are useful for, inter alia, weight management.

### FIELD OF THE INVENTION

The present invention relates to salts of the 5-HT_{2C}-receptor agonist (R)-8-chloro-1-methyl-2,3,4,5-tetrahydro-1H-3-benzazepine, and dosage forms comprising them that are useful for, inter alia, weight management.

### BACKGROUND OF THE INVENTION

Obesity is a life-threatening disorder in which there is an increased risk of morbidity and mortality arising from concomitant diseases such as type II diabetes, hypertension, stroke, cancer and gallbladder disease.

Obesity is now a major healthcare issue in the Western World and increasingly in some third world countries. The increase in numbers of obese people is due largely to the increasing preference for high fat content foods but also the decrease in activity in most people's lives. Currently about 30% of the population of the USA is now considered obese.

Whether someone is classified as overweight or obese is generally determined on the basis of their body mass index (BMI) which is calculated by dividing body weight (kg) by height squared (m2). Thus, the units of BMI are kg/m² and it is possible to calculate the BMI range associated with minimum mortality in each decade of life. Overweight is defined as a BMI in the range 25-30 kg/m², and obesity as a BMI greater than 30 kg/m² (see table below).

Classification Of Weight By Body Mass Index (BMI)

| BMI | CLASSIFICATION |
|---|---|
| <18.5 | Underweight |
| 18.5-24.9 | Normal |
| 25.0-29.9 | Overweight |
| 30.0-34.9 | Obesity (Class I) |
| 35.0-39.9 | Obesity (Class II) |
| >40 | Extreme Obesity (Class III) |

As the BMI increases there is an increased risk of death from a variety of causes that are independent of other risk factors. The most common diseases associated with obesity are cardiovascular disease (particularly hypertension), diabetes (obesity aggravates the development of diabetes), gall bladder disease (particularly cancer) and diseases of reproduction. The strength of the link between obesity and specific conditions varies. One of the strongest is the link with type 2 diabetes. Excess body fat underlies 64% of cases of diabetes in men and 77% of cases in women (Seidell, Semin Vasc Med, 5:3-14 (2005)). Research has shown that even a modest reduction in body weight can correspond to a significant reduction in the risk of developing coronary heart disease.

There are problems however with the BMI definition in that it does not take into account the proportion of body mass that is muscle in relation to fat (adipose tissue). To account for this, obesity can also be defined on the basis of body fat content: greater than 25% in males and greater than 30% in females.

Obesity considerably increases the risk of developing cardiovascular diseases as well. Coronary insufficiency, atheromatous disease, and cardiac insufficiency are at the forefront of the cardiovascular complications induced by obesity. It is estimated that if the entire population had an ideal weight, the risk of coronary insufficiency would decrease by 25% and the risk of cardiac insufficiency and of cerebral vascular accidents would decrease by 35%. The incidence of coronary diseases is doubled in subjects less than 50 years of age who are 30% overweight. The diabetes patient faces a 30% reduced lifespan. After age 45, people with diabetes are about three times more likely than people without diabetes to have significant heart disease and up to five times more likely to have a stroke. These findings emphasize the inter-relations between risks factors for diabetes and coronary heart disease and the potential value of an integrated approach to the prevention of these conditions based on the prevention of obesity (Perry, I. J., et al., BMJ 310, 560-564 (1995)).

Diabetes has also been implicated in the development of kidney disease, eye diseases and nervous system problems. Kidney disease, also called nephropathy, occurs when the kidney's “filter mechanism” is damaged and protein leaks into urine in excessive amounts and eventually the kidney fails. Diabetes is also a leading cause of damage to the retina at the back of the eye and increases risk of cataracts and glaucoma. Finally, diabetes is associated with nerve damage, especially in the legs and feet, which interferes with the ability to sense pain and contributes to serious infections. Taken together, diabetes complications are one of the nation's leading causes of death.

The first line of treatment is to offer diet and life style advice to patients such as reducing the fat content of their diet and increasing their physical activity. However, many patients find this difficult and need additional help from drug therapy to maintain results from these efforts.

Most currently marketed products have been unsuccessful as treatments for obesity because of a lack of efficacy or unacceptable side-effect profiles. The most successful drug so far was the indirectly acting 5-hydroxytryptamine (5-HT) agonist d-fenfluramine (Redux™) but reports of cardiac valve defects in up to one third of patients led to its withdrawal by the FDA in 1998.

In addition, two drugs have been launched in the USA and Europe: Orlistat (Xenical™) a drug that prevents absorption of fat by the inhibition of pancreatic lipase, and Sibutramine (Reductil™), a 5-HT/noradrenaline re-uptake inhibitor. However, side effects associated with these products may limit their long-term utility. Treatment with Xenical™ is reported to induce gastrointestinal distress in some patients, while Sibutramine has been associated with raised blood pressure in some patients.

Serotonin (5-HT) neurotransmission plays an important role in numerous physiological processes both in physical and in psychiatric disorders. 5-HT has been implicated in the regulation of feeding behavior. 5-HT is believed to work by inducing a feeling of satiety, such that a subject with enhanced 5-HT stops eating earlier and fewer calories are consumed. It has been shown that a stimulatory action of 5-HT on the 5-HT_{2C} receptor plays an important role in the control of eating and in the anti-obesity effect of d-fenfluramine. As the 5-HT_{2C} receptor is expressed in high density in the brain (notably in the limbic structures, extrapyramidal pathways, thalamus and hypothalamus i.e. PVN and DMH, and predominantly in the choroid plexus) and is expressed in low density or is absent in peripheral tissues, a selective 5-HT_{2C} receptor agonist can be a more effective and safe anti-obesity agent. Also, 5-HT_{2C} knockout mice are overweight with cognitive impairment and susceptibility to seizure.

It is believed that the 5-HT_{2C} receptor may play a role in obsessive compulsive disorder, some forms of depression, and epilepsy. Accordingly, agonists can have anti-panic properties, and properties useful for the treatment of sexual dysfunction.

In sum, the 5-HT_{2C} receptor is a receptor target for the treatment of obesity and psychiatric disorders, and it can be seen that there is a need for selective 5-HT_{2C} agonists which safely decrease food intake and body weight.

The salts and formulations of the present invention comprise the selective 5-HT_{2C}-receptor agonist (R)-8-chloro-1-methyl-2,3,4,5-tetrahydro-1H-3-benzazepine (Compound 1), and are useful for, inter alia, weight management, including weight loss and the maintenance of weight loss. Compound 1 is disclosed in PCT patent publication WO2003/086303, which is incorporated herein by reference in its entirety.

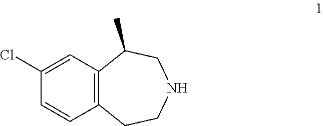

Various synthetic routes to (R)-8-chloro-1-methyl-2,3,4,5-tetrahydro-1H-3-benzazepine, its related salts, enantiomers, crystalline forms, and intermediates, have been reported in PCT publications, WO 2005/019179, WO 2006/069363, WO 2007/120517, WO 2008/070111, WO 2009/111004, and in U.S. provisional application 61/396,752 each of which is incorporated herein by reference in its entirety.

Combinations of (R)-8-Chloro-1-methyl-2,3,4,5-tetrahydro-1H-3-benzazepine with other agents, including without limitation, phentermine, and uses of such combinations in therapy are described in WO 2006/071740, which is incorporated herein by reference in its entirety.

The following United States provisional applications are related to (R)-8-chloro-1-methyl-2,3,4,5-tetrahydro-1H-3-benzazepine: 61/402,578; 61/403,143; 61/402,580; 61/402,628; 61/403,149; 61/402,589; 61/402,611; 61/402,565; 61/403,185; each of which is incorporated herein by reference in its entirety.

The following applications are related to (R)-8-chloro-1-methyl-2,3,4,5-tetrahydro-1H-3-benzazepine and have the same filing date as the subject application: Attorney Reference Number 178.WO1, a PCT application which claims priority to U.S. provisional applications 61/402,578 and 61/403,143; Attorney Reference Number 181.WO1, a PCT application which claims priority to U.S. provisional application 61/402,580; Attorney Reference Number 186.WO1, a PCT application which claims priority to U.S. provisional applications 61/402,628 and 61/403,149; Attorney Reference Number 188.WO1, a PCT application which claims priority to U.S. provisional application 61/402,611; and Attorney Reference Number 192.WO1, a PCT application which claims priority to U.S. provisional applications 61/402,565 and 61/403,185; each of which is incorporated herein by reference in its entirety.

(R)-8-Chloro-1-methyl-2,3,4,5-tetrahydro-1H-3-benzazepine hydrochloride (lorcaserin hydrochloride) is an agonist of the 5-HT_{2C} receptor and shows effectiveness at reducing obesity in animal models and humans. In December 2009, Arena Pharmaceuticals submitted a New Drug Application, or NDA, for lorcaserin to the FDA. The NDA submission is based on an extensive data package from lorcaserin's clinical development program that includes 18 clinical trials totaling 8,576 patients. The pivotal phase 3 clinical trial program evaluated nearly 7,200 patients treated for up to two years, and showed that lorcaserin consistently produced significant weight loss with excellent tolerability. About two-thirds of patients achieved at least 5% weight loss and over one-third achieved at least 10% weight loss. On average, patients lost 17 to 18 pounds or about 8% of their weight. Secondary endpoints, including body composition, lipids, cardiovascular risk factors and glycemic parameters improved compared to placebo. In addition, heart rate and blood pressure went down. Lorcaserin did not increase the risk of cardiac valvulopathy. Lorcaserin improved quality of life, and there was no signal for depression or suicidal ideation. The only adverse event that exceeded the placebo rate by 5% was generally mild or moderate, transient headache. Based on a normal BMI of 25, patients in the first phase 3 trial lost about one-third of their excess body weight. The average weight loss was 35 pounds or 16% of body weight for the top quartile of patients in the second phase 3 trial.

An immediate-release film-coated 10-mg tablet was developed for the phase 3 clinical trials and commercial launch of lorcaserin, but there remains a need for alternative formulations for oral use. These include rapidly disintegrating or dissolving dosage forms, also known as fast dissolve, fast or rapid melt, and quick disintegrating dosage forms. Rapidly disintegrating or dissolving dosage forms eliminate the need to swallow a tablet and do not require concomitant administration of water. These dosage forms dissolve or disintegrate rapidly in the patient's saliva without chewing. Because of their ease of administration, such compositions are particularly useful for the specific needs of patients who have recently undergone gastric bypass surgery, and patients with a high average daily pill burden. Rapidly disintegrating or dissolving dosage forms are also particularly suited for use with pediatrics, geriatrics, and patients with dysphagia.

In view of the growing demand for compounds useful in the treatment of disorders related to the 5-HT_{2C} receptor, (R)-8-chloro-1-methyl-2,3,4,5-tetrahydro-1H-3-benzazepine has emerged has an important new compound. Accordingly, new formulations of (R)-8-chloro-1-methyl-2,3,4,5-tetrahydro-1H-3-benzazepine are needed. The salts and processes described herein help meet these and other needs.

### SUMMARY OF THE INVENTION

The Biopharmaceutics Classification System (BCS) recommends methods for classifying drugs according to dosage form dissolution, along with the solubility and permeability characteristics of the drug substance. According to the BCS, drug substances are considered highly soluble when the highest dose strength is soluble in <250 mL water over a pH range of 1 to 7.5.

A priori, it is difficult to predict with confidence which salts of a particular drug will be solid, stable, and readily isolable. A fortiori, the solubility characteristics of such salts cannot be predicted with accuracy and must instead must be determined empirically. In the course of preparing the salts of the present invention, many counterions commonly used in the pharmaceutical industry (see e.g. Berge, et al., Journal of Pharmaceutical Sciences, 66:1-19 (1977)) were investigated. Acetate, DL-lactate, ascorbate, D-gluconate, besylate, napsylate, tosylate, isethionate, dichloroacetate, benzoate, esylate, gentisate, hippurate, lactobionate, xinafoate, and sebacate salts of (R)-8-chloro-1-methyl-2,3,4,5-tetrahydro-1H-3-benzazepine were prepared, but all of these failed to crystallize. By contrast, the salts of the present invention are salts of (R)-8-chloro-1-methyl-2,3,4,5-tetrahydro-1H-3-benzazepine which when prepared were discovered to be both crystalline and highly soluble, far exceeding the BCS criterion for characterization as such. Because of their high solubility these salts are useful, inter alia, for preparing rapid-dissolve dosage forms of (R)-8-chloro-1-methyl-2,3,4,5-tetrahydro-1H-3-benzazepine.

One aspect of the present invention pertains to certain salts of (R)-8-chloro-1-methyl-2,3,4,5-tetrahydro-1H-3-benzazepine (Compound 1) and pharmaceutically acceptable solvates and hydrates thereof.

One aspect of the present invention pertains to certain salts of (R)-8-chloro-1-methyl-2,3,4,5-tetrahydro-1H-3-benzazepine (Compound 1).

One aspect of the present invention pertains to salts selected from: (R)-8-chloro-1-methyl-2,3,4,5-tetrahydro-1H-3-benzazepine bisulfate salt; (R)-8-chloro-1-methyl-2,3,4,5-tetrahydro-1H-3-benzazepine hemisulfate salt; (R)-8-chloro-1-methyl-2,3,4,5-tetrahydro-1H-3-benzazepine mesylate salt; (R)-8-chloro-1-methyl-2,3,4,5-tetrahydro-1H-3-benzazepine hydrobromide salt; (R)-8-chloro-1-methyl-2,3,4,5-tetrahydro-1H-3-benzazepine nitrate salt; (R)-8-chloro-1-methyl-2,3,4,5-tetrahydro-1H-3-benzazepine sesqui-oxalate salt-cocrystal; (R)-8-chloro-1-methyl-2,3,4,5-tetrahydro-1H-3-benzazepine adipate salt; (R)-8-chloro-1-methyl-2,3,4,5-tetrahydro-1H-3-benzazepine malonate salt; (R)-8-chloro-1-methyl-2,3,4,5-tetrahydro-1H-3-benzazepine hemimalonate salt; and (R)-8-chloro-1-methyl-2,3,4,5-tetrahydro-1H-3-benzazepine glycolate salt; and pharmaceutically acceptable solvates and hydrates thereof.

One aspect of the present invention pertains to pharmaceutical compositions comprising a salt of the present invention and a pharmaceutically acceptable carrier.

One aspect of the present invention pertains to processes for preparing a pharmaceutical composition comprising admixing a salt of the present invention, and a pharmaceutically acceptable carrier.

One aspect of the present invention pertains to bulk pharmaceutical compositions suitable for the manufacture of dosage forms for weight management, comprising a salt of the present invention, and a pharmaceutically acceptable carrier.

One aspect of the present invention pertains to processes for preparing bulk pharmaceutical compositions suitable for the manufacture of dosage forms for weight management, comprising admixing a salt of the present invention, and a pharmaceutically acceptable carrier.

One aspect of the present invention pertains to dosage forms comprising a therapeutically effective amount of a salt selected from: a pharmaceutically acceptable salt of (R)-8-chloro-1-methyl-2,3,4,5-tetrahydro-1H-3-benzazepine and pharmaceutically acceptable solvates and hydrates thereof, wherein the dosage form is a fast-dissolve dosage form.

One aspect of the present invention pertains to dosage forms comprising a therapeutically effective amount of a salt of the present invention.

One aspect of the present invention pertains to methods for weight management, comprising administering to an individual in need thereof, a therapeutically effective amount of a salt, a pharmaceutical composition, or a dosage form of the present invention.

One aspect of the present invention pertains to the use of salts of the present invention in the manufacture of a medicament for weight management in an individual.

One aspect of the present invention pertains to salts and pharmaceutical compositions of the present invention, for use in a method of treatment of the human or animal body by therapy.

### BRIEF DESCRIPTION OF THE DRAWINGS

FIG. 1: PXRD of Compound 1 Hydrochloride Salt Hemihydrate, Form III.

FIG. 2: DSC of Compound 1 Hydrochloride Salt Hemihydrate, Form III.

FIG. 3: TGA of Compound 1 Hydrochloride Salt Hemihydrate, Form III.

FIG. 4: DMS of Compound 1 Hydrochloride Salt Hemihydrate, Form III.

FIG. 5: PXRD of Compound 1 Bisulfate Salt, Form I.

FIG. 6: DSC and TGA of Compound 1 Bisulfate Salt, Form I.

FIG. 7: DMS of Compound 1 Bisulfate Salt, Form I.

FIG. 8: PXRD of Compound 1 Hemisulfate Salt Hydrate, Form I.

FIG. 9: DSC and TGA of Compound 1 Hemisulfate Salt Hydrate, Form I.

FIG. 10: TGA of Compound 1 Hemisulfate Salt Hydrate, Form I.

FIG. 11: DMS of Compound 1 Hemisulfate Salt Hydrate, Form I.

FIG. 12: PXRD of Compound 1 Mesylate Salt, Form I.

FIG. 13: DSC and TGA of Compound 1 Mesylate Salt, Form I.

FIG. 14: DMS of Compound 1 Mesylate Salt, Form I.

FIG. 15: PXRD of Compound 1 Hydrobromide Salt Hemihydrate, Form I.

FIG. 16: DSC and TGA of Compound 1 Hydrobromide Salt Hemihydrate, Form I.

FIG. 17: DMS of Compound 1 Hydrobromide Salt Hemihydrate, Form I.

FIG. 18: PXRD of Compound 1 Nitrate Salt, Form I.

FIG. 19: DSC and TGA of Compound 1 Nitrate Salt, Form I.

FIG. 20: DMS of Compound 1 Nitrate Salt, Form I.

FIG. 21: PXRD of Compound 1 Sesqui-oxalate Salt-Cocrystal, Form I.

FIG. 22: DSC and TGA of Compound 1 Sesqui-oxalate Salt-Cocrystal, Form I.

FIG. 23: DMS of Compound 1 Sesqui-oxalate Salt-Cocrystal, Form I.

FIG. 24: PXRD of Compound 1 Adipate Salt, Form I.

FIG. 25: DSC and TGA of Compound 1 Adipate Salt, Form I.

FIG. 26: DMS of Compound 1 Adipate Salt, Form I.

FIG. 27: PXRD of Compound 1 Malonate Salt, Form I.

FIG. 28: DSC and TGA of Compound 1 Malonate Salt, Form I.

FIG. 29: DMS of Compound 1 Malonate Salt, Form I.

FIG. 30: PXRD of Compound 1 Hemimalonate Salt, Form I.

FIG. 31: DSC and TGA of Compound 1 Hemimalonate Salt, Form I.

FIG. 32: PXRD of Compound 1 Glycolate Salt, Form I.

FIG. 33: DSC and TGA of Compound 1 Glycolate Salt, Form I.

FIG. 34: DMS of Compound 1 Glycolate Salt, Form I.

### DETAILED DESCRIPTION

It should be appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

### DEFINITIONS

For clarity and consistency, the following definitions will be used throughout this patent document.

The term “agonist” refers to a moiety that interacts with and activates a receptor, such as the 5-HT_{2C} serotonin receptor, and initiates a physiological or pharmacological response characteristic of that receptor.

The term “individual” refers to both humans and non-human mammals. Non-human mammals include but are not limited to rodents such as mice and rats, etc. rabbits, dogs, cats, swine, cattle, sheep, horses, and non-human primates such as monkeys and apes, etc.

The term “pharmaceutical composition” refers to a composition comprising at least one active ingredient; including but not limited to Compound 1 and pharmaceutically acceptable salts, solvates and hydrates thereof, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “therapeutically effective amount” refers to the amount of active compound or pharmaceutical agent that elicits the biological or medicinal response in a tissue, system, animal, individual or human that is being sought by a researcher, veterinarian, medical doctor or other clinician or caregiver or by an individual, which includes one or more of the following:

(1) Preventing the disease, for example, preventing a disease, condition or disorder in an individual that may be predisposed to the disease, condition or disorder but does not yet experience or display the pathology or symptomatology of the disease;

(2) Inhibiting the disease, for example, inhibiting a disease, condition or disorder in an individual that is experiencing or displaying the pathology or symptomatology of the disease, condition or disorder (i.e., arresting further development of the pathology and/or symptomatology); and

(3) Ameliorating the disease, for example, ameliorating a disease, condition or disorder in an individual that is experiencing or displaying the pathology or symptomatology of the disease, condition or disorder (i.e., reversing the pathology and/or symptomatology).

The term “treatment” as used herein refers to one or more of the following:

(1) prevention of a disease, for example, prevention of a disease, condition or disorder in an individual that may be predisposed to the disease, condition or disorder but does not yet experience or display the pathology or symptomatology of the disease;

(2) inhibition of a disease, for example, inhibition of a disease, condition or disorder in an individual that is experiencing or displaying the pathology or symptomatology of the disease, condition or disorder (i.e., arresting further development of the pathology and/or symptomatology); and

(3) amelioration of a disease, for example, amelioration of a disease, condition or disorder in an individual that is experiencing or displaying the pathology or symptomatology of the disease, condition or disorder (i.e., reversing the pathology and/or symptomatology).

Whether an individual is in need of treatment is a judgment made by a caregiver (e.g. nurse practitioner, physician, physician assistant, nurse, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by Compound 1 and pharmaceutically acceptable salts, solvates and hydrates thereof. Accordingly, Compound 1 and pharmaceutically acceptable salts, solvates and hydrates thereof can be used in a protective or preventive manner; or Compound 1 and pharmaceutically acceptable salts, solvates and hydrates thereof can be used to alleviate, inhibit or ameliorate a disease, condition or disorder.

The term “weight management” as used herein refers to controlling body weight and in the context of the present invention is directed toward weight loss and the maintenance of weight loss (also called weight maintenance herein). In addition to controlling body weight, weight management includes controlling parameters related to body weight, for example, BMI, percent body fat and waist circumference. For example, weight management for an individual who is overweight or obese can mean losing weight with the goal of keeping weight in a healthier range. Also, for example, weight management for an individual who is overweight or obese can include losing body fat or circumference around the waist with or without the loss of body weight.

The term “maintenance of weight loss” or “weight maintenance” as used herein refers to preventing, reducing or controlling weight gain after weight loss. It is well known that weight gain often occurs after weight loss. Weight loss can occur, for example, from dieting, exercising, illness, drug treatment, surgery or any combination of these methods, but often an individual that has lost weight will regain some or all of the lost weight. Therefore, weight maintenance in an individual who has lost weight can include preventing weight gain after weight loss, reducing the amount of weigh gained after weight loss, controlling weight gain after weight loss or slowing the rate of weight gain after weight loss.

### Salts of the Invention

The present invention is directed, inter alia, to solid, stable, and readily isolable salts of (R)-8-chloro-1-methyl-2,3,4,5-tetrahydro-1H-3-benzazepine and pharmaceutically acceptable solvates and hydrates thereof. The solid state properties of the crystalline forms of salts the present invention are summarized infra.

One aspect of the present invention pertains to salts selected from: (R)-8-chloro-1-methyl-2,3,4,5-tetrahydro-1H-3-benzazepine bisulfate salt; (R)-8-chloro-1-methyl-2,3,4,5-tetrahydro-1H-3-benzazepine hemisulfate salt; (R)-8-chloro-1-methyl-2,3,4,5-tetrahydro-1H-3-benzazepine mesylate salt; (R)-8-chloro-1-methyl-2,3,4,5-tetrahydro-1H-3-benzazepine hydrobromide salt; (R)-8-chloro-1-methyl-2,3,4,5-tetrahydro-1H-3-benzazepine nitrate salt; (R)-8-chloro-1-methyl-2,3,4,5-tetrahydro-1H-3-benzazepine sesqui-oxalate salt-cocrystal; (R)-8-chloro-1-methyl-2,3,4,5-tetrahydro-1H-3-benzazepine adipate salt; (R)-8-chloro-1-methyl-2,3,4,5-tetrahydro-1H-3-benzazepine malonate salt; (R)-8-chloro-1-methyl-2,3,4,5-tetrahydro-1H-3-benzazepine hemimalonate salt; and (R)-8-chloro-1-methyl-2,3,4,5-tetrahydro-1H-3-benzazepine glycolate salt; and pharmaceutically acceptable solvates and hydrates thereof.

One aspect of the present invention pertains to salts selected from: (R)-8-chloro-1-methyl-2,3,4,5-tetrahydro-1H-3-benzazepine bisulfate salt; (R)-8-chloro-1-methyl-2,3,4,5-tetrahydro-1H-3-benzazepine mesylate salt; (R)-8-chloro-1-methyl-2,3,4,5-tetrahydro-1H-3-benzazepine nitrate salt; (R)-8-chloro-1-methyl-2,3,4,5-tetrahydro-1H-3-benzazepine sesqui-oxalate salt-cocrystal; (R)-8-chloro-1-methyl-2,3,4,5-tetrahydro-1H-3-benzazepine adipate salt; (R)-8-chloro-1-methyl-2,3,4,5-tetrahydro-1H-3-benzazepine malonate salt; (R)-8-chloro-1-methyl-2,3,4,5-tetrahydro-1H-3-benzazepine hemimalonate salt; and (R)-8-chloro-1-methyl-2,3,4,5-tetrahydro-1H-3-benzazepine glycolate salt; and pharmaceutically acceptable solvates and hydrates thereof.

One aspect of the present invention pertains to salts selected from: (R)-8-chloro-1-methyl-2,3,4,5-tetrahydro-1H-3-benzazepine bisulfate salt; (R)-8-chloro-1-methyl-2,3,4,5-tetrahydro-1H-3-benzazepine hemisulfate salt hydrate; (R)-8-chloro-1-methyl-2,3,4,5-tetrahydro-1H-3-benzazepine mesylate salt; (R)-8-chloro-1-methyl-2,3,4,5-tetrahydro-1H-3-benzazepine hydrobromide salt hemihydrate; (R)-8-chloro-1-methyl-2,3,4,5-tetrahydro-1H-3-benzazepine nitrate salt; (R)-8-chloro-1-methyl-2,3,4,5-tetrahydro-1H-3-benzazepine sesqui-oxalate salt-cocrystal; (R)-8-chloro-1-methyl-2,3,4,5-tetrahydro-1H-3-benzazepine adipate salt; (R)-8-chloro-1-methyl-2,3,4,5-tetrahydro-1H-3-benzazepine malonate salt; (R)-8-chloro-1-methyl-2,3,4,5-tetrahydro-1H-3-benzazepine hemimalonate salt; and (R)-8-chloro-1-methyl-2,3,4,5-tetrahydro-1H-3-benzazepine glycolate salt.

One aspect of the present invention pertains to salts selected from: (R)-8-chloro-1-methyl-2,3,4,5-tetrahydro-1H-3-benzazepine hemisulfate salt hydrate; and (R)-8-chloro-1-methyl-2,3,4,5-tetrahydro-1H-3-benzazepine hydrobromide salt hemihydrate.

One aspect of the present invention pertains to (R)-8-chloro-1-methyl-2,3,4,5-tetrahydro-1H-3-benzazepine bisulfate salt.

One aspect of the present invention pertains to (R)-8-chloro-1-methyl-2,3,4,5-tetrahydro-1H-3-benzazepine hemisulfate salt hydrate.

One aspect of the present invention pertains to (R)-8-chloro-1-methyl-2,3,4,5-tetrahydro-1H-3-benzazepine mesylate salt.

One aspect of the present invention pertains to (R)-8-chloro-1-methyl-2,3,4,5-tetrahydro-1H-3-benzazepine hydrobromide salt hemihydrate. One aspect of the present invention pertains to (R)-8-chloro-1-methyl-2,3,4,5-tetrahydro-1H-3-benzazepine nitrate salt.

One aspect of the present invention pertains to (R)-8-chloro-1-methyl-2,3,4,5-tetrahydro-1H-3-benzazepine sesqui-oxalate salt-cocrystal.

One aspect of the present invention pertains to (R)-8-chloro-1-methyl-2,3,4,5-tetrahydro-1H-3-benzazepine adipate salt.

One aspect of the present invention pertains to (R)-8-chloro-1-methyl-2,3,4,5-tetrahydro-1H-3-benzazepine malonate salt.

One aspect of the present invention pertains to (R)-8-chloro-1-methyl-2,3,4,5-tetrahydro-1H-3-benzazepine hemimalonate salt. One aspect of the present invention pertains to (R)-8-chloro-1-methyl-2,3,4,5-tetrahydro-1H-3-benzazepine glycolate salt.

One aspect of the present invention pertains to pharmaceutical compositions comprising a salt of the present invention.

One aspect of the present invention pertains to process for preparing a pharmaceutical composition comprising admixing a salt of the present invention and a pharmaceutically acceptable carrier.

One aspect of the present invention pertains to methods for weight management, comprising administering to an individual in need thereof, a therapeutically effective amount of a salt of the present invention.

One aspect of the present invention pertains to the use of salts of the present invention, in the manufacture of a medicament for weight management in an individual.

One aspect of the present invention pertains to salts of the present invention, for use in a method of treatment of the human or animal body by therapy.

One aspect of the present invention pertains to salts of the present invention, for use in a method of weight management.

One aspect of the present invention pertains to salts of the present invention, for use in a method of weight loss.

One aspect of the present invention pertains to salts of the present invention, for use in a method of maintenance of weight loss.

One aspect of the present invention pertains to salts of the present invention, for use in a method of decreasing food consumption.

One aspect of the present invention pertains to salts of the present invention, for use in a method of increasing meal-related satiety.

One aspect of the present invention pertains to salts of the present invention, for use in a method of reducing pre-meal hunger.

One aspect of the present invention pertains to salts of the present invention, for use in a method of reducing intra-meal food intake.

One aspect of the present invention pertains to salts of the present invention, for use in a method of weight management further comprising a reduced-calorie diet.

One aspect of the present invention pertains to salts of the present invention, for use in a method of weight management further comprising a program of regular exercise.

One aspect of the present invention pertains to salts of the present invention, for use in a method of weight management further comprising a reduced-calorie diet and a program of regular exercise.

One aspect of the present invention pertains to salts of the present invention, for use in a method of weight management in an obese patient with an initial body mass index ≧30 kg/m².

One aspect of the present invention pertains to salts of the present invention, for use in a method of weight management in an overweight patient with an initial body mass index ≧27 kg/m² in the presence of at least one weight related co-morbid condition.

One aspect of the present invention pertains to salts of the present invention, for use in a method of weight management in an overweight patient with an initial body mass index ≧27 kg/m² in the presence of at least one weight related co-morbid condition selected from: hypertension, dyslipidemia, cardiovascular disease, glucose intolerance, and sleep apnea.

One aspect of the present invention pertains to salts of the present invention, for use in a method of weight management in an individual with an initial body mass index ≧30 kg/m².

One aspect of the present invention pertains to salts of the present invention, for use in a method of weight management in an individual with an initial body mass index ≧27 kg/m².

One aspect of the present invention pertains to salts of the present invention, for use in a method of weight management in an individual with an initial body mass index ≧27 kg/m² in the presence of at least one weight related co-morbid condition.

One aspect of the present invention pertains to salts of the present invention, for use in a method of weight management in an individual with an initial body mass index ≧27 kg/m² in the presence of at least one weight related co-morbid condition selected from: hypertension, dyslipidemia, cardiovascular disease, glucose intolerance, and sleep apnea.

One aspect of the present invention pertains to salts of the present invention, for use in a method of weight management in an individual with an initial body mass index ≧25 kg/m².

One aspect of the present invention pertains to salts of the present invention, for use in a method of weight management in an individual with an initial body mass index ≧25 kg/m² in the presence of at least one weight related co-morbid condition.

One aspect of the present invention pertains to salts of the present invention, for use in a method of weight management in an individual with an initial body mass index ≧25 kg/m² in the presence of at least one weight related co-morbid condition selected from: hypertension, dyslipidemia, cardiovascular disease, glucose intolerance, and sleep apnea.

One aspect of the present invention pertains to salts of the present invention, for use in a method of weight management in combination with phentermine.

One aspect of the present invention pertains to salts and pharmaceutical compositions of the present invention for use in a method of treatment of the human or animal body by therapy.

In some embodiments, the salts and pharmaceutical compositions are for use in a method of weight management.

In some embodiments, the salts and pharmaceutical compositions are for use in a method of weight loss.

In some embodiments, the salts and pharmaceutical compositions are for use in a method of maintenance of weight loss.

In some embodiments, the salts and pharmaceutical compositions are for use in a method of decreasing food consumption.

In some embodiments, the salts and pharmaceutical compositions are for use in a method of increasing meal-related satiety.

In some embodiments, the salts and pharmaceutical compositions are for use in a method of reducing pre-meal hunger.

In some embodiments, the salts and pharmaceutical compositions are for use in a method of educing intra-meal food intake.

In some embodiments, the salts and pharmaceutical compositions are for use in a method of weight management further comprising a reduced-calorie diet.

In some embodiments, the salts and pharmaceutical compositions are for use in a method of weight management further comprising a program of regular exercise.

In some embodiments, the salts and pharmaceutical compositions are for use in a method of weight management further comprising a reduced-calorie diet and a program of regular exercise.

In some embodiments, the salts and pharmaceutical compositions are for use in a method of weight management in an obese patient with an initial body mass index ≧30 kg/m².

In some embodiments, the salts and pharmaceutical compositions are for use in a method of weight management in an overweight patient with an initial body mass index ≧27 kg/m² in the presence of at least one weight related co-morbid condition.

In some embodiments, the salts and pharmaceutical compositions are for use in a method of weight management in an overweight patient with an initial body mass index ≧27 kg/m² in the presence of at least one weight related co-morbid condition selected from: hypertension, dyslipidemia, cardiovascular disease, glucose intolerance, and sleep apnea.

In some embodiments, the salts and pharmaceutical compositions are for use in a method of weight management in an individual with an initial body mass index ≧30 kg/m².

In some embodiments, the salts and pharmaceutical compositions are for use in a method of weight management in an individual with an initial body mass index ≧27 kg/m².

In some embodiments, the salts and pharmaceutical compositions are for use in a method of weight management in an individual with an initial body mass index ≧27 kg/m² in the presence of at least one weight related co-morbid condition.

In some embodiments, the salts and pharmaceutical compositions are for use in a method of weight management in an individual with an initial body mass index ≧27 kg/m² in the presence of at least one weight related co-morbid condition selected from: hypertension, dyslipidemia, cardiovascular disease, glucose intolerance, and sleep apnea.

In some embodiments, the salts and pharmaceutical compositions are for use in a method of weight management in an individual with an initial body mass index ≧25 kg/m².

In some embodiments, the salts and pharmaceutical compositions are for use in a method of weight management in an individual with an initial body mass index ≧25 kg/m² in the presence of at least one weight related co-morbid condition.

In some embodiments, the salts and pharmaceutical compositions are for use in a method of weight management in an individual with an initial body mass index ≧25 kg/m² in the presence of at least one weight related co-morbid condition selected from: hypertension, dyslipidemia, cardiovascular disease, glucose intolerance, and sleep apnea.

In some embodiments, the salts and pharmaceutical compositions are for use in a method of weight management in combination with phentermine.

### Crystalline Salts

Polymorphism is the ability of a substance to exist as two or more crystalline phases that have different arrangements and/or conformations of the molecules in the crystal lattice. Polymorphs show the same properties in the liquid or gaseous state but they may behave differently in the solid state.

Besides single-component polymorphs, drugs can also exist as salts and other multicomponent crystalline phases. For example, solvates and hydrates may contain an API host and either solvent or water molecules, respectively, as guests. Analogously, when the guest compound is a solid at room temperature, the resulting form is often called a cocrystal. Salts, solvates, hydrates, and cocrystals may show polymorphism as well. Crystalline phases that share the same API host, but differ with respect to their guests, may be referred to as pseudopolymorphs of one another.

Solvates contain molecules of the solvent of crystallization in a definite crystal lattice. Solvates, in which the solvent of crystallization is water, are termed hydrates. Because water is a constituent of the atmosphere, hydrates of drugs may be formed rather easily.

Recently, polymorph screens of 245 compounds revealed that about 90% of them exhibited multiple solid forms. Overall, approximately half the compounds were polymorphic, often having one to three forms. About one-third of the compounds formed hydrates, and about one-third formed solvates. Data from cocrystal screens of 64 compounds showed that 60% formed cocrystals other than hydrates or solvates. (G. P. Stahly, Crystal Growth & Design (2007), 7(6), 1007-1026.)

The present invention is directed, inter alia, to crystalline salts of (R)-8-chloro-1-methyl-2,3,4,5-tetrahydro-1H-3-benzazepine and hydrates and solvates thereof. The crystalline forms of the salts of the present invention can be identified by unique solid state signatures with respect to, for example, differential scanning calorimetry (DSC), X-ray powder diffraction (PXRD), and other solid state methods. Further characterization with respect to water or solvent content of the crystalline salts of the present invention can be gauged by any of the following methods for example, thermogravimetric analysis (TGA), DSC and the like. For DSC, it is known that the temperatures observed will depend upon sample purity, the rate of temperature change, as well as sample preparation technique and the particular instrument employed. Thus, the values reported herein relating to DSC thermograms can vary by about ±6° C. The values reported herein relating to DSC thermograms can also vary by about ±20 joules per gram. For PXRD, the relative intensities of the peaks can vary, depending upon the sample preparation technique, the sample mounting procedure and the particular instrument employed. Moreover, instrument variation and other factors can often affect the 2θ values. Therefore, the peak assignments of diffraction patterns can vary by about ±0.2 °2θ. The relative intensities of the reported peaks can also vary. For TGA, the features reported herein can vary by about ±5° C. The TGA features reported herein can also vary by about ±2% weight change due to, for example, sample variation. Further characterization with respect to hygroscopicity of the crystalline salt can be gauged by, for example, dynamic moisture sorption (DMS). The DMS features reported herein can vary by about ±5% relative humidity. The DMS features reported herein can also vary by about ±5% weight change. The deliquescence relative humidity (DRH) measurements by water activity meter are sensitive to sample quality and quantity. The DRH measurements reported herein can vary by about ±5% RH.

### Compound 1 Hydrochloride Salt Hemihydrate, Form III.

The physical properties of Form III of Compound 1 hydrochloride salt hemihydrate are summarized in Table 1 below.

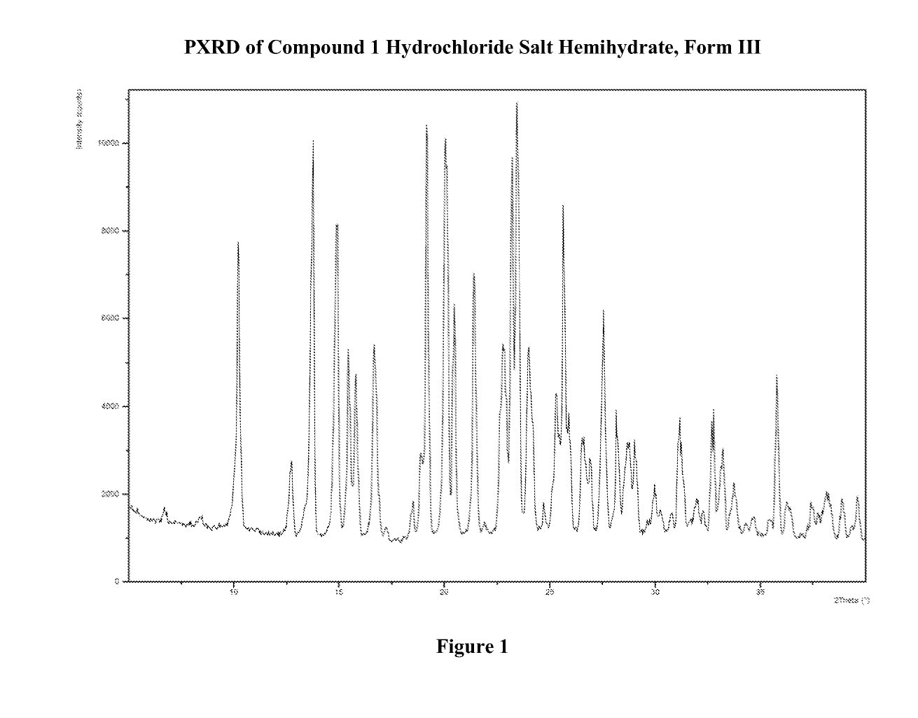

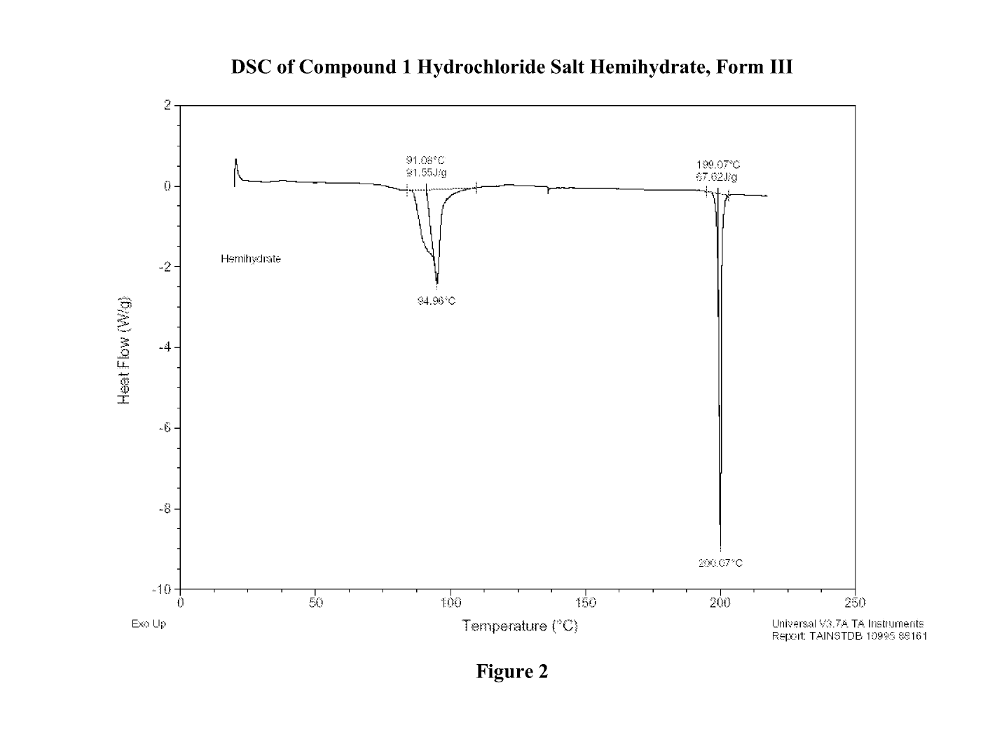

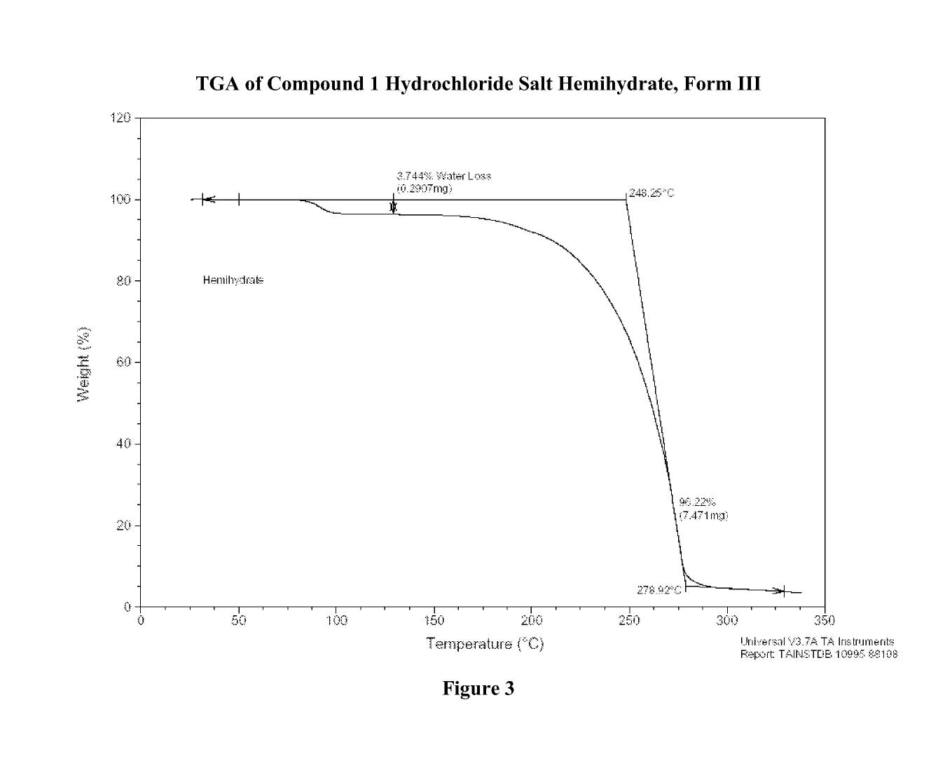

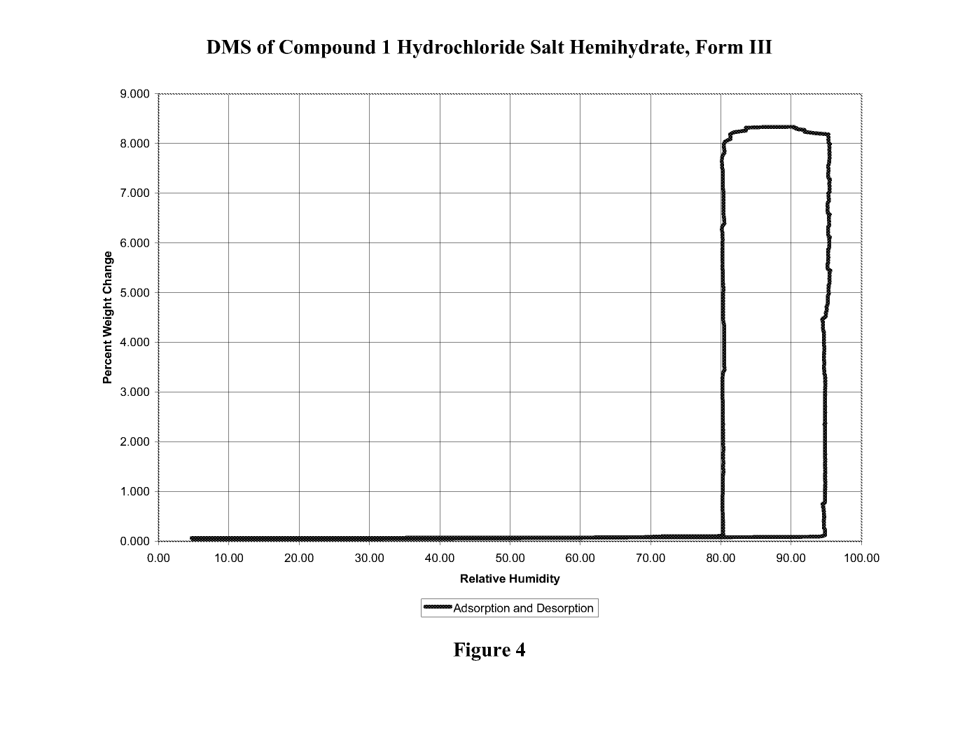

TABLE 1

| | Compound 1 Hydrochloride Salt Hemihydrate, Form III |
|---|---|
| PXRD | FIG. 1: Peaks at 13.7°, 14.9°, 15.4°, 15.8°, 16.7°, 18.9° 2θ |
| DSC | FIG. 2: 95° C. (dehydration); 200° C. (melt) |
| TGA | FIG. 3: 3.7% water loss |
| DMS | FIG. 4: non-hygroscopic |

Compound 1 hydrochloride salt hemihydrate, Form III displays a dehydration feature calculated as a 3.7% weight loss which is consistent with the theoretical weight loss of 3.7% for a hemihydrate. Analysis by DSC further confirms the TGA results, where Compound 1 hydrochloride salt hemihydrate, Form III shows a dehydration event at about 95° C. and a melting/decomposition endotherm at about 200-201° C.

DVS data shows that Compound 1 hydrochloride salt hemihydrate, Form III is substantially non-hygroscopic, adsorbing less than 0.5 wt % water out to and including the 90% RH hold at 25° C. and the XRPD pattern showed no change in crystalline form of the salt after the DVS cycle.

Certain X-ray powder diffraction peaks for Compound 1 hydrochloride salt hemihydrate, Form III are shown in Table 2 below.

TABLE 2
Pos. (° 2θ)

10.2
12.7
13.7
14.9
15.4
15.8
16.7
18.5
18.9
19.2
20.1
25.3
25.7
26.0
26.5
26.9
27.6
28.2
20.5
21.4
22.8
23.2
23.5
24.0
24.2
24.7
29.0
30.0
30.3
30.8
31.1
32.0
32.3
32.7
33.3
33.8
35.8

Form III of Compound 1 hydrochloride salt hemihydrate can be prepared as described in Example 11.

### Compound 1 Bisulfate Salt

One aspect of the present invention pertains to (R)-8-chloro-1-methyl-2,3,4,5-tetrahydro-1H-3-benzazepine bisulfate salt, Form I (Compound 1 bisulfate salt, Form I). The physical properties of Compound 1 bisulfate salt, Form I of are summarized in Table 3 below.

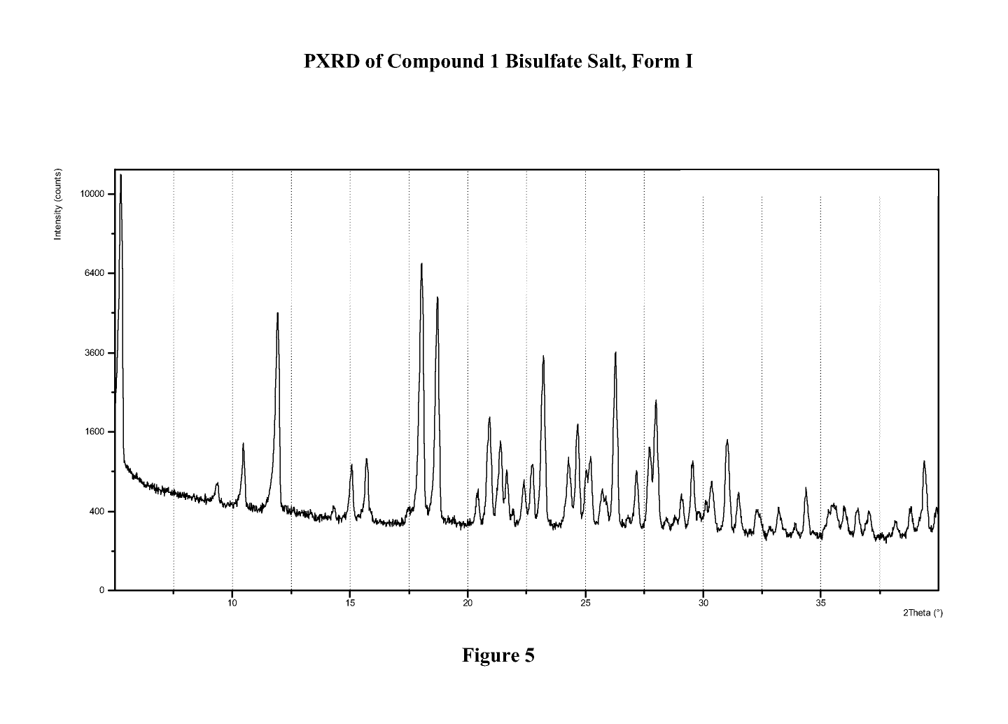

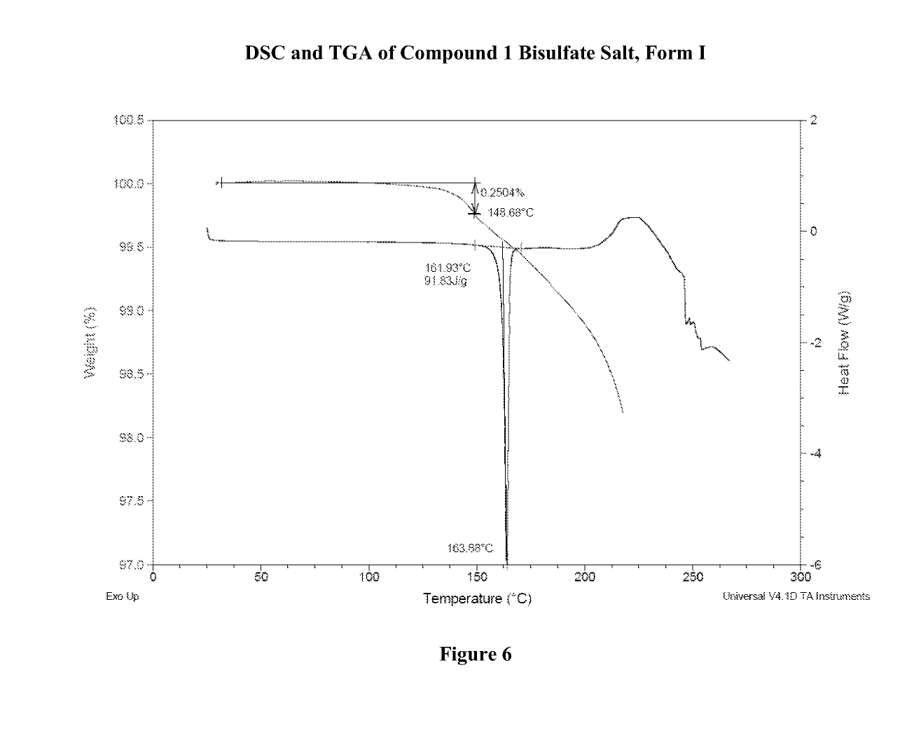

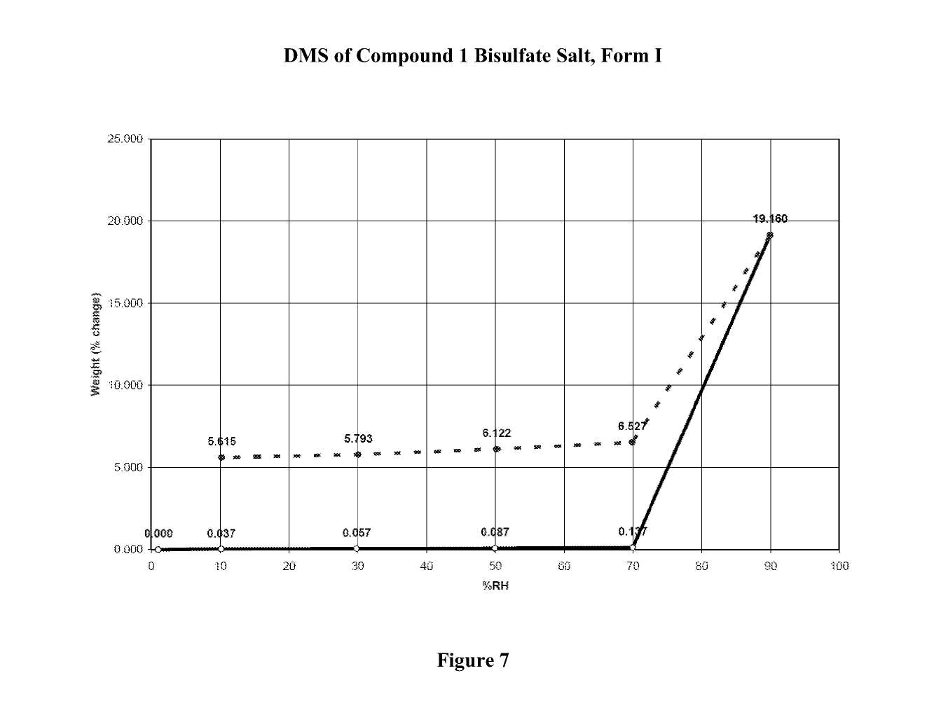

TABLE 3

| | Compound 1 Bisulfate Salt, Form I |
|---|---|
| PXRD | FIG. 5: Peaks of 10% relative intensity at 5.27, |
| | 11.93, 18.05, 18.71, 20.92, 21.39, 23.21, 24.66, 26.28, |
| | 27.73, 28.00, and 31.02° 2θ |
| TGA | FIG. 6: <0.25% weight loss up to about 149° C. |
| DSC | FIG. 6: extrapolated onset temperature: 162° C.; |
| | enthalpy of fusion 92 J/g |
| DMS | FIG. 7: deliquescent between 70 and 90% RH |

DSC of Form I of Compound 1 bisulfate salt showed a melting onset temperature of 162° C. and an enthalpy of fusion 92 J/g. By TGA the sample lost a small amount of weight just prior to melting and continued to lose weight during and after the melt.

Dynamic Moisture-Sorption (DMS) analysis and deliquescence evaluation of Form I of Compound 1 bisulfate salt showed no significant amount of water was absorbed at 70% RH or lower relative humidity. However, the sample absorbed significant water at the 90% RH hold, indicating deliquescence is likely occurring at relative humidity between 70 and 90% RH. The hysteresis shown in FIG. 7 represents outer crust formation during desorption, which leads to limited diffusion of water from the sample during the desorption cycle. This phenomenon is not uncommon for deliquescing compounds.

Certain X-ray powder diffraction peaks for Form I of Compound 1 bisulfate salt are shown in Table 4 below.

TABLE 4

| Pos. (° 2θ) | Rel. Int. (%) |
|---|---|
| 5.27 | 100.00 |
| 9.37 | 2.69 |
| 10.48 | 8.80 |
| 11.93 | 44.48 |
| 14.31 | 1.44 |
| 15.08 | 6.91 |
| 15.71 | 8.05 |
| 17.47 | 1.58 |
| 18.05 | 63.18 |
| 18.71 | 50.45 |
| 20.42 | 3.39 |
| 20.92 | 15.96 |
| 21.39 | 11.23 |
| 21.65 | 6.63 |
| 21.93 | 1.41 |
| 22.39 | 5.12 |
| 22.74 | 7.73 |
| 23.21 | 31.29 |
| 24.29 | 8.41 |
| 24.66 | 15.05 |
| 25.04 | 6.81 |
| 25.21 | 8.82 |
| 25.72 | 4.41 |
| 26.28 | 32.91 |
| 26.81 | 1.58 |
| 27.17 | 6.91 |
| 27.73 | 10.84 |
| 28.00 | 20.31 |
| 28.44 | 1.35 |
| 28.83 | 1.49 |
| 29.08 | 3.74 |
| 29.55 | 8.62 |
| 30.12 | 3.14 |
| 30.35 | 5.63 |
| 31.02 | 12.18 |
| 31.51 | 4.12 |
| 32.22 | 1.97 |
| 32.84 | 0.82 |
| 33.21 | 2.68 |
| 33.91 | 1.19 |
| 34.36 | 4.80 |
| 35.52 | 2.88 |
| 35.98 | 2.72 |
| 36.59 | 2.60 |
| 37.04 | 2.27 |
| 38.17 | 1.45 |
| 38.76 | 2.44 |
| 39.39 | 8.83 |

One aspect of the present invention is directed to a Compound 1 bisulfate salt having an X-ray powder diffraction pattern comprising a peak, in terms of 2θ, at about 5.27°. In some embodiments, the salt has an X-ray powder diffraction pattern comprising a peak, in terms of 2θ, at about 18.05°. In some embodiments, the salt has an X-ray powder diffraction pattern comprising peaks, in terms of 2θ, at about 5.27° and about 18.05°. In some embodiments, the salt has an X-ray powder diffraction pattern comprising peaks, in terms of 2θ, at about 5.27° and about 18.71°. In some embodiments, the salt has an X-ray powder diffraction pattern comprising peaks, in terms of 2θ, at about 5.27°, about 18.05°, and about 18.71°. In some embodiments, the salt has an X-ray powder diffraction pattern comprising peaks, in terms of 2θ, at about 5.27°, about 18.05°, about 18.71°, about 11.93°, about 26.28°, about 23.21°, and about 28.00°. In some embodiments, the salt has an X-ray powder diffraction pattern comprising peaks, in terms of 2θ, at about 5.27°, about 18.05°, about 18.71°, about 11.93°, about 26.28°, about 23.21°, about 28.00°, about 20.92°, about 24.66°, and about 31.02°. One aspect of the present invention is directed to a Compound 1 bisulfate salt having an X-ray powder diffraction pattern comprising one or more peaks listed in Table 4. In some embodiments, the salt has an X-ray powder diffraction pattern substantially as shown in FIG. 5, wherein by “substantially” is meant that the reported peaks can vary by about ±0.2 °2θ and also that the relative intensities of the reported peaks can vary.

In some embodiments, the Compound 1 bisulfate salt has a differential scanning calorimetry thermogram comprising an endotherm with an extrapolated onset temperature between about 145° C. and about 175° C. In some embodiments, the Compound 1 bisulfate salt has a differential scanning calorimetry thermogram comprising an endotherm with an extrapolated onset temperature at about 162° C. In some embodiments, the Compound 1 bisulfate salt has a differential scanning calorimetry thermogram comprising an endotherm with an associated heat flow of about 92 joules per gram. In some embodiments, the Compound 1 bisulfate salt has a thermogravimetric analysis profile substantially as shown in FIG. 6, wherein by “substantially” is meant that the reported TGA features can vary by about ±5° C. and by about ±2% weight change.

In some embodiments, the Compound 1 bisulfate salt has a differential scanning calorimetry thermogram substantially as shown in FIG. 6, wherein by “substantially” is meant that the reported DSC features can vary by about ±6° C. and by about ±20 joules per gram.

In some embodiments, the Compound 1 bisulfate salt has a dynamic moisture sorption profile substantially as shown in FIG. 7, wherein by “substantially” is meant that the reported DMS features can vary by about ±5% relative humidity and by about ±5% weight change.

Form I of Compound 1 bisulfate salt can be prepared by any of the suitable procedures known in the art for preparing crystalline polymorphs. In some embodiments Form I of Compound 1 bisulfate salt can be prepared as described in Example 1. In some embodiments, Form I of Compound 1 bisulfate salt can be prepared by heating Compound 1 bisulfate salt containing one or more crystalline forms other than Form I. In some embodiments, Form I of Compound 1 bisulfate salt can be prepared by recrystallizing crystalline Compound 1 bisulfate salt containing one or more crystalline forms other than Form I.

### Compound 1 Hemisulfate Salt Hydrate

One aspect of the present invention pertains to (R)-8-chloro-1-methyl-2,3,4,5-tetrahydro-1H-3-benzazepine hemisulfate salt hydrate, Form I (Compound 1 hemisulfate salt hydrate, Form I). The physical properties of Compound 1 hemisulfate salt hydrate, Form I of are summarized in Table 5 below.

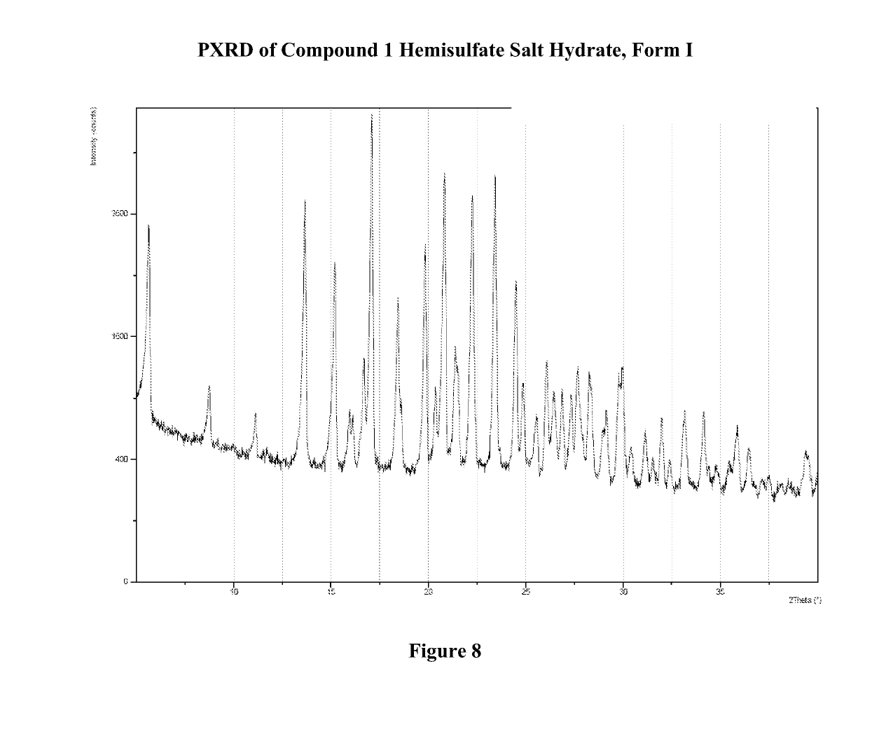

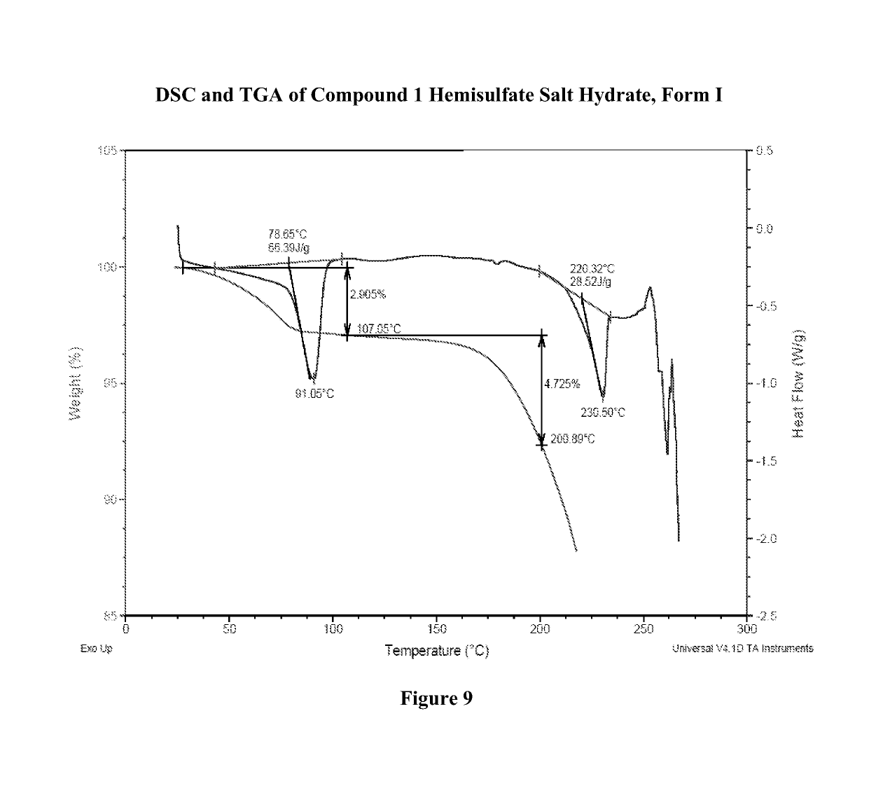

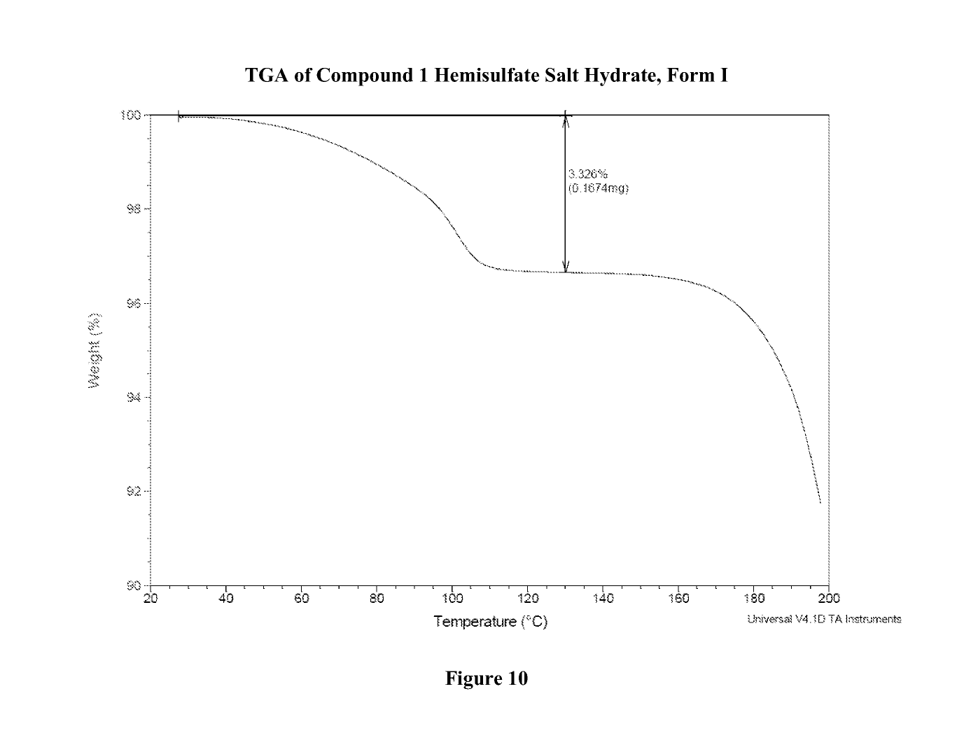

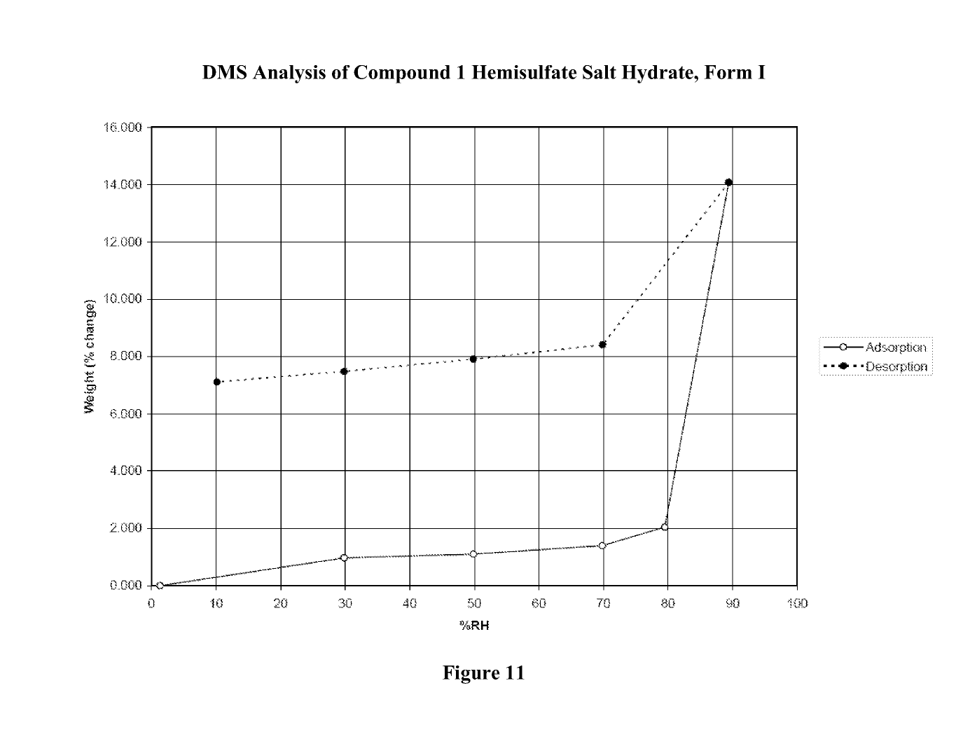

TABLE 5

| | Compound 1 Hemisulfate Salt Hydrate, Form I |
|---|---|
| PXRD | FIG. 8: Peaks of >20% relative intensity at 5.64, |
| | 13.66, 15.20, 17.10, 18.44, 19.84, 20.83, 21.39, 22.26, |
| | 23.43, and 24.50° 2θ |
| DSC | FIG. 9: Broad endotherm starting near 50° C. and an |
| | extrapolated onset temperature at about 79° C. |
| TGA | FIGS. 9 and 10: Between 2.9 and 3.3% weight loss up |
| | to about 130° C. |
| DMS | FIG. 11: deliquescent between 80 and 90% RH |

Form I of Compound 1 hemisulfate salt hydrate, was a hydrated crystalline material with a dehydration onset temperature below 50° C. The weight loss by TGA ranged from 2.9% to 3.3% for two independent samples, the latter being close to a hemihydrate with respect to Compound 1.

Form I of Compound 1 hemisulfate salt hydrate was slightly hygroscopic by DMS up to 80% RH, (˜2% water up to and including the 80% RH hold). DMS also showed the compound picked up significantly more water at the 90% RH hold, indicating the compound was deliquescent between 80 and 90% RH. This was consistent with the measured DRH value 83% RH at 25° C., determined by water activity measurement of a sample saturated in water with excess solid.

Certain X-ray powder diffraction peaks for Form I of Compound 1 hemisulfate salt hydrate are shown in Table 6 below.

TABLE 6

| Pos. (° 2θ) | Rel. Int. (%) |
|---|---|
| 5.64 | 48.94 |
| 8.74 | 10.40 |
| 11.12 | 6.26 |
| 13.66 | 64.72 |
| 15.20 | 43.37 |
| 15.95 | 7.72 |
| 16.12 | 6.87 |
| 16.69 | 18.42 |
| 17.10 | 100.00 |
| 18.44 | 33.16 |
| 18.62 | 9.24 |
| 19.84 | 49.86 |
| 20.37 | 12.48 |
| 20.83 | 74.88 |
| 21.39 | 21.48 |
| 21.50 | 16.69 |
| 22.26 | 66.24 |
| 23.43 | 74.85 |
| 24.50 | 38.93 |
| 24.86 | 13.98 |
| 25.56 | 8.14 |
| 26.08 | 18.77 |
| 26.45 | 12.36 |
| 26.88 | 13.14 |
| 27.34 | 12.25 |
| 27.67 | 17.60 |
| 28.28 | 15.95 |
| 28.39 | 12.14 |
| 28.93 | 6.26 |
| 29.15 | 9.70 |
| 29.78 | 16.65 |
| 29.96 | 17.78 |
| 30.42 | 4.24 |
| 31.14 | 6.85 |
| 31.54 | 3.27 |
| 31.99 | 9.19 |
| 32.40 | 3.51 |
| 33.17 | 10.64 |
| 34.14 | 10.24 |
| 34.78 | 2.80 |
| 35.46 | 3.21 |
| 35.87 | 8.35 |
| 36.47 | 4.95 |
| 37.12 | 1.53 |
| 37.50 | 2.01 |
| 38.13 | 1.25 |
| 38.56 | 1.10 |
| 39.37 | 4.42 |
| 39.55 | 3.48 |

One aspect of the present invention is directed to a Compound 1 hemisulfate salt hydrate having an X-ray powder diffraction pattern comprising a peak, in terms of 2θ, at about 17.10°. In some embodiments, the salt has an X-ray powder diffraction pattern comprising a peak, in terms of 2θ, at about 20.83°. In some embodiments, the salt has an X-ray powder diffraction pattern comprising peaks, in terms of 2θ, at about 17.10° and about 20.83°. In some embodiments, the salt has an X-ray powder diffraction pattern comprising peaks, in terms of 2θ, at about 17.10° and about 23.43°. In some embodiments, the salt has an X-ray powder diffraction pattern comprising peaks, in terms of 2θ, at about 17.10°, about 20.83°, and about 23.43°. In some embodiments, the salt has an X-ray powder diffraction pattern comprising peaks, in terms of 2θ, at about 17.10°, about 20.83°, about 23.43°, about 22.26°, about 13.66°, about 19.84°, and about 5.64°. In some embodiments, the salt has an X-ray powder diffraction pattern comprising peaks, in terms of 2θ, at about 17.10°, about 20.83°, about 23.43°, about 22.26°, about 13.66°, about 19.84°, about 5.64°, about 15.20°, about 24.50°, and about 18.44°. One aspect of the present invention is directed to a Compound 1 hemisulfate salt hydrate having an X-ray powder diffraction pattern comprising one or more peaks listed in Table 6. In some embodiments, the salt has an X-ray powder diffraction pattern substantially as shown in FIG. 8, wherein by “substantially” is meant that the reported peaks can vary by about ±0.2 °2θ and also that the relative intensities of the reported peaks can vary.

In some embodiments, the Compound 1 hemisulfate salt hydrate has a differential scanning calorimetry thermogram comprising an endotherm with an extrapolated onset temperature between about 60° C. and about 90° C. In some embodiments, the Compound 1 hemisulfate salt hydrate has a differential scanning calorimetry thermogram comprising an endotherm with an extrapolated onset temperature at about 79° C. In some embodiments, the Compound 1 hemisulfate salt hydrate has a differential scanning calorimetry thermogram comprising an endotherm with an associated heat flow of about 66 joules per gram. In some embodiments, the Compound 1 hemisulfate salt hydrate has a differential scanning calorimetry thermogram substantially as shown in FIG. 9, wherein by “substantially” is meant that the reported DSC features can vary by about ±6° C. and by about ±20 joules per gram.

In some embodiments, the Compound 1 hemisulfate salt hydrate has a thermogravimetric analysis profile substantially as shown in FIG. 9, wherein by “substantially” is meant that the reported TGA features can vary by about ±5° C. and by about ±2% weight change.

In some embodiments, the Compound 1 hemisulfate salt hydrate has a thermogravimetric analysis profile substantially as shown in FIG. 10, wherein by “substantially” is meant that the reported TGA features can vary by about ±5° C. and by about ±2% weight change.

In some embodiments, the Compound 1 hemisulfate salt hydrate has a dynamic moisture sorption profile substantially as shown in FIG. 11, wherein by “substantially” is meant that the reported DMS features can vary by about ±5% relative humidity and by about ±5% weight change.

Form I of Compound 1 hemisulfate salt hydrate can be prepared by any of the suitable procedures known in the art for preparing crystalline polymorphs. In some embodiments Form I of Compound 1 hemisulfate salt hydrate can be prepared as described in Example 2. In some embodiments, Form I of Compound 1 hemisulfate salt hydrate can be prepared by slurrying crystalline Compound 1 hemisulfate salt containing one or more crystalline forms other than Form I. In some embodiments, Form I of Compound 1 hemisulfate salt hydrate can be prepared by recrystallizing crystalline Compound 1 hemisulfate salt containing one or more crystalline forms other than Form I.

### Compound 1 Mesylate Salt

One aspect of the present invention pertains to (R)-8-chloro-1-methyl-2,3,4,5-tetrahydro-1H-3-benzazepine mesylate salt is Form I (Compound 1 mesylate salt, Form I). The physical properties of Compound 1 mesylate salt, Form I of are summarized in Table 7 below.

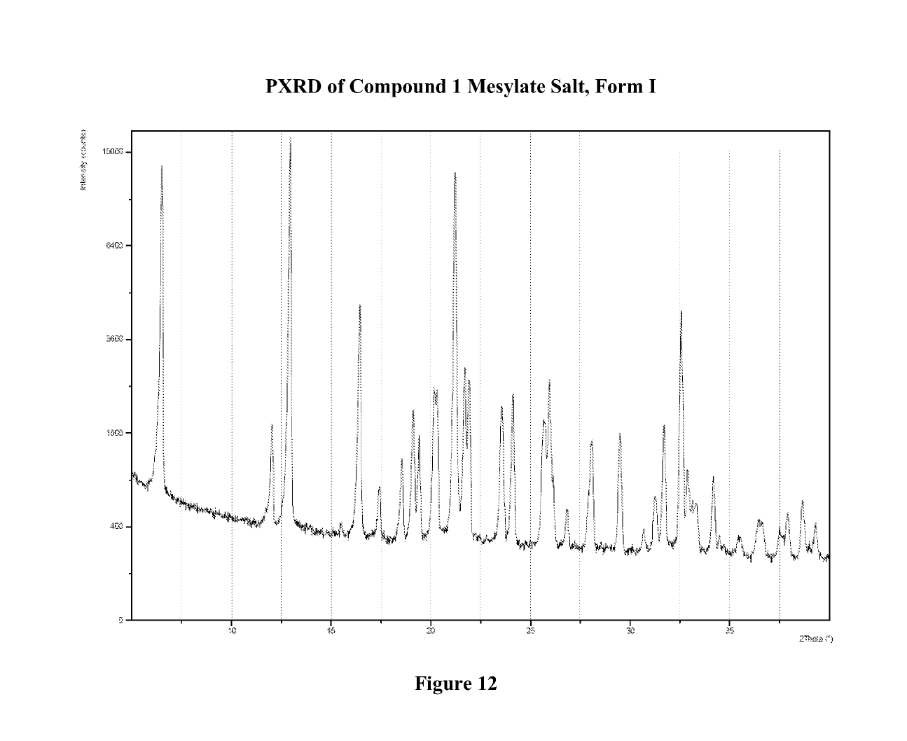

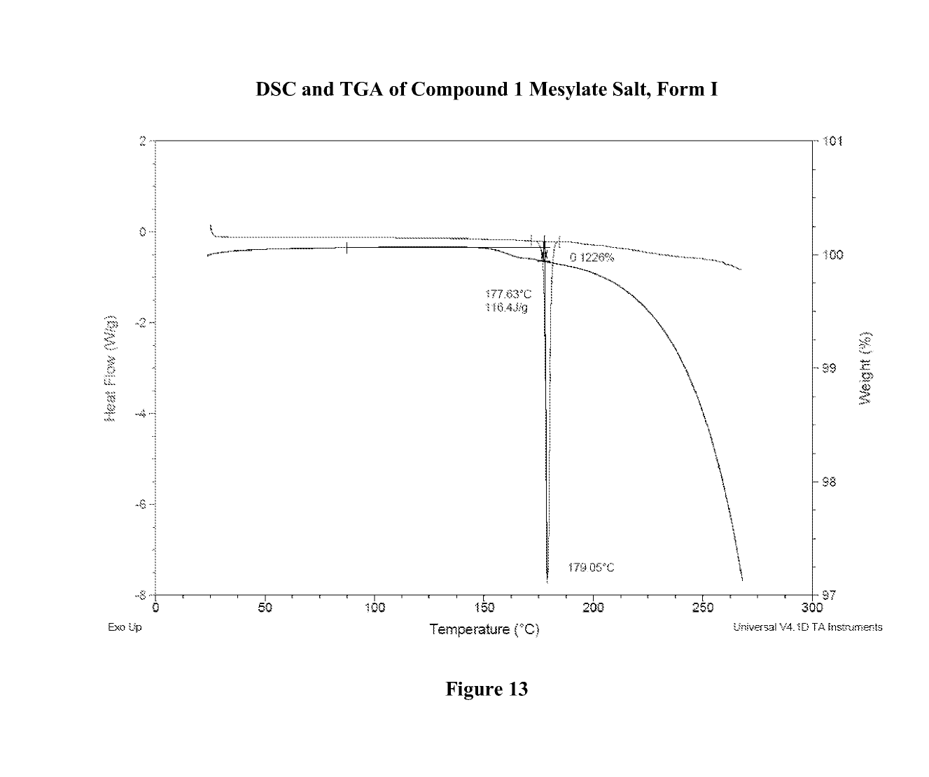

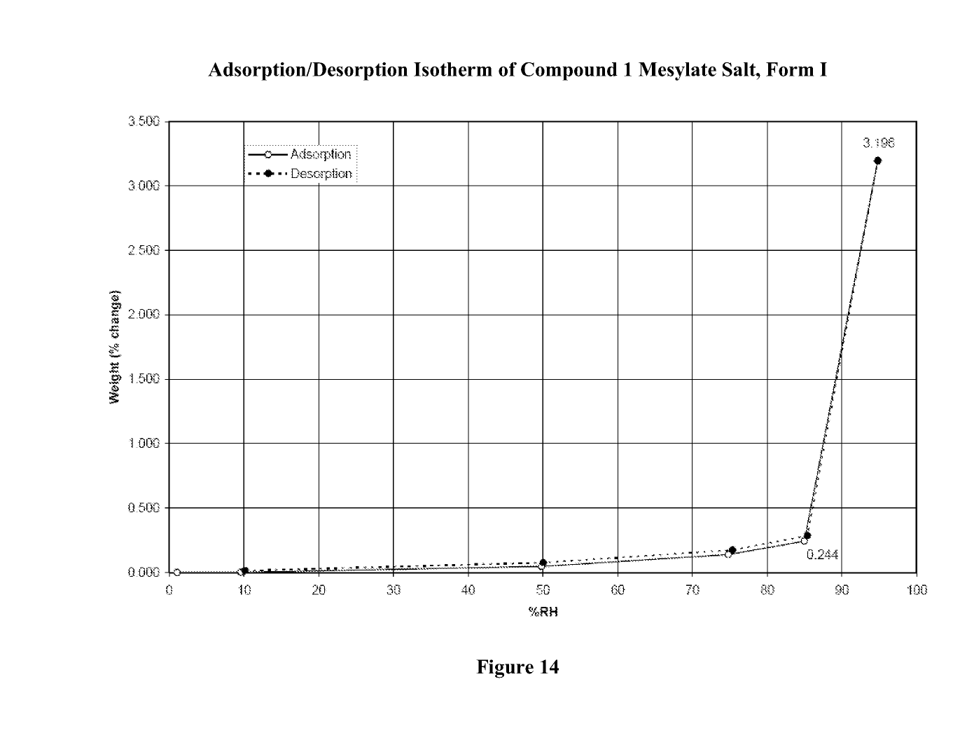

TABLE 7

| | Compound 1 Mesylate Salt, Form I |
|---|---|
| PXRD | FIG. 12: Peaks of ≧20% relative intensity at 6.51, 12.95, |
| | 16.44, 20.19, 20.31, 21.22, 21.71, 21.93, 24.13, 25.96, and |
| | 32.57° 2θ |
| TGA | FIG. 13: 0.12% weight-loss just prior to the melting onset |
| DSC | FIG. 13: extrapolated onset temperature about 178° C.; |
| | enthalpy of fusion 116.4 J/g |
| DMS | FIG. 14: non-hygroscopic up to 85% RH; slightly |
| | hygroscopic up to 95% RH |

Compound 1 mesylate salt, Form I had a melting onset about 178° C. It appeared to hold a small amount of residual solvent by TGA, losing about 0.12% weight just prior to the melting onset.

Compound 1 mesylate salt, Form I was non-hygroscopic up to 85% RH at 25° C., picking up less than 0.25% in weight. However, at 95% RH it picked up about 3.2% weight. This is consistent with the DRH, 93.8% RH at 25° C., determined by water activity measurement of a sample saturated in water with excess solid.

Certain X-ray powder diffraction peaks for Form I of Compound 1 mesylate salt are shown in Table 8 below.

TABLE 8

| Pos. (° 2θ) | Rel. Int. (%) |
|---|---|
| 6.51 | 84.12 |
| 12.05 | 13.40 |
| 12.95 | 100.00 |
| 15.50 | 1.07 |
| 16.44 | 41.42 |
| 17.42 | 5.26 |
| 18.55 | 9.00 |
| 19.12 | 17.09 |
| 19.42 | 12.22 |
| 20.19 | 21.24 |
| 20.31 | 20.81 |
| 21.22 | 84.77 |
| 21.71 | 26.06 |
| 21.93 | 23.09 |
| 23.56 | 17.99 |
| 24.13 | 20.44 |
| 25.63 | 14.01 |
| 25.96 | 23.35 |
| 26.84 | 3.37 |
| 28.08 | 12.10 |
| 29.50 | 13.76 |
| 30.69 | 1.84 |
| 31.25 | 4.99 |
| 31.71 | 13.98 |
| 32.57 | 40.81 |
| 32.90 | 8.32 |
| 33.32 | 4.32 |
| 34.17 | 7.34 |
| 35.49 | 1.27 |
| 36.43 | 2.54 |
| 36.66 | 2.46 |
| 37.52 | 1.95 |
| 37.91 | 3.42 |
| 38.65 | 4.80 |
| 39.30 | 2.63 |

One aspect of the present invention is directed to a Compound 1 mesylate salt having an X-ray powder diffraction pattern comprising a peak, in terms of 2θ, at about 12.95°. In some embodiments, the salt has an X-ray powder diffraction pattern comprising a peak, in terms of 2θ, at about 21.22°. In some embodiments, the salt has an X-ray powder diffraction pattern comprising peaks, in terms of 2θ at about 12.95° and about 21.22°. In some embodiments, the salt has an X-ray powder diffraction pattern comprising peaks, in terms of 2θ, at about 12.95° and about 6.51°. In some embodiments, the salt has an X-ray powder diffraction pattern comprising peaks, in terms of 2θ at about 12.95°, about 21.22°, and about 6.51°. In some embodiments, the salt has an X-ray powder diffraction pattern comprising peaks, in terms of 2θ, at about 12.95°, about 21.22°, about 6.51°, about 16.44°, about 32.57°, about 21.71°, and about 25.96°. In some embodiments, the salt has an X-ray powder diffraction pattern comprising peaks, in terms of 2θ at about 12.95°, about 21.22°, about 6.51°, about 16.44°, about 32.57°, about 21.71°, about 25.96°, about 21.93°, about 20.19°, and about 20.31°. One aspect of the present invention is directed to a Compound 1 mesylate salt having an X-ray powder diffraction pattern comprising one or more peaks listed in Table 8. In some embodiments, the salt has an X-ray powder diffraction pattern substantially as shown in FIG. 12, wherein by “substantially” is meant that the reported peaks can vary by about ±0.2 °2θ and also that the relative intensities of the reported peaks can vary.

In some embodiments, the Compound 1 mesylate salt has a differential scanning calorimetry thermogram comprising an endotherm with an extrapolated onset temperature between about 160° C. and about 190° C. In some embodiments, the Compound 1 mesylate salt has a differential scanning calorimetry thermogram comprising an endotherm with an extrapolated onset temperature at about 178° C. In some embodiments, the Compound 1 mesylate salt has a differential scanning calorimetry thermogram comprising an endotherm with an associated heat flow of about 116 joules per gram. In some embodiments, the Compound 1 mesylate salt has a thermogravimetric analysis profile substantially as shown in FIG. 13, wherein by “substantially” is meant that the reported TGA features can vary by about ±5° C. and by about ±2% weight change.

In some embodiments, the Compound 1 mesylate salt has a differential scanning calorimetry thermogram substantially as shown in FIG. 13, wherein by “substantially” is meant that the reported DSC features can vary by about ±6° C. and by about ±20 joules per gram.

In some embodiments, the Compound 1 mesylate salt has a dynamic moisture sorption profile substantially as shown in FIG. 14, wherein by “substantially” is meant that the reported DMS features can vary by about ±5% relative humidity and by about ±5% weight change.

Form I of Compound 1 mesylate salt can be prepared by any of the suitable procedures known in the art for preparing crystalline polymorphs. In some embodiments Form I of Compound 1 mesylate salt can be prepared as described in Example 3. In some embodiments, Form I of Compound 1 mesylate salt can be prepared by slurrying crystalline Compound 1 mesylate salt containing one or more crystalline forms other than Form I. In some embodiments, the Compound 1 mesylate salt can be prepared by recrystallizing crystalline Compound 1 mesylate salt containing one or more crystalline forms other than Form I.

### Compound 1 Hydrobromide Salt Hemihydrate

One aspect of the present invention pertains to (R)-8-chloro-1-methyl-2,3,4,5-tetrahydro-1H-3-benzazepine hydrobromide salt hemihydrate, Form I (Compound 1 hydrobromide salt hemihydrate, Form I). The physical properties of Compound 1 hydrobromide salt hemihydrate, Form I are summarized in Table 9 below.

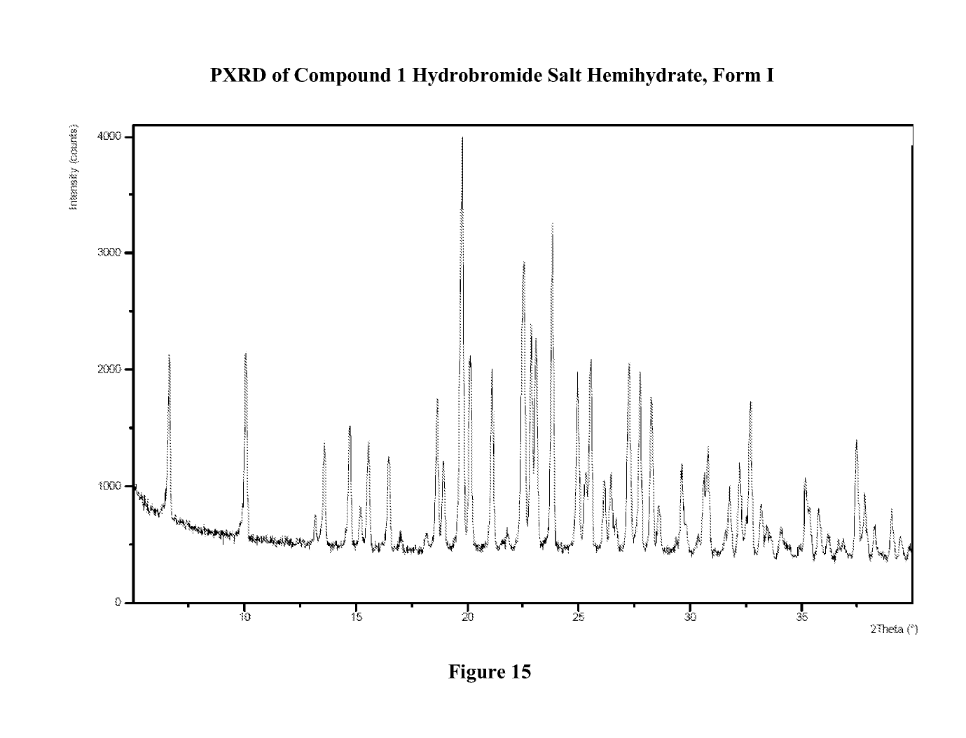

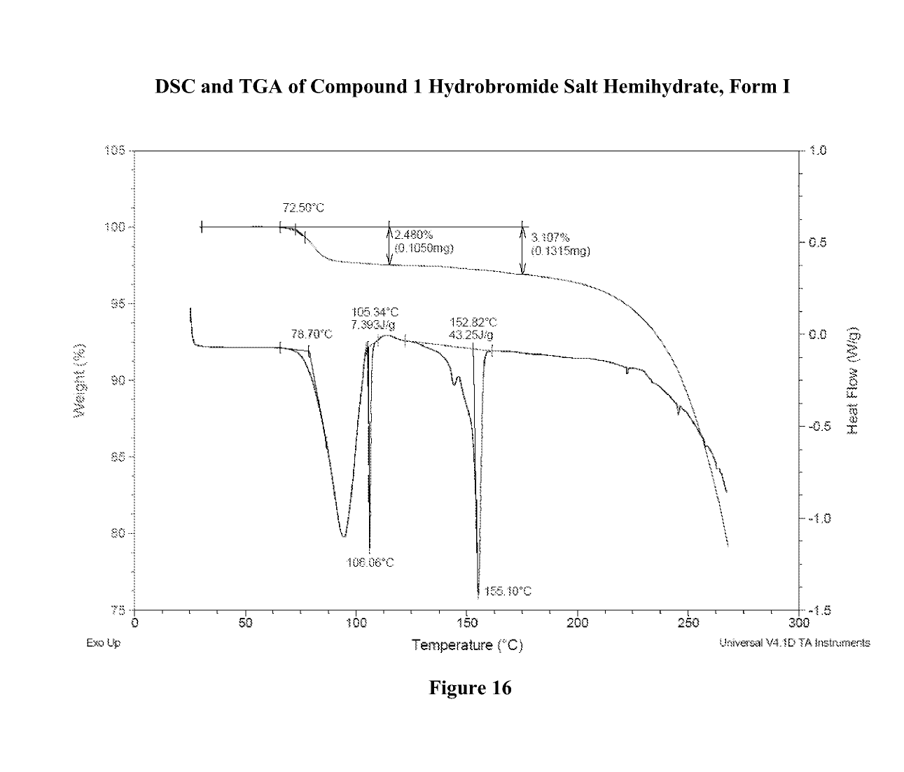

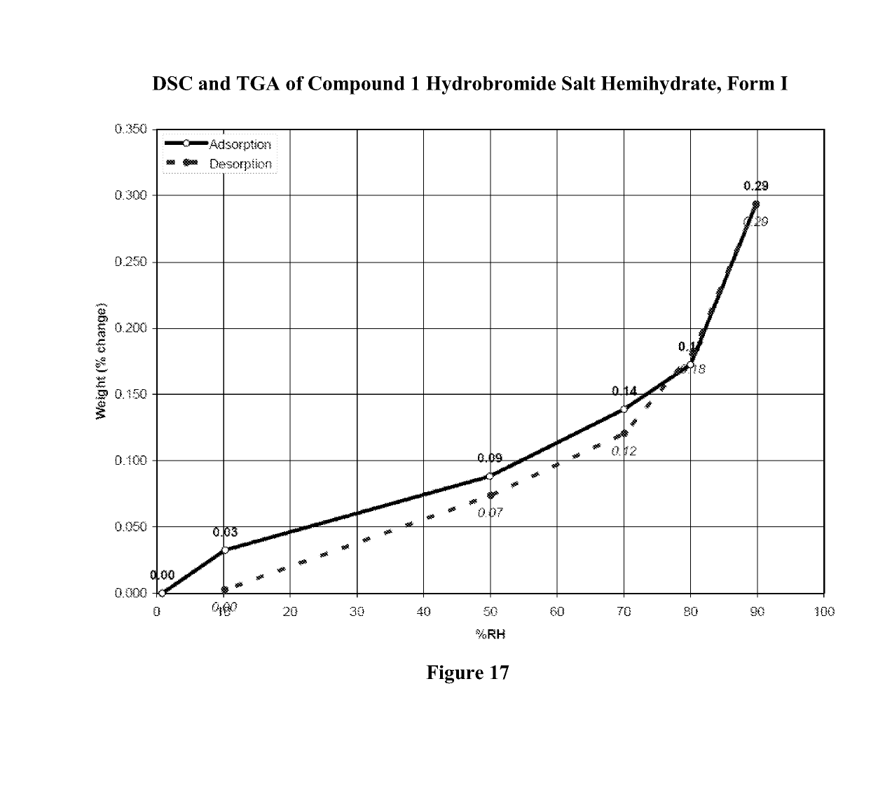

TABLE 9

| | Compound 1 Hydrobromide Salt Hemihydrate, Form I |
|---|---|
| PXRD | FIG. 15: Peaks of ≧40% relative intensity at |
| | 10.06, 19.77, 20.14, 21.12, 22.54, 22.87, 23.09, 23.82, |
| | 24.95, 25.54, 27.26, and 27.76° 2θ |
| TGA | FIG. 16: dehydration onset at about 72.5° C. |
| DMS | FIG. 17: ~0.3% weight at 90% RH |

Compound 1 hydrobromide salt, Form I was a hemihydrate with a dehydration onset at about 72.5° C. by TGA. The water content was lower than the theoretical value for a hemihydrate (3.15%) when the TGA integration was carried out to the perceived end of the DSC dehydration endotherm. An upper integration limit of about ˜175° C. was needed to achieve a weight loss equivalent to 0.5 moles of water. Karl Fischer titration was used to confirm the water content to be 3.18±0.04%.

Form I was non-hygroscopic, picking up ˜0.3% weight out to and including the 90% RH hold at 25° C. Analysis of a saturated aqueous solution with excess solid by water activity meter showed a very high DRH of 98% RH at 25° C.

Certain X-ray powder diffraction peaks for Form I of Compound 1 hydrobromide salt hemihydrate are shown in Table 10 below.

TABLE 10

| Pos. (°2θ) | Rel. Int. (%) |
|---|---|
| 6.62 | 38.40 |
| 10.06 | 45.37 |
| 13.19 | 7.20 |
| 13.59 | 24.38 |
| 14.73 | 28.94 |
| 15.21 | 9.53 |
| 15.56 | 25.09 |
| 16.48 | 22.27 |
| 17.02 | 3.72 |
| 18.15 | 3.75 |
| 18.65 | 36.58 |
| 18.93 | 20.92 |
| 19.77 | 100.00 |
| 20.14 | 45.49 |
| 21.12 | 42.61 |
| 21.82 | 3.71 |
| 22.54 | 69.31 |
| 22.87 | 53.27 |
| 23.09 | 50.58 |
| 23.82 | 78.48 |
| 24.95 | 42.42 |
| 25.32 | 18.68 |
| 25.54 | 43.96 |
| 26.16 | 16.35 |
| 26.44 | 18.29 |
| 26.68 | 7.29 |
| 27.26 | 44.48 |
| 27.76 | 40.55 |
| 28.27 | 36.03 |
| 28.60 | 11.00 |
| 29.64 | 21.62 |
| 30.62 | 17.12 |
| 30.80 | 25.39 |
| 31.77 | 16.61 |
| 32.22 | 21.27 |
| 32.70 | 36.23 |
| 33.19 | 12.14 |
| 33.45 | 6.53 |
| 33.58 | 5.03 |
| 34.10 | 6.47 |
| 35.18 | 19.22 |
| 35.40 | 9.18 |
| 35.77 | 11.75 |
| 36.21 | 5.12 |
| 36.68 | 3.98 |
| 36.89 | 4.05 |
| 37.48 | 27.20 |
| 37.85 | 15.59 |
| 38.28 | 7.16 |
| 39.05 | 11.17 |
| 39.44 | 5.24 |

One aspect of the present invention is directed to a Compound 1 hydrobromide salt hemihydrate having an X-ray powder diffraction pattern comprising a peak, in terms of 2θ, at about 19.77°. In some embodiments, the salt has an X-ray powder diffraction pattern comprising a peak, in terms of 2θ, at about 23.82°. In some embodiments, the salt has an X-ray powder diffraction pattern comprising peaks, in terms of 2θ, at about 19.77° and about 23.82°. In some embodiments, the salt has an X-ray powder diffraction pattern comprising peaks, in terms of 2θ, at about 19.77° and about 22.54°. In some embodiments, the salt has an X-ray powder diffraction pattern comprising peaks, in terms of 2θ, at about 19.77°, about 23.82°, and about 22.54°. In some embodiments, the salt has an X-ray powder diffraction pattern comprising peaks, in terms of 2θ, at about 19.77°, about 23.82°, about 22.54°, about 22.87°, about 23.09°, about 20.14°, and about 10.06°. In some embodiments, the salt has an X-ray powder diffraction pattern comprising peaks, in terms of 2θ, at about 19.77°, about 23.82°, about 22.54°, about 22.87°, about 23.09°, about 20.14°, about 10.06°, about 27.26°, about 25.54°, and about 20.31°. One aspect of the present invention is directed to a Compound 1 hydrobromide salt hemihydrate having an X-ray powder diffraction pattern comprising one or more peaks listed in Table 10. In some embodiments, the salt has an X-ray powder diffraction pattern substantially as shown in FIG. 15, wherein by “substantially” is meant that the reported peaks can vary by about ±0.2 °2θ and also that the relative intensities of the reported peaks can vary.

In some embodiments, the Compound 1 hydrobromide salt hemihydrate has a thermogravimetric analysis profile substantially as shown in FIG. 16, wherein by “substantially” is meant that the reported TGA features can vary by about ±5° C. and by about ±2% weight change.

In some embodiments, the Compound 1 hydrobromide salt hemihydrate has a differential scanning calorimetry thermogram substantially as shown in FIG. 16, wherein by “substantially” is meant that the reported DSC features can vary by about ±6° C. and by about ±20 joules per gram.

In some embodiments, the Compound 1 hydrobromide salt hemihydrate has a dynamic moisture sorption profile substantially as shown in FIG. 17, wherein by “substantially” is meant that the reported DMS features can vary by about ±5% relative humidity and by about ±5% weight change.

Form I of Compound 1 hydrobromide salt hemihydrate can be prepared by any of the suitable procedures known in the art for preparing crystalline polymorphs. In some embodiments Form I of Compound 1 hydrobromide salt hemihydrate can be prepared as described in Example 4. In some embodiments, Form I of Compound 1 hydrobromide salt hemihydrate can be prepared by slurrying crystalline Compound 1 hydrobromide salt hemihydrate containing one or more crystalline forms other than Form I. In some embodiments, the Compound 1 hydrobromide salt hemihydrate salt can be prepared by recrystallizing crystalline Compound 1 hydrobromide salt hemihydrate salt containing one or more crystalline forms other than Form I.

### Compound 1 Nitrate Salt

One aspect of the present invention pertains to (R)-8-chloro-1-methyl-2,3,4,5-tetrahydro-1H-3-benzazepine nitrate salt, Form I (Compound 1 nitrate salt, Form I). The physical properties of Compound 1 nitrate salt, Form I are summarized in Table 11 below.

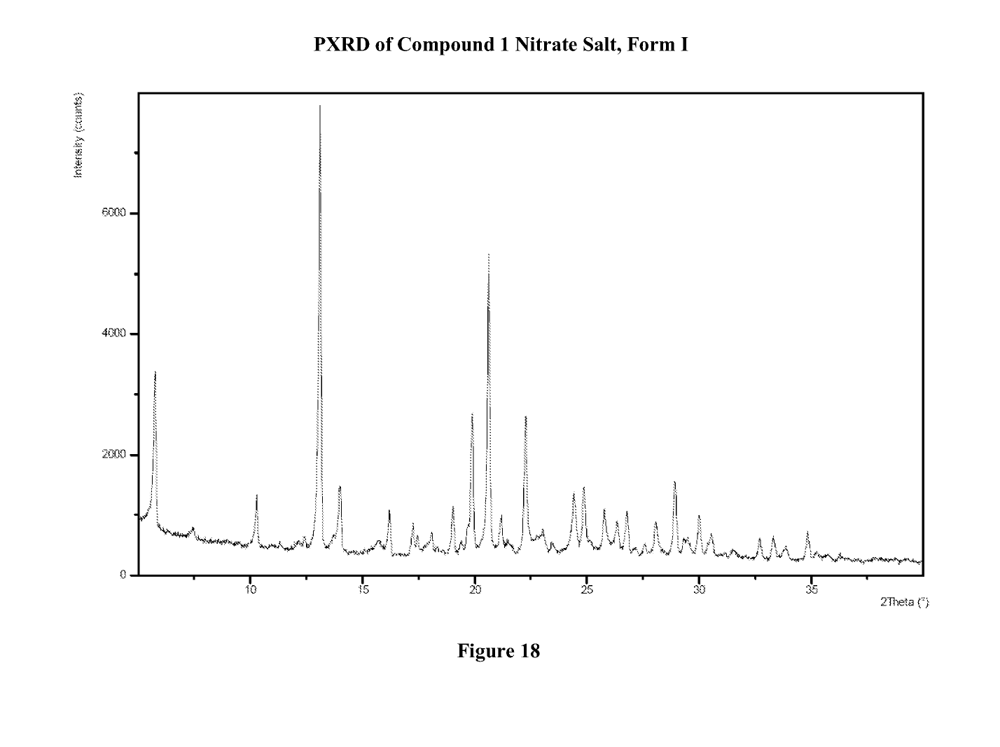

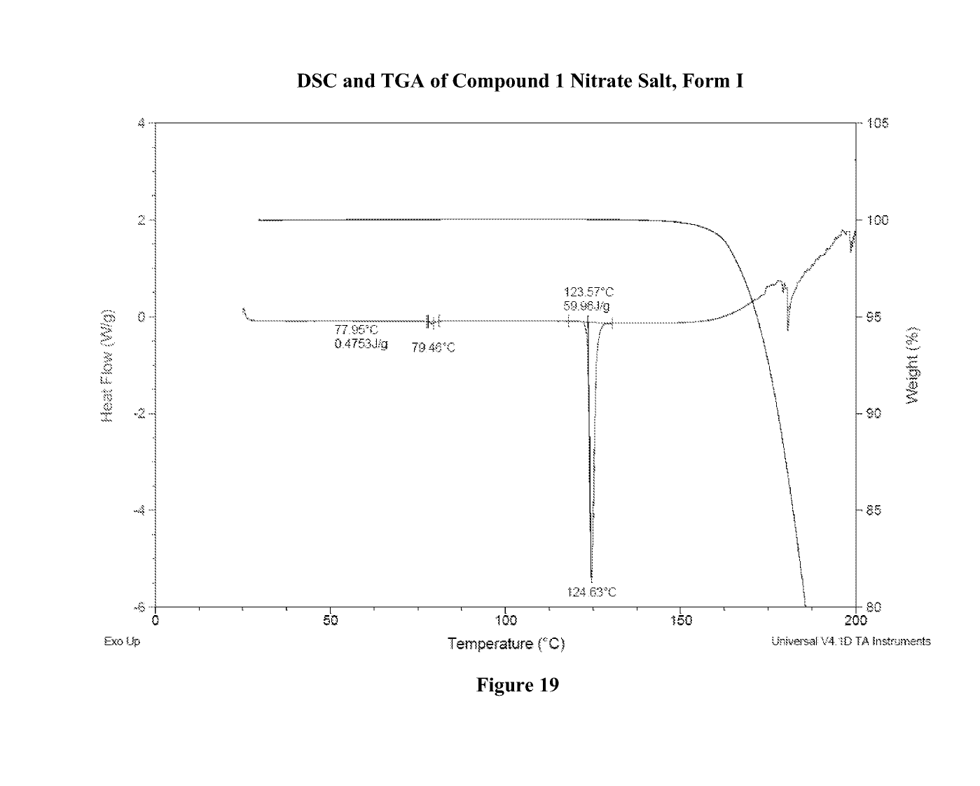

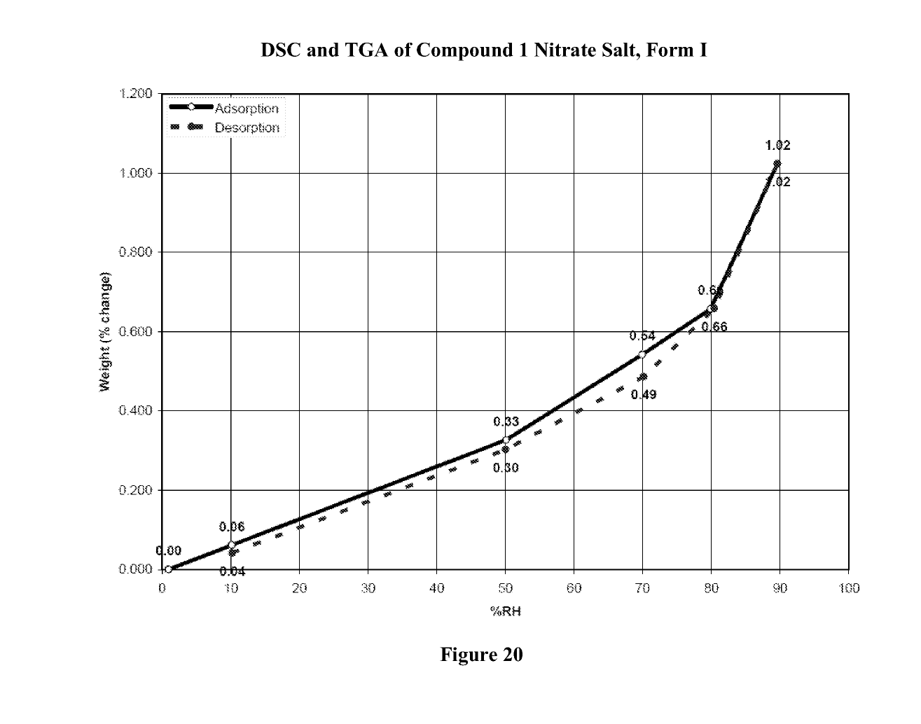

TABLE 11

| | Compound 1 Nitrate Salt, Form I |
|---|---|
| PXRD | FIG. 18: Peaks of ≧10% relative intensity at 13.10, 20.62, |
| | 5.75, 19.88, 22.27, 28.92, 24.86, 13.99, 24.43, 10.28, 19.02, |
| | 25.77, 16.19, and 26.79 °2θ |
| TGA | FIG. 19: >1% weight loss up to about 150° C. |
| DSC | FIG. 19: extrapolated onset temperature about 124° C.; |
| | enthalpy of fusion 60 J/g |
| DMS | FIG. 20: ~1% weight gained out to 90% RH |

Form I of Compound 1 nitrate salt was an anhydrous material with a melting onset of about 124° C. The title salt was very slightly hygroscopic, picking up ˜1% weight by DMS analysis out to and including the 90% RH hold at 25° C. The DRH by water activity measurement of a saturated solution with excess solid was 99% RH at 25° C.

Certain X-ray powder diffraction peaks for Form I of Compound 1 nitrate salt are shown in Table 12 below.

TABLE 12

| Pos. (°2θ) | Rel. Int. (%) |
|---|---|
| 5.75 | 33.39 |
| 7.44 | 2.73 |
| 10.28 | 11.40 |
| 11.32 | 1.54 |
| 12.12 | 1.99 |
| 12.43 | 3.18 |
| 13.10 | 100.00 |
| 13.99 | 14.85 |
| 15.72 | 3.45 |
| 16.19 | 10.18 |
| 17.24 | 6.98 |
| 17.44 | 4.52 |
| 18.08 | 5.39 |
| 18.32 | 2.03 |
| 19.02 | 11.01 |
| 19.38 | 2.91 |
| 19.66 | 5.88 |
| 19.88 | 31.98 |
| 20.62 | 67.38 |
| 21.18 | 8.81 |
| 21.48 | 3.43 |
| 22.27 | 31.27 |
| 23.03 | 5.99 |
| 23.45 | 2.84 |
| 24.43 | 13.16 |
| 24.86 | 15.50 |
| 25.77 | 10.55 |
| 26.35 | 7.81 |
| 26.79 | 10.11 |
| 27.13 | 1.80 |
| 27.58 | 2.98 |
| 28.07 | 7.77 |
| 28.92 | 16.88 |
| 29.32 | 4.01 |
| 29.47 | 4.29 |
| 30.01 | 9.46 |
| 30.55 | 5.62 |
| 31.52 | 2.01 |
| 32.69 | 4.87 |
| 33.31 | 4.77 |
| 33.86 | 3.11 |
| 34.84 | 6.81 |
| 35.23 | 1.96 |
| 35.70 | 1.45 |
| 36.26 | 1.71 |
| 37.95 | 0.97 |
| 38.69 | 0.87 |
| 39.21 | 0.99 |

One aspect of the present invention is directed to a Compound 1 nitrate salt having an X-ray powder diffraction pattern comprising a peak, in terms of 2θ, at about 5.75°. In some embodiments, the salt has an X-ray powder diffraction pattern comprising a peak, in terms of 2θ, at about 10.28°. In some embodiments, the salt has an X-ray powder diffraction pattern comprising peaks, in terms of 2θ at about 5.75° and about 10.28°. In some embodiments, the salt has an X-ray powder diffraction pattern comprising peaks, in terms of 2θ, at about 5.75° and about 13.10°. In some embodiments, the salt has an X-ray powder diffraction pattern comprising peaks, in terms of 2θ at about 5.75°, about 10.28°, and about 13.10°. In some embodiments, the salt has an X-ray powder diffraction pattern comprising peaks, in terms of 2θ, at about 5.75°, about 10.28°, about 13.10°, about 13.99°, about 16.19°, about 19.02°, and about 19.88°. In some embodiments, the salt has an X-ray powder diffraction pattern comprising peaks, in terms of 2θ at about 5.75°, about 10.28°, about 13.10°, about 13.99°, about 16.19°, about 19.02°, about 19.88°, about 20.62°, about 22.27°, and about 24.43°. One aspect of the present invention is directed to a Compound 1 nitrate salt having an X-ray powder diffraction pattern comprising one or more peaks listed in Table 12. In some embodiments, the salt has an X-ray powder diffraction pattern substantially as shown in FIG. 18, wherein by “substantially” is meant that the reported peaks can vary by about ±0.2 °2θ, and also that the relative intensities of the reported peaks can vary.

In some embodiments, the Compound 1 nitrate salt has a differential scanning calorimetry thermogram comprising an endotherm with an extrapolated onset temperature between about 110° C. and about 140° C. In some embodiments, the Compound 1 nitrate salt has a differential scanning calorimetry thermogram comprising an endotherm with an extrapolated onset temperature at about 120° C. In some embodiments, the Compound 1 nitrate salt has a differential scanning calorimetry thermogram comprising an endotherm with an associated heat flow of about 60 joules per gram. In some embodiments, the Compound 1 nitrate salt has a thermogravimetric analysis profile substantially as shown in FIG. 19, wherein by “substantially” is meant that the reported TGA features can vary by about ±5° C. and by about ±2% weight change.

In some embodiments, the Compound 1 nitrate salt has a differential scanning calorimetry thermogram substantially as shown in FIG. 19, wherein by “substantially” is meant that the reported DSC features can vary by about ±6° C. and by about ±20 joules per gram.

In some embodiments, the Compound 1 nitrate salt has a dynamic moisture sorption profile substantially as shown in FIG. 20, wherein by “substantially” is meant that the reported DMS features can vary by about ±5% relative humidity and by about ±5% weight change.

Form I of Compound 1 nitrate salt can be prepared by any of the suitable procedures known in the art for preparing crystalline polymorphs. In some embodiments Form I of Compound 1 nitrate salt can be prepared as described in Example 5. In some embodiments, Form I of Compound 1 nitrate salt can be prepared by slurrying crystalline Compound 1 nitrate salt containing one or more crystalline forms other than Form I. In some embodiments, Form I of Compound 1 nitrate salt can be prepared by recrystallizing crystalline Compound 1 nitrate salt containing one or more crystalline forms other than Form I.

### Compound 1 Sesqui-oxalate Salt-Cocrystal

One aspect of the present invention pertains to (R)-8-chloro-1-methyl-2,3,4,5-tetrahydro-1H-3-benzazepine sesqui-oxalate salt-cocrystal, Form I (Compound 1 sesqui-oxalate salt-cocrystal, Form I). The physical properties of Compound 1 sesqui-oxalate salt-cocrystal, Form I are summarized in Table 13 below.

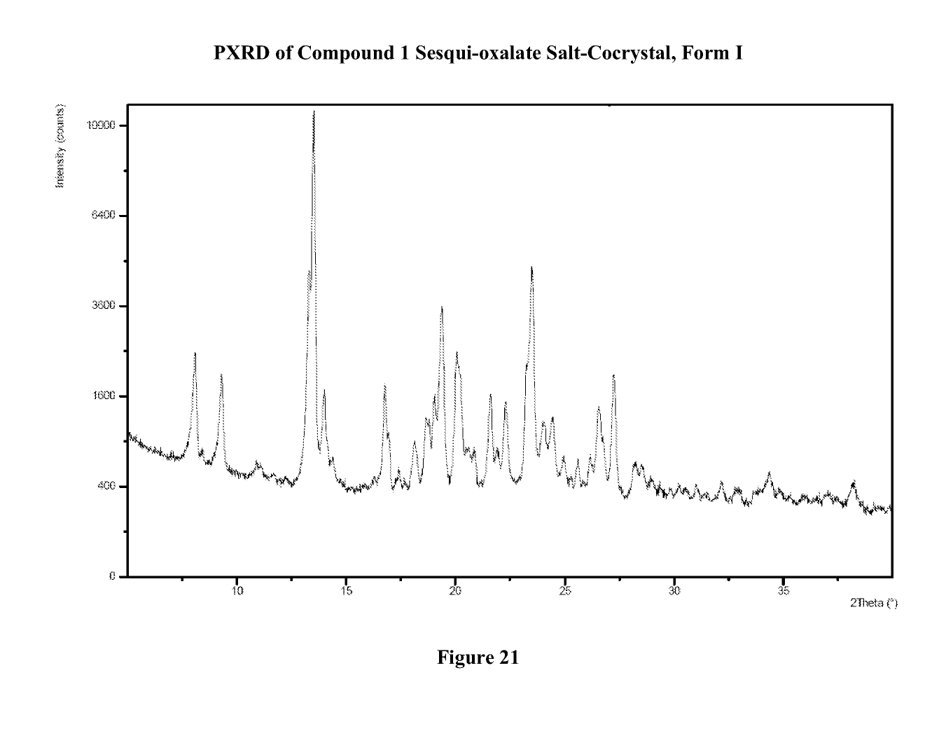

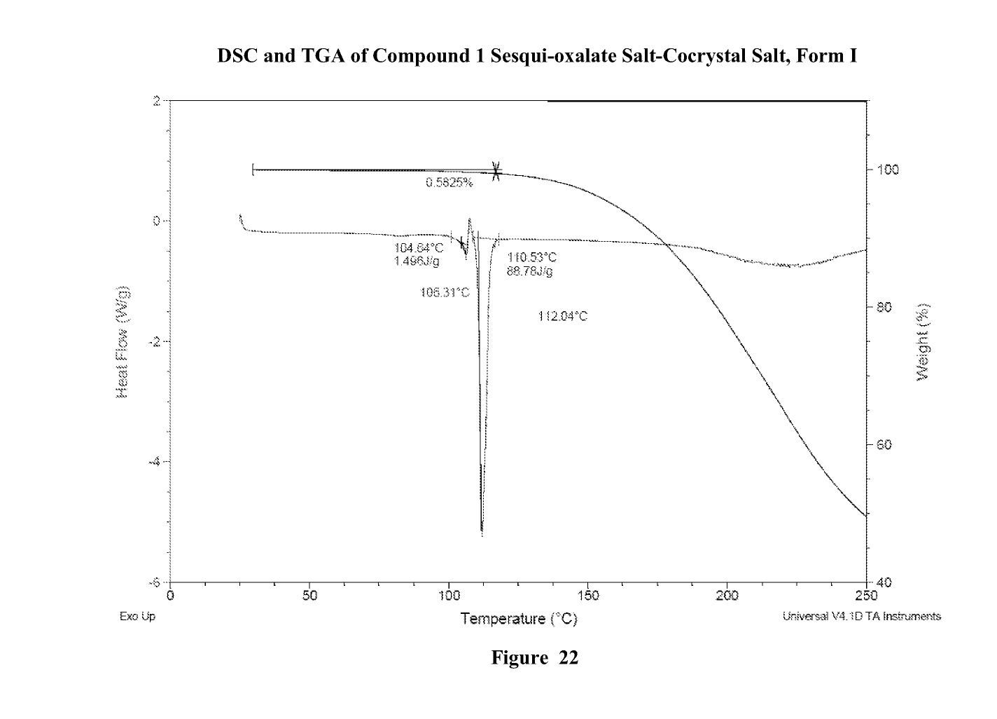

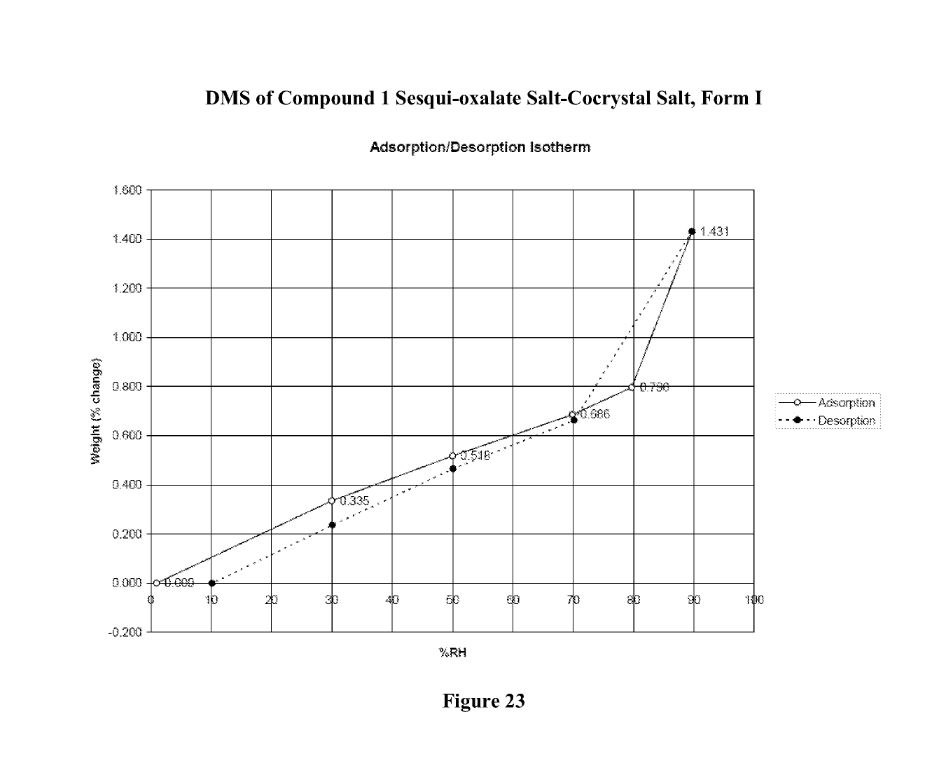

TABLE 13

| | Compound 1 Sesqui-oxalate Salt-Cocrystal, Form I |
|---|---|
| PXRD | FIG. 21: Peaks of ≧12% relative intensity at 8.09, 9.31, |
| | 13.31, 13.52, 14.00, 16.77, 19.04, 19.38, 20.06, 20.23, |
| | 21.61, 23.23, 23.50, and 27.24 °2θ |
| TGA | FIG. 22: less than 0.6% weight loss up to about 115° C. |
| DSC | FIG. 22: extrapolated onset temperatures at about 105° C. |
| | and at about 111° C. with an enthalpy of fusion of about 89 |
| | J/g for the latter |
| DMS | FIG. 23: about 1.4% weight gain at 90% RH |

Form I of Compound 1 sesqui-oxalate salt showed by DSC an apparent melt, followed immediately by recrystallization, and followed immediately by melting. The initial endotherm has an onset of 105° C.; the second endotherm has a melting onset of 111° C. The title salt was slightly hygroscopic, picking up about 1.4% weight out to and including the 90% RH hold at 25° C.

Certain X-ray powder diffraction peaks for Form I of Compound 1 sesqui-oxalate salt-cocrystal are shown in Table 14 below.

TABLE 14

| Pos. (°2θ) | Rel. Int. (%) |
|---|---|
| 8.09 | 18.34 |
| 8.41 | 1.76 |
| 9.31 | 13.98 |
| 10.99 | 1.34 |
| 11.67 | 0.96 |
| 13.31 | 41.02 |
| 13.52 | 100.00 |
| 14.00 | 12.91 |
| 14.38 | 3.13 |
| 16.30 | 1.41 |
| 16.77 | 14.42 |
| 17.41 | 2.15 |
| 18.13 | 5.27 |
| 18.68 | 8.70 |
| 19.04 | 12.32 |
| 19.38 | 31.31 |
| 20.06 | 20.96 |
| 20.23 | 15.75 |
| 20.58 | 4.45 |
| 20.87 | 4.77 |
| 21.61 | 12.78 |
| 21.91 | 4.82 |
| 22.30 | 11.38 |
| 23.23 | 16.20 |
| 23.50 | 42.75 |
| 24.0430 | 8.29 |
| 24.4477 | 8.75 |
| 24.9665 | 3.95 |
| 25.3023 | 1.85 |
| 25.6034 | 3.51 |
| 26.1744 | 4.18 |
| 26.5544 | 10.70 |
| 27.24 | 16.81 |
| 28.13 | 2.93 |
| 28.54 | 3.26 |
| 28.98 | 1.88 |
| 29.83 | 1.08 |
| 30.23 | 1.42 |
| 30.46 | 0.91 |
| 31.02 | 1.51 |
| 32.18 | 1.90 |
| 32.90 | 1.27 |
| 33.73 | 1.16 |
| 34.36 | 2.98 |
| 35.95 | 0.97 |
| 37.02 | 1.30 |
| 38.20 | 2.13 |

One aspect of the present invention is directed to a Compound 1 sesqui-oxalate salt-cocrystal having an X-ray powder diffraction pattern comprising a peak, in terms of 2θ, at about 13.52°. In some embodiments, the salt has an X-ray powder diffraction pattern comprising a peak, in terms of 2θ, at about 23.50°. In some embodiments, the salt has an X-ray powder diffraction pattern comprising peaks, in terms of 2θ, at about 13.52° and about 23.50°. In some embodiments, the salt has an X-ray powder diffraction pattern comprising peaks, in terms of 2θ, at about 13.52° and about 13.31°. In some embodiments, the salt has an X-ray powder diffraction pattern comprising peaks, in terms of 2θ, at about 13.52°, about 23.50°, and about 13.31°. In some embodiments, the salt has an X-ray powder diffraction pattern comprising peaks, in terms of 2θ, at about 13.52°, about 23.50°, about 13.31°, about 19.38°, about 20.06°, about 8.09°, and about 27.24°. In some embodiments, the salt has an X-ray powder diffraction pattern comprising peaks, in terms of 2θ, at about 13.52°, about 23.50°, about 13.31°, about 19.38°, about 20.06°, about 8.09°, about 27.24°, about 23.23°, about 20.23°, and about 16.77°. One aspect of the present invention is directed to a Compound 1 sesqui-oxalate salt-cocrystal having an X-ray powder diffraction pattern comprising one or more peaks listed in Table 14. In some embodiments, the salt has an X-ray powder diffraction pattern substantially as shown in FIG. 21, wherein by “substantially” is meant that the reported peaks can vary by about ±0.2 °2θ and also that the relative intensities of the reported peaks can vary.

In some embodiments, the Compound 1 sesqui-oxalate salt-cocrystal has a differential scanning calorimetry thermogram comprising an endotherm with an extrapolated onset temperature between about 90° C. and about 120° C. In some embodiments, the Compound 1 sesqui-oxalate salt-cocrystal has a differential scanning calorimetry thermogram comprising an endotherm with an extrapolated onset temperature at about 105° C.

In some embodiments, the Compound 1 sesqui-oxalate salt-cocrystal has a differential scanning calorimetry thermogram comprising an endotherm with an extrapolated onset temperature between about 95° C. and about 125° C. In some embodiments, the Compound 1 sesqui-oxalate salt-cocrystal has a differential scanning calorimetry thermogram comprising an endotherm with an extrapolated onset temperature at about 111° C.

In some embodiments, the Compound 1 sesqui-oxalate salt-cocrystal has a differential scanning calorimetry thermogram comprising an endotherm with an extrapolated onset temperature between about 90° C. and about 120° C., and an endotherm with an extrapolated onset temperature between about 95° C. and about 125° C. In some embodiments, the Compound 1 sesqui-oxalate salt-cocrystal has a differential scanning calorimetry thermogram comprising an endotherm with an extrapolated onset temperature at about 105° C., and an endotherm with an extrapolated onset temperature at about 111° C. In some embodiments, the Compound 1 sesqui-oxalate salt-cocrystal has a differential scanning calorimetry thermogram comprising an endotherm with an associated heat flow of about 89 joules per gram. In some embodiments, the Compound 1 sesqui-oxalate salt-cocrystal has a thermogravimetric analysis profile substantially as shown in FIG. 22, wherein by “substantially” is meant that the reported TGA features can vary by about ±5° C. and by about ±2% weight change.

In some embodiments, the Compound 1 sesqui-oxalate salt-cocrystal has a differential scanning calorimetry thermogram substantially as shown in FIG. 22, wherein by “substantially” is meant that the reported DSC features can vary by about ±6° C. and by about ±20 joules per gram.

In some embodiments, the Compound 1 sesqui-oxalate salt-cocrystal has a dynamic moisture sorption profile substantially as shown in FIG. 23, wherein by “substantially” is meant that the reported DMS features can vary by about ±5% relative humidity and by about ±5% weight change.

Form I of Compound 1 sesqui-oxalate salt-cocrystal can be prepared by any of the suitable procedures known in the art for preparing crystalline polymorphs. In some embodiments Form I of Compound 1 sesqui-oxalate salt-cocrystal can be prepared as described in Example 6. In some embodiments, Form I of Compound 1 sesqui-oxalate salt-cocrystal can be prepared by slurrying crystalline Compound 1 sesqui-oxalate salt-cocrystal containing one or more crystalline forms other than Form I. In some embodiments, Form I of Compound 1 sesqui-oxalate salt-cocrystal can be prepared by recrystallizing crystalline Compound 1 sesqui-oxalate salt-cocrystal containing one or more crystalline forms other than Form I.

### Compound 1 Adipate Salt.

One aspect of the present invention pertains to (R)-8-chloro-1-methyl-2,3,4,5-tetrahydro-1H-3-benzazepine adipate salt, Form I (Compound 1 adipate salt, Form I). The physical properties of Compound 1 adipate salt, Form I are summarized in Table 15 below.

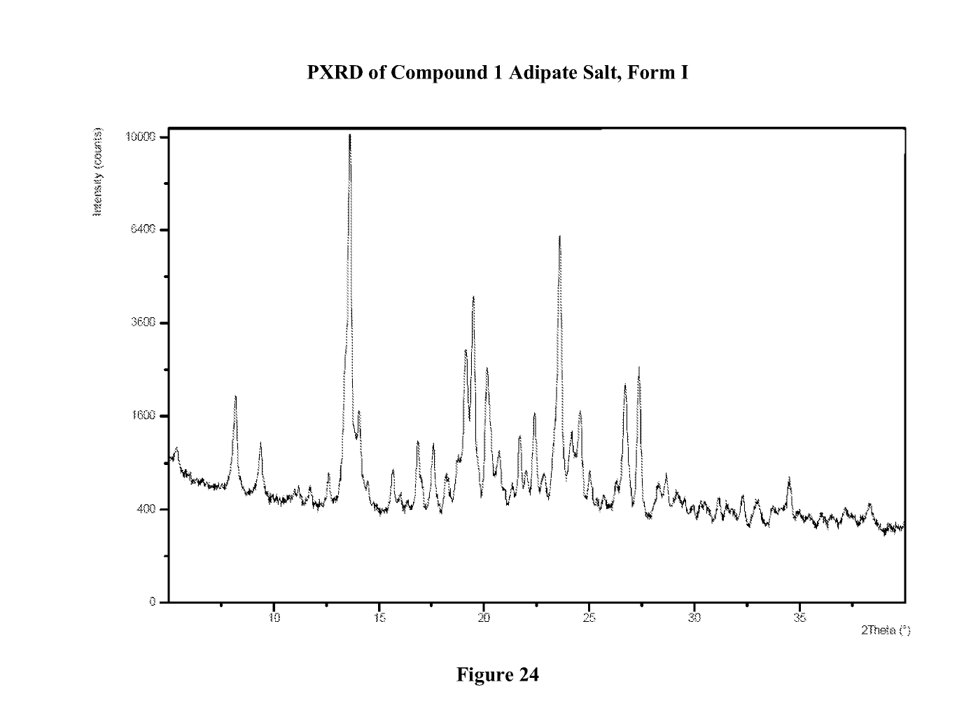

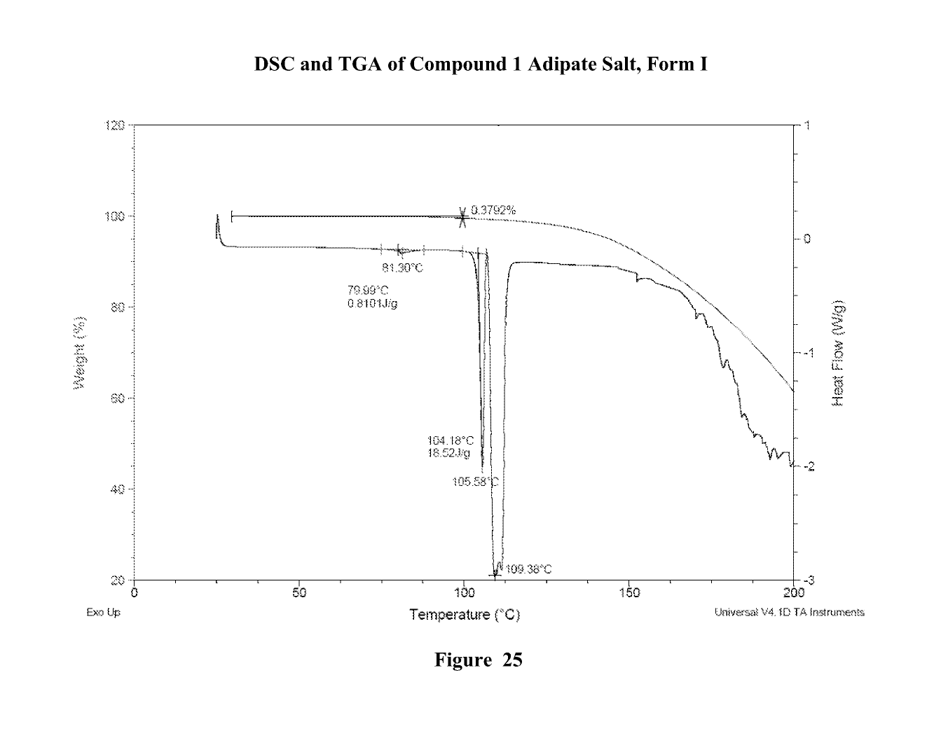

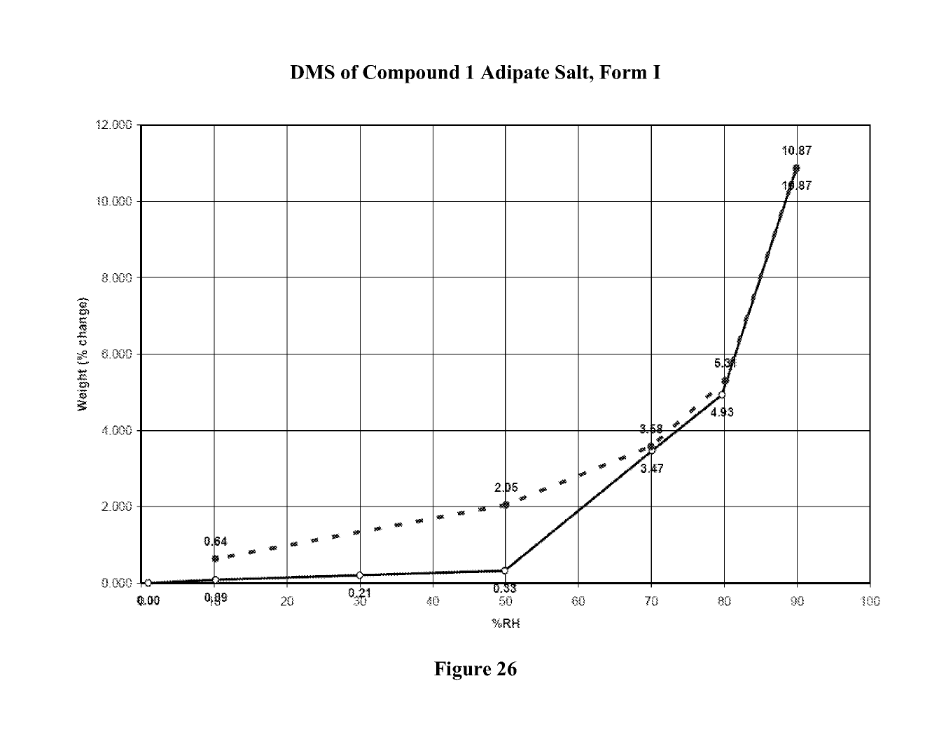

TABLE 15

| | Compound 1 Adipate Salt, Form I |
|---|---|
| PXRD | FIG. 24: Peaks of ≧10% relative intensity at 8.20, 13.39, |
| | 13.63, 14.07, 19.13, 19.49, 20.14, 22.40, 23.60, 24.57, |
| | 26.70, and 27.36 °2θ |
| TGA | FIG. 25: <0.4% weight loss up to about 100° C. |
| DSC | FIG. 25: multiple endothermic events |
| DMS | FIG. 26: 10.87% weight gain at 90% RH |

DSC and TGA analyses of Compound 1 adipate salt, Form I show that it was an anhydrous salt with multiple endothermic events. The larger and more closely spaced endotherms had onset temperatures of about 104° C. and 107° C. depending on the sample. The salt was hygroscopic at 70% RH and above, picking up 10.87% weight out to and including the 90% RH hold at 25° C.

Certain X-ray powder diffraction peaks for Form I of Compound 1 adipate salt are shown in Table 16 below.

TABLE 16

| Pos. (°2θ) | Rel. Int. (%) |
|---|---|
| 5.39 | 3.03 |
| 8.20 | 14.57 |
| 9.39 | 6.88 |
| 11.05 | 1.39 |
| 11.19 | 2.22 |
| 11.74 | 2.08 |
| 12.63 | 3.95 |
| 13.39 | 22.94 |
| 13.63 | 100.00 |
| 14.07 | 13.52 |
| 14.47 | 3.15 |
| 15.67 | 4.70 |
| 16.03 | 1.86 |
| 16.36 | 1.24 |
| 16.86 | 8.93 |
| 17.07 | 3.21 |
| 17.59 | 8.42 |
| 18.20 | 4.06 |
| 18.77 | 6.80 |
| 19.13 | 26.63 |
| 19.49 | 40.78 |
| 20.14 | 22.23 |
| 20.71 | 6.91 |
| 21.34 | 2.57 |
| 21.70 | 9.19 |
| 21.99 | 4.29 |
| 22.40 | 12.82 |
| 22.84 | 3.81 |
| 23.60 | 59.61 |
| 24.16 | 9.51 |
| 24.57 | 13.72 |
| 25.02 | 4.68 |
| 25.37 | 1.50 |
| 25.69 | 1.97 |
| 26.29 | 3.63 |
| 26.70 | 19.20 |
| 27.36 | 22.79 |
| 28.29 | 3.77 |
| 28.65 | 4.82 |
| 29.17 | 2.82 |
| 29.51 | 2.33 |
| 29.92 | 1.71 |
| 30.29 | 2.08 |
| 31.14 | 2.42 |
| 31.52 | 1.71 |
| 32.27 | 2.73 |
| 32.97 | 2.14 |
| 33.70 | 1.80 |
| 34.48 | 4.06 |
| 34.94 | 1.47 |
| 35.43 | 1.18 |
| 36.01 | 1.27 |
| 36.53 | 1.24 |
| 37.16 | 1.92 |
| 38.32 | 2.36 |
| 39.25 | 0.67 |

One aspect of the present invention is directed to a Compound 1 adipate salt having an X-ray powder diffraction pattern comprising a peak, in terms of 2θ, at about 13.63°. In some embodiments, the salt has an X-ray powder diffraction pattern comprising a peak, in terms of 2θ, at about 23.60°. In some embodiments, the salt has an X-ray powder diffraction pattern comprising peaks, in terms of 2θ, at about 13.63° and about 23.60°. In some embodiments, the salt has an X-ray powder diffraction pattern comprising peaks, in terms of 2θ, at about 13.63° and about 19.49°. In some embodiments, the salt has an X-ray powder diffraction pattern comprising peaks, in terms of 2θ, at about 13.63°, about 23.60°, and about 19.49°. In some embodiments, the salt has an X-ray powder diffraction pattern comprising peaks, in terms of 2θ, at about 13.63°, about 23.60°, about 19.49°, about 19.13°, about 13.39°, about 27.36°, and about 20.14°. In some embodiments, the salt has an X-ray powder diffraction pattern comprising peaks, in terms of 2θ, at about 13.63°, about 23.60°, about 19.49°, about 19.13°, about 13.39°, about 27.36°, about 20.14°, about 26.70°, about 8.20°, and about 24.57°. One aspect of the present invention is directed to a Compound 1 adipate salt having an X-ray powder diffraction pattern comprising one or more peaks listed in Table 16. In some embodiments, the salt has an X-ray powder diffraction pattern substantially as shown in FIG. 24, wherein by “substantially” is meant that the reported peaks can vary by about ±0.2 °2θ and also that the relative intensities of the reported peaks can vary.

In some embodiments, the Compound 1 adipate salt has a differential scanning calorimetry thermogram comprising an endotherm with an extrapolated onset temperature between about 90° C. and about 120° C. In some embodiments, the Compound 1 adipate salt has a differential scanning calorimetry thermogram comprising an endotherm with an extrapolated onset temperature at about 104° C. In some embodiments, the Compound 1 adipate salt has a differential scanning calorimetry thermogram comprising an endotherm with an associated heat flow of about 19 joules per gram. In some embodiments, the Compound 1 adipate salt has a thermogravimetric analysis profile substantially as shown in FIG. 25, wherein by “substantially” is meant that the reported TGA features can vary by about ±5° C. and by about ±2% weight change.

In some embodiments, the Compound 1 adipate salt has a differential scanning calorimetry thermogram substantially as shown in FIG. 25, wherein by “substantially” is meant that the reported DSC features can vary by about ±6° C. and by about ±20 joules per gram.

In some embodiments, the Compound 1 adipate salt has a dynamic moisture sorption profile substantially as shown in FIG. 26, wherein by “substantially” is meant that the reported DMS features can vary by about ±5% relative humidity and by about ±5% weight change.

Form I of Compound 1 adipate salt can be prepared by any of the suitable procedures known in the art for preparing crystalline polymorphs. In some embodiments Form I of Compound 1 adipate salt can be prepared as described in Example 7. In some embodiments, Form I of Compound 1 adipate salt can be prepared by slurrying crystalline Compound 1 adipate salt containing one or more crystalline forms other than Form I. In some embodiments, Form I of Compound 1 adipate salt can be prepared by recrystallizing crystalline Compound 1 adipate salt containing one or more crystalline forms other than Form I.

### Compound 1 Malonate Salt.

One aspect of the present invention pertains to (R)-8-chloro-1-methyl-2,3,4,5-tetrahydro-1H-3-benzazepine malonate salt, Form I (Compound 1 malonate salt, Form I). The physical properties of Compound 1 malonate salt, Form I are summarized in Table 17 below.

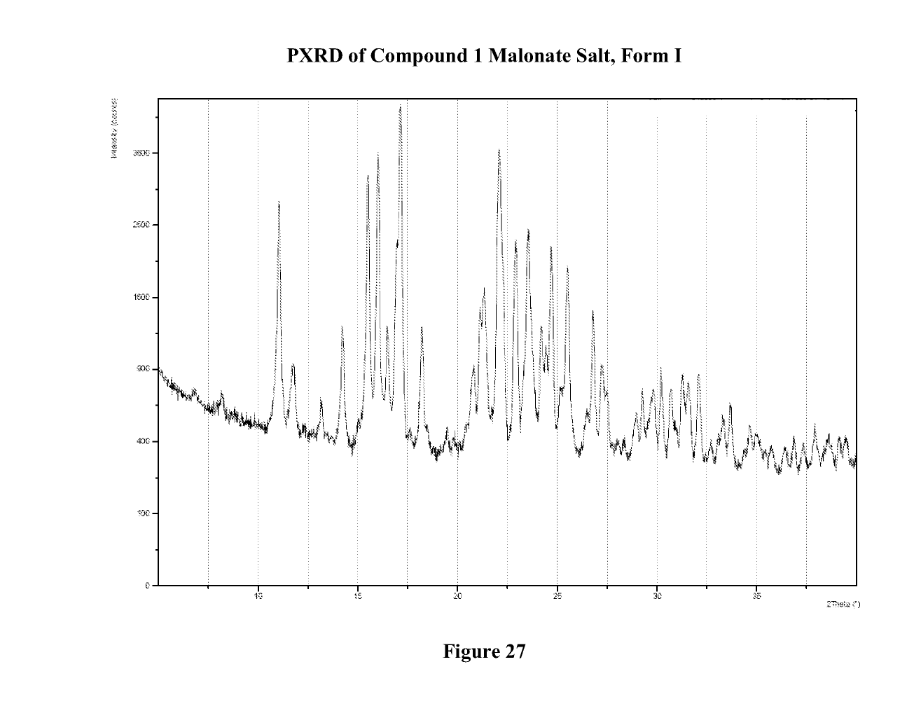

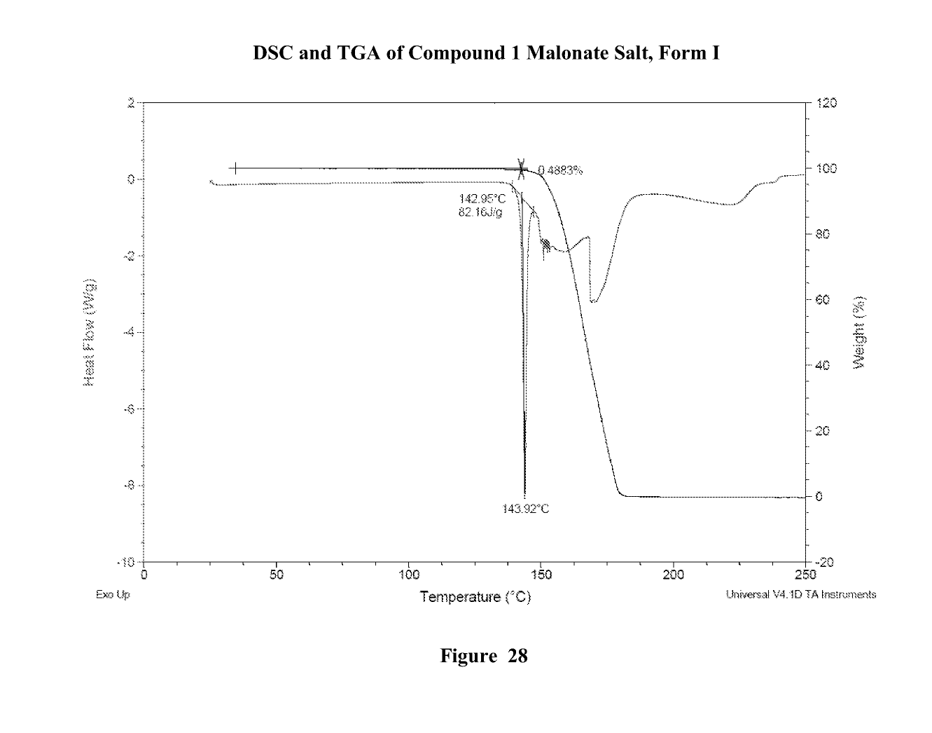

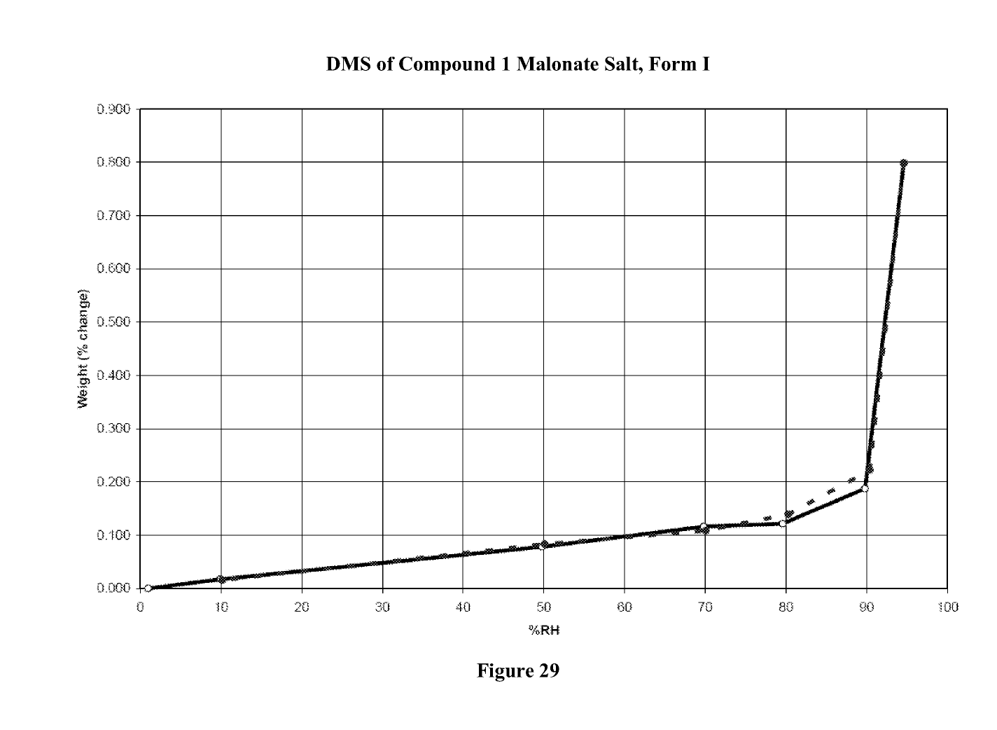

TABLE 17

| | Compound 1 Malonate Salt, Form I |
|---|---|
| PXRD | FIG. 27: Peaks of ≧25% relative intensity at 11.05, 15.51, |
| | 16.02, 16.97, 17.14, 21.13, 21.33, 22.08, 22.31, 22.91, |
| | 23.54, 24.70, 25.51, and 26.80 °2θ |
| TGA | FIG. 28: <0.5% up to about 145° C. |
| DSC | FIG. 28: extrapolated onset temperature about 143° C.; |
| | enthalpy of fusion about 82 J/g |
| DMS | FIG. 29: 0.2% weight gain at 90% RH |

Compound 1 malonate salt, Form I displayed a melting onset between about 143-145° C. The TGA showed complete volatilization of the salt after melting. It was non-hygroscopic, picking up ˜0.2% weight out to and including the 90% RH hold at 25° C.

Certain X-ray powder diffraction peaks for Form I of Compound 1 malonate salt are shown in Table 18 below.

TABLE 18

| Pos. (°2θ) | Rel. Int. (%) |
|---|---|
| 6.81 | 1.88 |
| 8.18 | 3.69 |
| 11.05 | 58.09 |
| 11.76 | 11.99 |
| 13.18 | 5.89 |
| 14.24 | 22.66 |
| 15.51 | 70.73 |
| 16.02 | 78.89 |
| 16.49 | 22.25 |
| 16.97 | 47.40 |
| 17.14 | 100.00 |
| 17.62 | 3.10 |
| 18.21 | 23.59 |
| 19.47 | 3.82 |
| 20.40 | 4.77 |
| 20.82 | 15.00 |
| 21.13 | 28.77 |
| 21.33 | 33.76 |
| 22.08 | 81.90 |
| 22.31 | 33.52 |
| 22.91 | 48.18 |
| 23.54 | 51.28 |
| 24.20 | 23.79 |
| 24.43 | 19.57 |
| 24.70 | 46.42 |
| 25.18 | 10.84 |
| 25.51 | 39.97 |
| 26.49 | 7.47 |
| 26.80 | 26.52 |
| 27.25 | 15.65 |
| 27.53 | 10.28 |
| 28.34 | 2.76 |
| 28.97 | 7.25 |
| 29.26 | 10.93 |
| 29.81 | 11.33 |
| 30.20 | 14.60 |
| 30.69 | 11.54 |
| 31.27 | 14.09 |
| 31.58 | 12.48 |
| 32.09 | 14.66 |
| 32.71 | 3.62 |
| 33.08 | 4.26 |
| 33.32 | 6.67 |
| 33.69 | 9.01 |
| 34.65 | 5.94 |
| 34.99 | 4.52 |
| 35.73 | 2.82 |
| 36.40 | 2.77 |
| 36.87 | 3.79 |
| 37.33 | 3.06 |
| 37.92 | 5.40 |
| 38.57 | 3.98 |
| 39.13 | 4.06 |
| 39.46 | 3.85 |

One aspect of the present invention is directed to a Compound 1 malonate salt having an X-ray powder diffraction pattern comprising a peak, in terms of 2θ, at about 17.14°. In some embodiments, the salt has an X-ray powder diffraction pattern comprising a peak, in terms of 20, at about 22.08°. In some embodiments, the salt has an X-ray powder diffraction pattern comprising peaks, in terms of 2θ, at about 17.14° and about 22.08°. In some embodiments, the salt has an X-ray powder diffraction pattern comprising peaks, in terms of 2θ, at about 17.14° and about 16.02°. In some embodiments, the salt has an X-ray powder diffraction pattern comprising peaks, in terms of 2θ, at about 17.14°, about 22.08°, and about 16.02°. In some embodiments, the salt has an X-ray powder diffraction pattern comprising peaks, in terms of 2θ, at about 17.14°, about 22.08°, about 16.02°, about 15.51°, about 11.05°, about 23.54°, and about 22.91°. In some embodiments, the salt has an X-ray powder diffraction pattern comprising peaks, in terms of 2θ, at about 17.14°, about 22.08°, about 16.02°, about 15.51°, about 11.05°, about 23.54°, about 22.91°, about 16.97°, about 24.70°, and about 25.51°. One aspect of the present invention is directed to a Compound 1 malonate salt having an X-ray powder diffraction pattern comprising one or more peaks listed in Table 18. In some embodiments, the salt has an X-ray powder diffraction pattern substantially as shown in FIG. 27, wherein by “substantially” is meant that the reported peaks can vary by about ±0.2 °2θ and also that the relative intensities of the reported peaks can vary.

In some embodiments, the Compound 1 malonate salt has a differential scanning calorimetry thermogram comprising an endotherm with an extrapolated onset temperature between about 130° C. and about 160° C. In some embodiments, the Compound 1 malonate salt has a differential scanning calorimetry thermogram comprising an endotherm with an extrapolated onset temperature at about 143° C. In some embodiments, the Compound 1 malonate salt has a differential scanning calorimetry thermogram comprising an endotherm with an associated heat flow of about 82 joules per gram. In some embodiments, the Compound 1 malonate salt has a thermogravimetric analysis profile substantially as shown in FIG. 28, wherein by “substantially” is meant that the reported TGA features can vary by about ±5° C. and by about ±2% weight change.

In some embodiments, the Compound 1 malonate salt has a differential scanning calorimetry thermogram substantially as shown in FIG. 28, wherein by “substantially” is meant that the reported DSC features can vary by about ±6° C. and by about ±20 joules per gram.

In some embodiments, the Compound 1 malonate salt has a dynamic moisture sorption profile substantially as shown in FIG. 29, wherein by “substantially” is meant that the reported DMS features can vary by about ±5% relative humidity and by about ±5% weight change.

Form I of Compound 1 malonate salt can be prepared by any of the suitable procedures known in the art for preparing crystalline polymorphs. In some embodiments Form I of Compound 1 malonate salt can be prepared as described in Example 8. In some embodiments, Form I of Compound 1 malonate salt can be prepared by slurrying crystalline Compound 1 malonate salt containing one or more crystalline forms other than Form I. In some embodiments, Form I of Compound 1 malonate salt can be prepared by recrystallizing crystalline Compound 1 malonate salt containing one or more crystalline forms other than Form I.

### Compound 1 Hemimalonate Salt.

One aspect of the present invention pertains to (R)-8-chloro-1-methyl-2,3,4,5-tetrahydro-1H-3-benzazepine hemimalonate salt, Form I (Compound 1 hemimalonate salt, Form I). The physical properties of Compound 1 hemimalonate salt, Form I are summarized in Table 19 below.

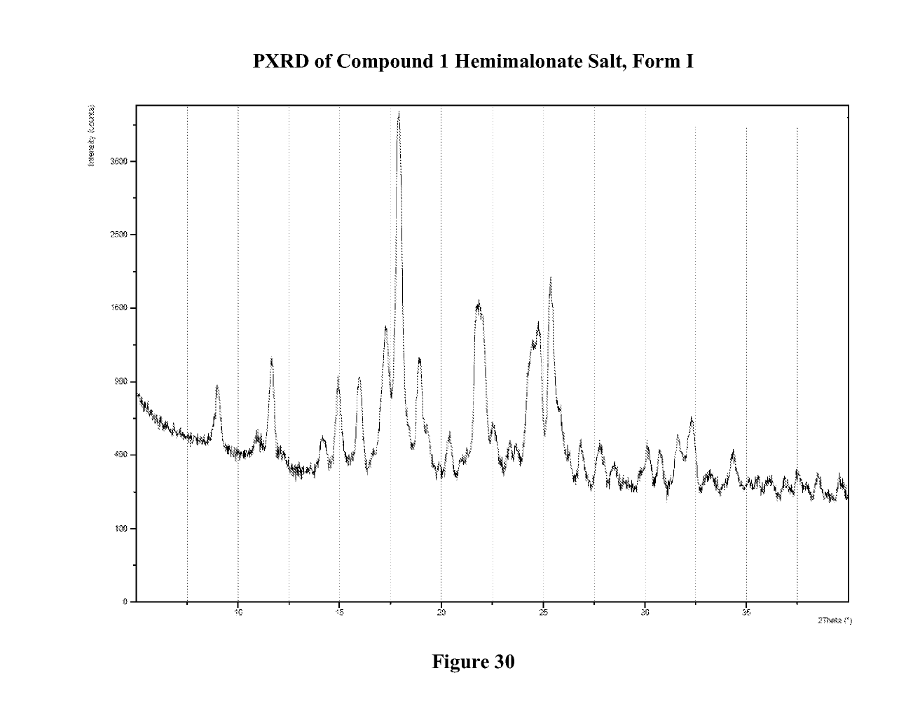

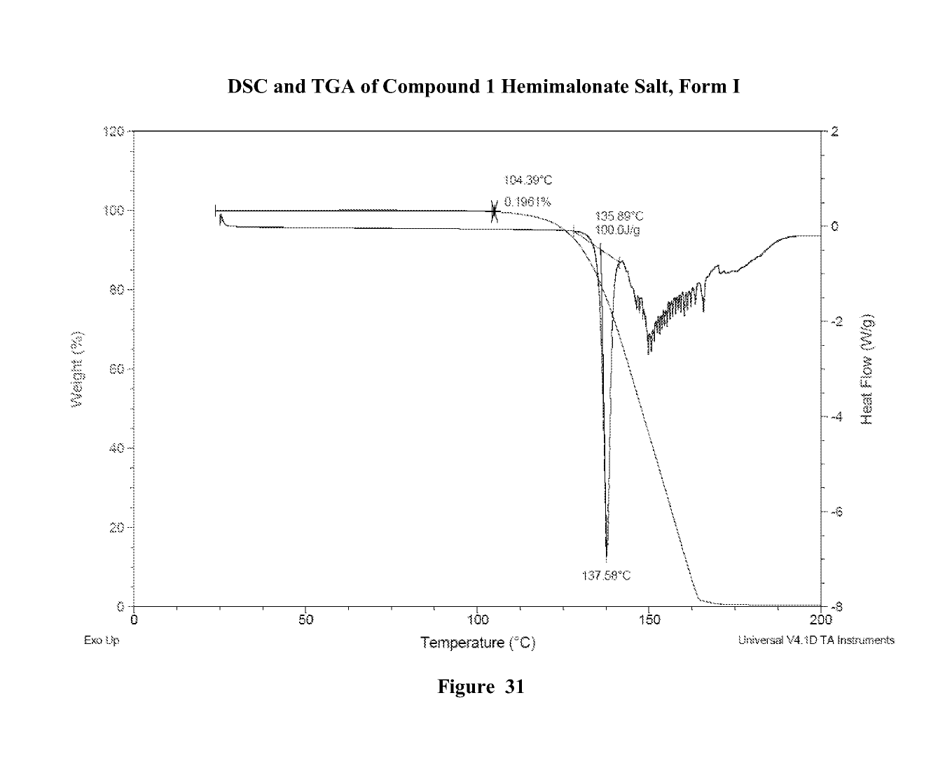

TABLE 19

| | Compound 1 Hemimalonate Salt, Form I |
|---|---|
| PXRD | FIG. 30: Peaks of ≧15% relative intensity at 11.66, 14.93, |
| | 15.98, 17.27, 17.90, 18.92, 21.81, 22.07, 24.25, 24.48, |
| | 24.77, and 25.37 °2θ |
| TGA | FIG. 31: <0.2% weight loss up to about 105° C. |
| DSC | FIG. 31: extrapolated onset temperature about 136° C.; |
| | enthalpy of fusion about 100 J/g |

Compound 1 hemimalonate salt, Form I had a melting onset at about 135-136° C. The TGA showed complete volatilization of the salt after melting.

Certain X-ray powder diffraction peaks for Form I of Compound 1 hemimalonate salt are shown in Table 20 below.

TABLE 20

| Pos. (°2θ) | Rel. Int. (%) |
|---|---|
| 9.00 | 10.58 |
| 10.94 | 3.33 |
| 11.66 | 18.12 |
| 14.17 | 5.34 |
| 14.93 | 15.65 |
| 15.98 | 16.07 |
| 17.27 | 27.09 |
| 17.90 | 100.00 |
| 18.92 | 19.50 |
| 19.29 | 7.29 |
| 20.39 | 5.42 |
| 21.81 | 32.41 |
| 22.07 | 27.90 |
| 22.54 | 7.42 |
| 23.36 | 5.00 |
| 23.70 | 4.40 |
| 24.25 | 17.52 |
| 24.48 | 23.66 |
| 24.77 | 27.91 |
| 25.37 | 41.49 |
| 25.86 | 10.53 |
| 26.27 | 3.71 |
| 26.83 | 5.20 |
| 27.82 | 4.75 |
| 28.48 | 2.86 |
| 30.15 | 5.00 |
| 30.74 | 4.93 |
| 31.65 | 7.02 |
| 32.29 | 9.54 |
| 33.18 | 2.09 |
| 34.32 | 5.08 |
| 35.57 | 2.27 |
| 36.12 | 1.87 |
| 36.90 | 1.81 |
| 37.51 | 2.50 |
| 37.96 | 1.47 |
| 38.51 | 2.79 |
| 39.56 | 2.57 |

One aspect of the present invention is directed to a Compound 1 hemimalonate salt having an X-ray powder diffraction pattern comprising a peak, in terms of 2θ, at about 17.90°. In some embodiments, the salt has an X-ray powder diffraction pattern comprising a peak, in terms of 2θ, at about 25.37°. In some embodiments, the salt has an X-ray powder diffraction pattern comprising peaks, in terms of 2θ, at about 17.90° and about 25.37°. In some embodiments, the salt has an X-ray powder diffraction pattern comprising peaks, in terms of 2θ, at about 17.90° and about 21.81°. In some embodiments, the salt has an X-ray powder diffraction pattern comprising peaks, in terms of 2θ, at about 17.90°, about 25.37°, and about 21.81°. In some embodiments, the salt has an X-ray powder diffraction pattern comprising peaks, in terms of 2θ, at about 17.90°, about 25.37°, about 21.81°, about 24.77°, about 22.07°, about 17.27°, and about 24.48°. In some embodiments, the salt has an X-ray powder diffraction pattern comprising peaks, in terms of 2θ, at about 17.90°, about 25.37°, about 21.81°, about 24.77°, about 22.07°, about 17.27°, about 24.48°, about 18.92°, about 11.66°, and about 24.25°. One aspect of the present invention is directed to a Compound 1 hemimalonate salt having an X-ray powder diffraction pattern comprising one or more peaks listed in Table 20. In some embodiments, the salt has an X-ray powder diffraction pattern substantially as shown in FIG. 30, wherein by “substantially” is meant that the reported peaks can vary by about ±0.2 °2θ and also that the relative intensities of the reported peaks can vary.

In some embodiments, the Compound 1 hemimalonate salt has a differential scanning calorimetry thermogram comprising an endotherm with an extrapolated onset temperature between about 120° C. and about 150° C. In some embodiments, the Compound 1 hemimalonate salt has a differential scanning calorimetry thermogram comprising an endotherm with an extrapolated onset temperature at about 136° C. In some embodiments, the Compound 1 hemimalonate salt has a differential scanning calorimetry thermogram comprising an endotherm with an associated heat flow of about 100 joules per gram. In some embodiments, the Compound 1 hemimalonate salt has a thermogravimetric analysis profile substantially as shown in FIG. 31, wherein by “substantially” is meant that the reported TGA features can vary by about ±5° C. and by about ±2% weight change.

In some embodiments, the Compound 1 hemimalonate salt has a differential scanning calorimetry thermogram substantially as shown in FIG. 31, wherein by “substantially” is meant that the reported DSC features can vary by about ±6° C. and by about ±20 joules per gram.

Form I of Compound 1 hemimalonate salt can be prepared by any of the suitable procedures known in the art for preparing crystalline polymorphs. In some embodiments Form I of Compound 1 hemimalonate salt can be prepared as described in Example 9. In some embodiments, Form I of Compound 1 hemimalonate salt can be prepared by slurrying crystalline Compound 1 hemimalonate salt containing one or more crystalline forms other than Form I. In some embodiments, Form I of Compound 1 hemimalonate salt can be prepared by recrystallizing crystalline Compound 1 hemimalonate salt containing one or more crystalline forms other than Form I.

### Compound 1 Glycolate Salt

One aspect of the present invention pertains to a crystalline form of (R)-8-chloro-1-methyl-2,3,4,5-tetrahydro-1H-3-benzazepine glycolate salt (Compound 1 glycolate salt). In some embodiments, the crystalline form of (R)-8-chloro-1-methyl-2,3,4,5-tetrahydro-1H-3-benzazepine glycolate salt is Form I (Compound 1 glycolate salt, Form I). The physical properties of Form I of Compound 1 glycolate salt are summarized in Table 21 below.

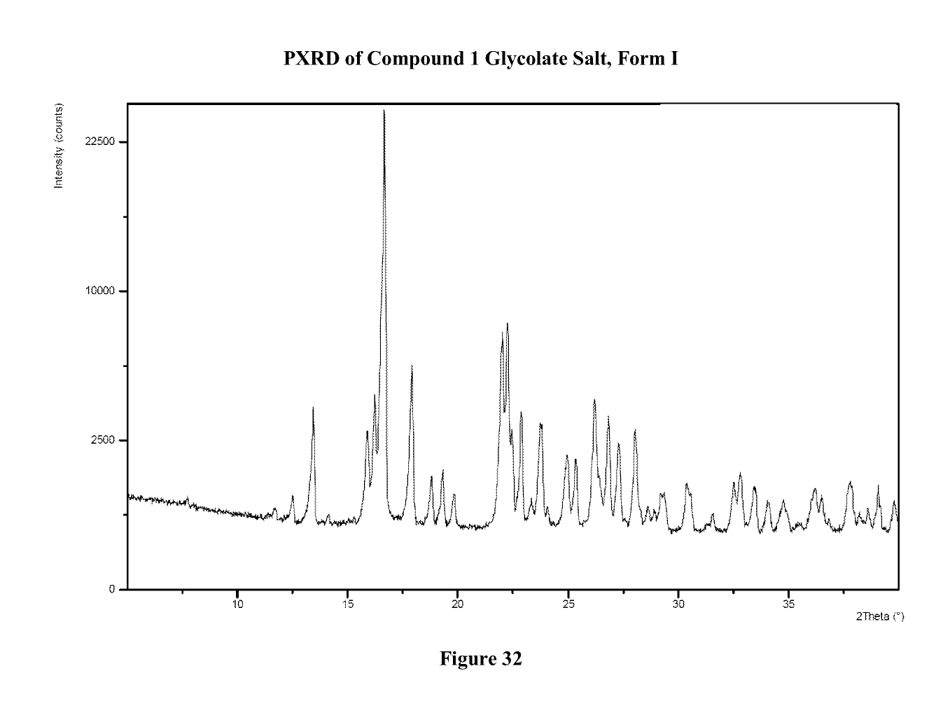

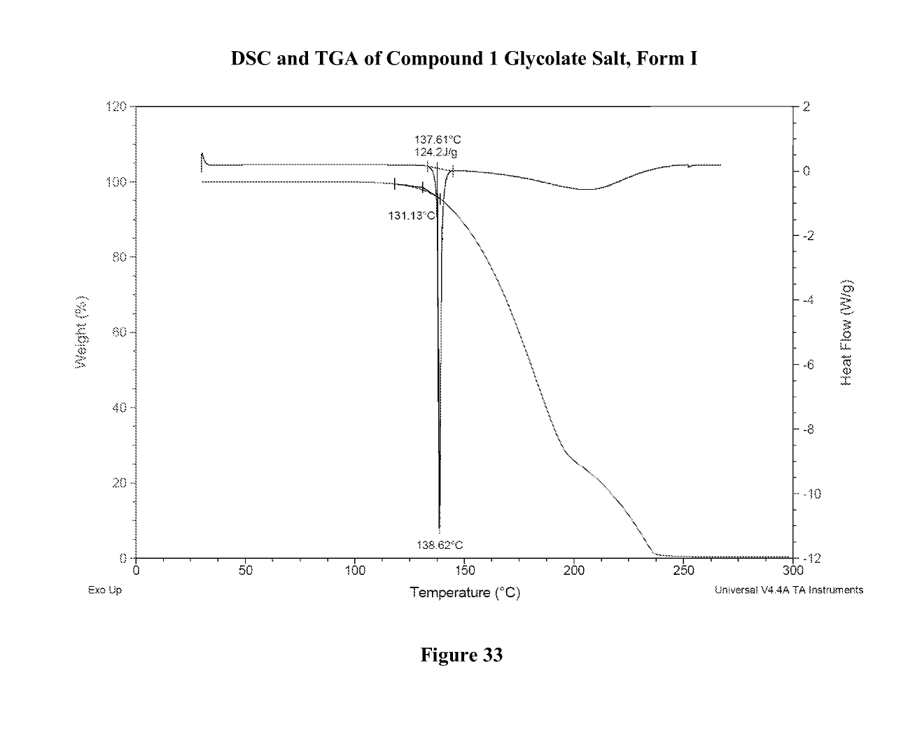

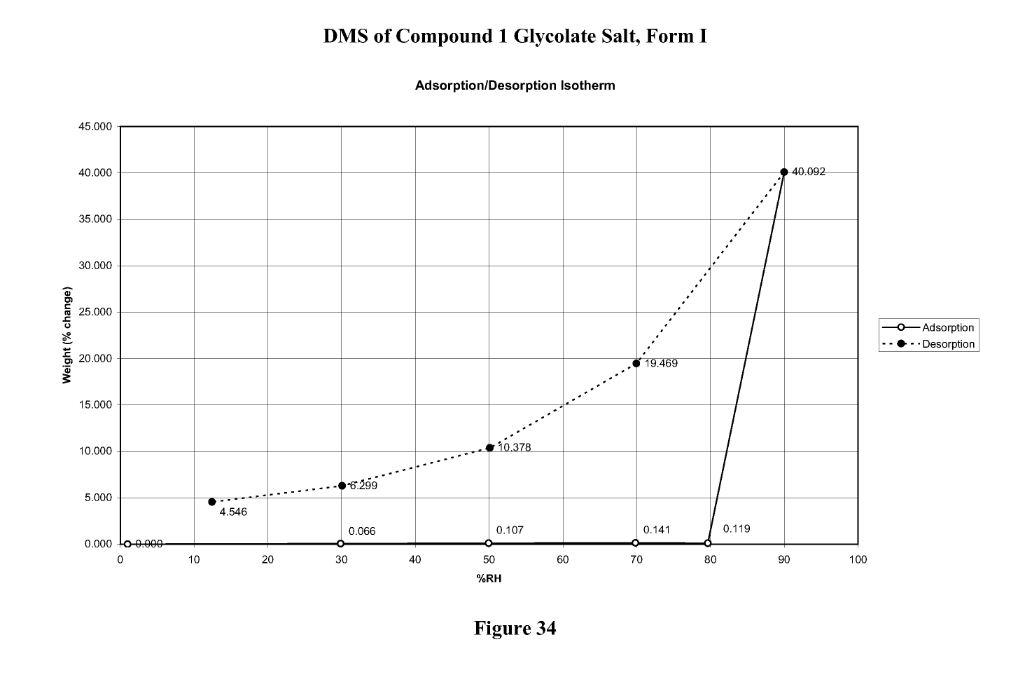

TABLE 21

| | Compound 1 Glycolate Salt, Form I |
|---|---|
| PXRD | FIG. 32: Peaks of ≧10% relative intensity at 13.45, 16.24, |
| | 16.67, 17.92, 22.01, 22.25, 22.88, 23.75, 23.82, 26.20, and |
| | 26.83 °2θ, |
| TGA | FIG. 33: negligible weight loss up to about 120° C. |
| DSC | FIG. 33: extrapolated onset temperature about 138° C.; |
| | enthalpy of fusion 124 J/g |
| DMS | FIG. 34: ~40% weight gain at about 90% RH |

Compound 1 glycolate salt, Form I was an anhydrous crystalline material with a melting onset of ˜138° C. It was non-solvated salt by TGA. During DMS analysis Compound 1 glycolate salt, Form I was deliquescent between 80 and 90% RH.

Certain X-ray powder diffraction peaks for Form I of Compound 1 glycolate salt are shown in Table 22 below.

TABLE 22

| Pos. (°2θ) | Rel. Int. (%) |
|---|---|
| 11.71 | 0.68 |
| 12.52 | 1.73 |
| 13.45 | 12.53 |
| 14.12 | 0.42 |
| 15.90 | 9.32 |
| 16.24 | 14.96 |
| 16.67 | 100.00 |
| 17.92 | 20.59 |
| 18.81 | 3.97 |
| 19.32 | 4.48 |
| 19.83 | 2.33 |
| 22.01 | 26.13 |
| 22.25 | 29.97 |
| 22.45 | 9.65 |
| 22.88 | 12.28 |
| 23.34 | 1.84 |
| 23.75 | 10.57 |
| 23.82 | 10.37 |
| 24.96 | 6.33 |
| 25.35 | 5.86 |
| 26.20 | 14.15 |
| 26.83 | 11.82 |
| 27.30 | 7.72 |
| 28.05 | 9.35 |
| 28.62 | 1.40 |
| 29.24 | 2.25 |
| 29.34 | 2.38 |
| 30.38 | 3.25 |
| 30.57 | 2.26 |
| 31.56 | 0.86 |
| 32.51 | 3.44 |
| 32.82 | 4.16 |
| 33.45 | 3.04 |
| 34.07 | 1.70 |
| 34.76 | 1.82 |
| 36.21 | 2.92 |
| 36.52 | 2.03 |
| 37.80 | 3.41 |
| 38.61 | 1.14 |
| 39.07 | 3.00 |

One aspect of the present invention is directed to a crystalline form of Compound 1 glycolate salt having an X-ray powder diffraction pattern comprising a peak, in terms of 2θ, at about 16.67°. In some embodiments, the crystalline form has an X-ray powder diffraction pattern comprising a peak, in terms of 2θ, at about 22.25°. In some embodiments, the crystalline form has an X-ray powder diffraction pattern comprising peaks, in terms of 2θ, at about 16.67° and about 22.25°. In some embodiments, the crystalline form has an X-ray powder diffraction pattern comprising peaks, in terms of 2θ, at about 16.67° and about 22.01°. In some embodiments, the crystalline form has an X-ray powder diffraction pattern comprising peaks, in terms of 2θ, at about 16.67°, about 22.25°, and about 22.01°. In some embodiments, the crystalline form has an X-ray powder diffraction pattern comprising peaks, in terms of 2θ, at about 16.67°, about 22.25°, about 22.01°, about 17.92°, about 16.24°, about 26.20°, and about 13.45°. In some embodiments, the crystalline form has an X-ray powder diffraction pattern comprising peaks, in terms of 2θ, at about 16.67°, about 22.25°, about 22.01°, about 17.92°, about 16.24°, about 26.20°, about 13.45°, about 22.88°, about 23.75°, and about 26.83°. One aspect of the present invention is directed to a crystalline form of Compound 1 glycolate salt having an X-ray powder diffraction pattern comprising one or more peaks listed in Table 22. In some embodiments, the crystalline form has an X-ray powder diffraction pattern substantially as shown in FIG. 32, wherein by “substantially” is meant that the reported peaks can vary by about ±0.2 °2θ and also that the relative intensities of the reported peaks can vary.

In some embodiments, the crystalline form of Compound 1 glycolate salt has a differential scanning calorimetry thermogram comprising an endotherm with an extrapolated onset temperature between about 120° C. and about 150° C. In some embodiments, the crystalline form of Compound 1 glycolate salt has a differential scanning calorimetry thermogram comprising an endotherm with an extrapolated onset temperature at about 138° C. In some embodiments, the crystalline form of Compound 1 glycolate salt has a differential scanning calorimetry thermogram comprising an endotherm with an associated heat flow of about 124 joules per gram. In some embodiments, the crystalline form of Compound 1 glycolate salt has a thermogravimetric analysis profile substantially as shown in FIG. 33, wherein by “substantially” is meant that the reported TGA features can vary by about ±5° C. and by about ±2% weight change.

In some embodiments, the crystalline form of Compound 1 glycolate salt has a differential scanning calorimetry thermogram substantially as shown in FIG. 33, wherein by “substantially” is meant that the reported DSC features can vary by about ±6° C. and by about ±20 joules per gram.

In some embodiments, the crystalline form of Compound 1 glycolate salt has a dynamic moisture sorption profile substantially as shown in FIG. 34, wherein by “substantially” is meant that the reported DMS features can vary by about ±5% relative humidity and by about ±5% weight change.

Form I of Compound 1 glycolate salt can be prepared by any of the suitable procedures known in the art for preparing crystalline polymorphs. In some embodiments Form I of Compound 1 glycolate salt can be prepared as described in Example 10. In some embodiments, Form I of Compound 1 glycolate salt can be prepared by slurrying crystalline Compound 1 glycolate salt containing one or more crystalline forms other than Form I. In some embodiments, the crystalline form of Compound 1 glycolate salt can be prepared by recrystallizing crystalline Compound 1 glycolate salt containing one or more crystalline forms other than Form I.

One aspect of the present invention pertains to processes for preparing a pharmaceutical composition comprising admixing a crystalline salt of the present invention, and a pharmaceutically acceptable carrier.

One aspect of the present invention pertains to processes for preparing a bulk pharmaceutical composition comprising admixing a crystalline salt of the present invention, and a pharmaceutically acceptable carrier.

One aspect of the present invention pertains to methods for weight management, comprising administering to an individual in need thereof, a therapeutically effective amount of a crystalline salt of the present invention.

One aspect of the present invention pertains to the use of crystalline salts of the present invention, in the manufacture of a medicament for weight management in an individual.

One aspect of the present invention pertains to crystalline salts of the present invention, for use in a method of treatment of the human or animal body by therapy.

One aspect of the present invention pertains to crystalline salts of the present invention, for use in a method of weight management.

One aspect of the present invention pertains to crystalline salts of the present invention, for use in a method of weight loss.

One aspect of the present invention pertains to crystalline salts of the present invention, for use in a method of maintenance of weight loss.

One aspect of the present invention pertains to crystalline salts of the present invention, for use in a method of decreasing food consumption.

One aspect of the present invention pertains to crystalline salts of the present invention, for use in a method of increasing meal-related satiety.

One aspect of the present invention pertains to crystalline salts of the present invention, for use in a method of reducing pre-meal hunger.

One aspect of the present invention pertains to crystalline salts of the present invention, for use in a method of reducing intra-meal food intake.

One aspect of the present invention pertains to crystalline salts of the present invention, for use in a method of weight management further comprising a reduced-calorie diet.

One aspect of the present invention pertains to crystalline salts of the present invention, for use in a method of weight management further comprising a program of regular exercise.

One aspect of the present invention pertains to crystalline salts of the present invention, for use in a method of weight management further comprising a reduced-calorie diet and a program of regular exercise.

One aspect of the present invention pertains to crystalline salts of the present invention, for use in a method of weight management in an obese patient with an initial body mass index ≧30 kg/m².

One aspect of the present invention pertains to crystalline salts of the present invention, for use in a method of weight management in an overweight patient with an initial body mass index ≧27 kg/m² in the presence of at least one weight related co-morbid condition.

One aspect of the present invention pertains to crystalline salts of the present invention, for use in a method of weight management in an overweight patient with an initial body mass index ≧27 kg/m² in the presence of at least one weight related co-morbid condition selected from: hypertension, dyslipidemia, cardiovascular disease, glucose intolerance, and sleep apnea.

One aspect of the present invention pertains to crystalline salts of the present invention, for use in a method of weight management in an individual with an initial body mass index ≧30 kg/m².

One aspect of the present invention pertains to crystalline salts of the present invention, for use in a method of weight management in an individual with an initial body mass index ≧27 kg/m².

One aspect of the present invention pertains to crystalline salts of the present invention, for use in a method of weight management in an individual with an initial body mass index ≧27 kg/m² in the presence of at least one weight related co-morbid condition.

One aspect of the present invention pertains to crystalline salts of the present invention, for use in a method of weight management in an individual with an initial body mass index ≧27 kg/m² in the presence of at least one weight related co-morbid condition selected from: hypertension, dyslipidemia, cardiovascular disease, glucose intolerance, and sleep apnea.

One aspect of the present invention pertains to crystalline salts of the present invention, for use in a method of weight management in an individual with an initial body mass index ≧25 kg/m².

One aspect of the present invention pertains to crystalline salts of the present invention, for use in a method of weight management in an individual with an initial body mass index ≧25 kg/m² in the presence of at least one weight related co-morbid condition.

One aspect of the present invention pertains to crystalline salts of the present invention, for use in a method of weight management in an individual with an initial body mass index ≧25 kg/m² in the presence of at least one weight related co-morbid condition selected from: hypertension, dyslipidemia, cardiovascular disease, glucose intolerance, and sleep apnea.

One aspect of the present invention pertains to crystalline salts of the present invention, for use in a method of weight management in combination with phentermine.

### Hydrates and Solvates

It is understood that when the phrase “pharmaceutically acceptable salts, solvates, and hydrates” or the phrase “pharmaceutically acceptable salt, solvate, or hydrate” is used when referring to compounds described herein, it embraces pharmaceutically acceptable solvates and/or hydrates of the compounds, pharmaceutically acceptable salts of the compounds, as well as pharmaceutically acceptable solvates and/or hydrates of pharmaceutically acceptable salts of the compounds. It is also understood that when the phrase “pharmaceutically acceptable solvates and hydrates” or the phrase “pharmaceutically acceptable solvate or hydrate” is used when referring to compounds described herein that are salts, it embraces pharmaceutically acceptable solvates and/or hydrates of such salts.

It will be apparent to those skilled in the art that the dosage forms described herein may comprise, as the active component, either a salts or crystalline form thereof as described herein, or a solvate or hydrate thereof. Moreover, various hydrates and solvates of the salts or crystalline form thereof described herein will find use as intermediates in the manufacture of pharmaceutical compositions. Typical procedures for making and identifying suitable hydrates and solvates, outside those mentioned herein, are well known to those in the art; see for example, pages 202-209 of K.J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999.

Accordingly, one aspect of the present invention pertains to methods of administering hydrates and solvates of salts or crystalline forms thereof described herein and/or their pharmaceutically acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, Del.), Avantium Technologies (Amsterdam) and Aptuit (Greenwich, Conn.).

Isotopes

The present disclosure includes all isotopes of atoms occurring in the present salts and crystalline forms thereof. Isotopes include those atoms having the same atomic number but different mass numbers. One aspect of the present invention includes every combination of one or more atoms in the present salts and crystalline forms thereof that is replaced with an atom having the same atomic number but a different mass number. One such example is the replacement of an atom that is the most naturally abundant isotope, such as ¹H or ¹²C, found in one the present salts and crystalline forms thereof, with a different atom that is not the most naturally abundant isotope, such as ²H or ³H (replacing ¹H), or ¹¹C, ¹³C, or ¹⁴C (replacing ¹²C). A salt wherein such a replacement has taken place is commonly referred to as being isotopically-labeled. Isotopic-labeling of the present salts and crystalline forms thereof can be accomplished using any one of a variety of different synthetic methods know to those of ordinary skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸C. An isotope of fluorine includes ¹⁸F. An isotope of sulfur includes ³⁵S. An isotope of chlorine includes ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I, and ¹³¹I. Another aspect of the present invention includes compositions, such as, those prepared during synthesis, preformulation, and the like, and pharmaceutical compositions, such as, those prepared with the intent of using in a mammal for the treatment of one or more of the disorders described herein, comprising one or more of the present salts and crystalline forms thereof, wherein the naturally occurring distribution of the isotopes in the composition is perturbed. Another aspect of the present invention includes compositions and pharmaceutical compositions comprising salts and crystalline forms thereof as described herein wherein the salt is enriched at one or more positions with an isotope other than the most naturally abundant isotope. Methods are readily available to measure such isotope perturbations or enrichments, such as, mass spectrometry, and for isotopes that are radio-isotopes additional methods are available, such as, radio-detectors used in connection with HPLC or GC.

### Pharmaceutical Compositions

A further aspect of the present invention pertains to pharmaceutical compositions comprising one or more salts according to any of the salt embodiments disclosed herein and one or more pharmaceutically acceptable carriers. Some embodiments pertain to pharmaceutical compositions comprising a salt according to any of the salt embodiments disclosed herein and a pharmaceutically acceptable carrier. Some embodiments pertain to pharmaceutical compositions comprising any subcombination of salts according to any of the salt embodiments disclosed herein.

Another aspect of the present invention pertains to methods of producing pharmaceutical compositions comprising admixing one or more salts according to any of the salt embodiments disclosed herein and one or more pharmaceutically acceptable carriers. Some embodiments pertain to a method of producing a pharmaceutical composition comprising admixing a salt according to any of the salt embodiments disclosed herein and a pharmaceutically acceptable carrier. Some embodiments pertain to a methods of producing pharmaceutical compositions comprising admixing any subcombination of salts according to any of the salt embodiments disclosed herein and a pharmaceutically acceptable carrier.

Rapidly disintegrating or dissolving dosage forms are useful for the rapid absorption, particularly buccal absorption, of pharmaceutically active agents. Fast-dissolve dosage forms are beneficial to gastric by-pass patients, pediatrics, geriatrics and patients with dysphagia, who have difficulty in swallowing typical solid dosage forms, such as caplets and tablets. Fast-dissolve dosage forms also improve compliance with dosing regimens in patients with high average daily pill burdens such as obese patients in whom hypertension, atherosclerosis, diabetes, and certain types of cancer, are commonplace.

Additionally, fast-dissolve dosage forms circumvent drawbacks associated with, for example, chewable dosage forms, wherein the length of time an active agent remains in a patient's mouth plays an important role in determining the amount of taste masking and the extent to which a patient may object to throat grittiness of the active agent.

To overcome such problems manufacturers have developed a number of fast-dissolve solid dose oral formulations. These are available from manufacturers including Cima Labs, Fuisz Technologies Ltd., Prographarm, R. P. Scherer, Yamanouchi-Shaklee, and McNeil-PPC, Inc. All of these manufacturers market different types of rapidly dissolving solid oral dosage forms.

Cima Labs markets OraSolv®, which is an effervescent direct compression tablet having an oral dissolution time of five to thirty seconds, and DuraSolv®, which is a direct compression tablet having a taste-masked active agent and an oral dissolution time of 15 to 45 seconds. Cima's U.S. Pat. No. 5,607,697, for “Taste Masking Microparticles for Oral Dosage Forms,” describes a solid dosage form consisting of coated microparticles that disintegrate in the mouth. The microparticle core of Cima's patented oral dosage form has a pharmaceutical agent and one or more sweet-tasting compounds having a negative heat of solution wherein the sweet-tasting compound can be mannitol, sorbitol, a mixture of an artificial sweetener and menthol, a mixture of sugar and menthol, or methyl salicylate. The microparticle core is coated, at least partially, with a material that retards dissolution in the mouth and masks the taste of the pharmaceutical agent. The microparticles are then compressed to form a tablet. Cima's patent discloses that other excipients can also be added to the tablet formulation.

WO 98/46215 for “Rapidly Dissolving Robust Dosage Form,” assigned to Cima Labs, is directed to a hard, compressed, fast-dissolve formulation having an active ingredient and a matrix of at least a non-direct compression filler and lubricant. A non-direct compression filler is typically not free-flowing, in contrast to a direct compression (DC grade) filler, and usually requires additionally processing to form free-flowing granules.

Cima also has U.S. patents and international patent applications directed to effervescent dosage forms (U.S. Pat. Nos. 5,503,846, 5,223,264, and 5,178,878) and tableting aids for rapidly dissolving dosage forms (U.S. Pat. Nos. 5,401,513 and 5,219,574), and rapidly dissolving dosage forms for water soluble drugs (WO 98/14179 for “Taste-Masked Microcapsule Composition and Methods of Manufacture”).

Fuisz Technologies, now part of BioVail, markets Flash Dose®, which is a direct compression tablet containing a processed excipient called Shearform®. Shearform® is a cotton candy-like substance of mixed polysaccharides converted to amorphous fibers. U.S. patents describing this technology include U.S. Pat. No. 5,871,781 for “Apparatus for Making Rapidly Dissolving Dosage Units;” U.S. Pat. No. 5,869,098 for “Fast-Dissolving Comestible Units Formed Under High-Speed/High-Pressure Conditions;” U.S. Pat. Nos. 5,866,163, 5,851,553, and 5,622,719, all for “Process and Apparatus for Making Rapidly Dissolving Dosage Units and Product Therefrom;” U.S. Pat. No. 5,567,439 for “Delivery of Controlled-Release Systems;” and U.S. Pat. No. 5,587,172 for “Process for Forming Quickly Dispersing Comestible Unit and Product Therefrom.”

Prographarm markets Flashtab®, which is a fast-dissolve tablet having a disintegrating agent such as carboxymethyl cellulose, a swelling agent such as a modified starch, and a taste-masked active agent. The tablets have an oral disintegration time of under one minute (U.S. Pat. No. 5,464,632).

R. P. Scherer markets Zydis®, which is a freeze-dried tablet having an oral dissolution time of 2 to 5 seconds. Lyophilized tablets are costly to manufacture and difficult to package because of the tablets sensitivity to moisture and temperature. U.S. Pat. No. 4,642,903 (R. P. Scherer Corp.) refers to a fast-dissolve dosage formulation prepared by dispersing a gas throughout a solution or suspension to be freeze-dried. U.S. Pat. No. 5,188,825 (R. P. Scherer Corp.) refers to freeze-dried dosage forms prepared by bonding or complexing a water-soluble active agent to or with an ion exchange resin to form a substantially water insoluble complex, which is then mixed with an appropriate carrier and freeze dried. U.S. Pat. No. 5,631,023 (R. P. Scherer Corp.) refers to freeze-dried drug dosage forms made by adding xanthan gum to a suspension of gelatin and active agent. Finally, U.S. Pat. No. 5,827,541 (R. P. Scherer Corp.) discloses a process for preparing solid pharmaceutical dosage forms of hydrophobic substances. The process involves freeze-drying a dispersion containing a hydrophobic active ingredient and a surfactant, in a non-aqueous phase; and a carrier material, in an aqueous phase.

Yamanouchi-Shaklee markets Wowtab®, which is a tablet having a combination of a low moldability and a high moldability saccharide. U.S. patents covering this technology include U.S. Pat. No. 5,576,014 for “Intrabuccally Dissolving Compressed Moldings and Production Process Thereof,” and U.S. Pat. No. 5,446,464 for “Intrabuccally Disintegrating Preparation and Production Thereof.”

Other companies owning rapidly dissolving technology include Janssen Pharmaceutica. U.S. patents assigned to Janssen describe rapidly dissolving tablets having two polypeptide (or gelatin) components and a bulking agent, wherein the two components have a net charge of the same sign, and the first component is more soluble in aqueous solution than the second component. See U.S. Pat. No. 5,807,576 for “Rapidly Dissolving Tablet;” U.S. Pat. No. 5,635,210 for “Method of Making a Rapidly Dissolving Tablet;” U.S. Pat. No. 5,595,761 for “Particulate Support Matrix for Making a Rapidly Dissolving Tablet;” U.S. Pat. No. 5,587,180 for “Process for Making a Particulate Support Matrix for Making a Rapidly Dissolving Tablet;” and U.S. Pat. No. 5,776,491 for “Rapidly Dissolving Dosage Form.”

Eurand America, Inc. has U.S. patents directed to a rapidly dissolving effervescent composition having a mixture of sodium bicarbonate, citric acid, and ethyl cellulose (U.S. Pat. Nos. 5,639,475 and 5,709,886).

L.A.B. Pharmaceutical Research owns U.S. patents directed to effervescent-based rapidly dissolving formulations having a pharmaceutically active ingredient and an effervescent couple comprising an effervescent acid and an effervescent base (U.S. Pat. Nos. 5,807,578 and 5,807,577).

Schering Corporation has technology relating to buckle tablets having an active agent, an excipient (which can be a surfactant) or at least one of sucrose, lactose, or sorbitol, and either magnesium stearate or sodium dodecyl sulfate (U.S. Pat. Nos. 5,112,616 and 5,073,374).

Laboratoire L. LaFon owns technology directed to conventional dosage forms made by lyophilization of an oil-in-water emulsion in which at least one of the two phases contains a surfactant (U.S. Pat. No. 4,616,047). For this type of formulation, the active ingredient is maintained in a frozen suspension state and is tableted without micronization or compression, as such processes could damage the active agent.

Takeda Chemicals Inc., Ltd. owns technology directed to a method of making a fast dissolving tablet in which an active agent and a moistened, soluble carbohydrate are compression molded into a tablet, followed by drying of the tablets (U.S. Pat. No. 5,501,861).

Finally, Elan's U.S. Pat. No. 6,316,029, for “Rapidly Disintegrating Oral Dosage Form,” disclosed fast-dissolve dosage forms comprising nanoparticulate active agents.

Fast-dissolve tablets as described in the prior art are generally characterized as having short disintegration times when exposed, for example, to the aqueous environment of a patient's mouth. These short disintegration times can be achieved through careful adjustment of a tablet formulation and through the use of active pharmaceutical ingredients with high aqueous solubility. The new salts of Compound 1 described herein are all highly water-soluble and therefore they can be used to prepare fast-dissolve dosage forms, which are useful for, inter alia, weight management.

Salts of the present invention or a solvate, hydrate or physiologically functional derivative thereof can be used as active ingredients in pharmaceutical compositions, specifically as 5-HT_{2C}-receptor modulators. The term “active ingredient” as defined in the context of a “pharmaceutical composition” and is intended to mean a component of a pharmaceutical composition that provides the primary pharmacological effect, as opposed to an “inactive ingredient” which would generally be recognized as providing no pharmaceutical benefit.

The dose when using the salts of the present invention can vary within wide limits and as is customary and is known to the physician, it is to be tailored to the individual conditions in each individual case. It depends, for example, on the nature and severity of the illness to be treated, on the condition of the patient, on the salt employed or on whether an acute or chronic disease state is treated or prophylaxis conducted or on whether further active compounds are administered in addition to the salts of the present invention. Representative doses of the present invention include, but are not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular salt employed, whether a drug delivery system is utilized, on whether an acute or chronic disease state is being treated or prophylaxis conducted or on whether further active compounds are administered in addition to the salts of the present invention and as part of a drug combination. The dosage regimen for treating a disease condition with the salts and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

Some embodiments of the present invention include a method of producing a pharmaceutical composition for “combination-therapy” comprising admixing at least one salt according to any of the salt embodiments disclosed herein, together with at least one known pharmaceutical agent as described herein and a pharmaceutically acceptable carrier.

It is noted that when the salts of the present invention are utilized as active ingredients in a pharmaceutical composition, these are not intended for use only in humans, but in other non-human mammals as well. Indeed, recent advances in the area of animal health-care mandate that consideration be given for the use of active agents, such as 5-HT_{2C}-receptor modulators, for the treatment of a 5-HT_{2C}-receptor-associated diseases or disorders in companionship animals (e.g., cats, dogs, etc.) and in livestock animals (e.g., cows, chickens, fish, etc.). Those of ordinary skill in the art are readily credited with understanding the utility of such salts in such settings.

One aspect of the present invention pertains to methods for weight management, comprising administering to an individual in need thereof, a therapeutically effective amount of a pharmaceutical composition of the present invention.

One aspect of the present invention pertains to pharmaceutical compositions of the present invention, for use in a method of treatment of the human or animal body by therapy.

One aspect of the present invention pertains to pharmaceutical compositions of the present invention, for use in a method of weight management.

One aspect of the present invention pertains to pharmaceutical compositions of the present invention, for use in a method of weight loss.

One aspect of the present invention pertains to pharmaceutical compositions of the present invention, for use in a method of maintenance of weight loss.

One aspect of the present invention pertains to pharmaceutical compositions of the present invention, for use in a method of decreasing food consumption.

One aspect of the present invention pertains to pharmaceutical compositions of the present invention, for use in a method of increasing meal-related satiety.

One aspect of the present invention pertains to pharmaceutical compositions of the present invention, for use in a method of reducing pre-meal hunger.

One aspect of the present invention pertains to pharmaceutical compositions of the present invention, for use in a method of reducing intra-meal food intake.

One aspect of the present invention pertains to pharmaceutical compositions of the present invention, for use in a method of weight management further comprising a reduced-calorie diet.

One aspect of the present invention pertains to pharmaceutical compositions of the present invention, for use in a method of weight management further comprising a program of regular exercise.

One aspect of the present invention pertains to pharmaceutical compositions of the present invention, for use in a method of weight management further comprising a reduced-calorie diet and a program of regular exercise.

One aspect of the present invention pertains to pharmaceutical compositions of the present invention, for use in a method of weight management in an obese patient with an initial body mass index ≧30 kg/m².

One aspect of the present invention pertains to pharmaceutical compositions of the present invention, for use in a method of weight management in an overweight patient with an initial body mass index ≧27 kg/m² in the presence of at least one weight related co-morbid condition.

One aspect of the present invention pertains to pharmaceutical compositions of the present invention, for use in a method of weight management in an overweight patient with an initial body mass index ≧27 kg/m² in the presence of at least one weight related co-morbid condition selected from: hypertension, dyslipidemia, cardiovascular disease, glucose intolerance, and sleep apnea.

One aspect of the present invention pertains to pharmaceutical compositions of the present invention, for use in a method of weight management in an individual with an initial body mass index ≧30 kg/m².

One aspect of the present invention pertains to pharmaceutical compositions of the present invention, for use in a method of weight management in an individual with an initial body mass index ≧27 kg/m².

One aspect of the present invention pertains to pharmaceutical compositions of the present invention, for use in a method of weight management in an individual with an initial body mass index ≧27 kg/m² in the presence of at least one weight related co-morbid condition.

One aspect of the present invention pertains to pharmaceutical compositions of the present invention, for use in a method of weight management in an individual with an initial body mass index ≧27 kg/m² in the presence of at least one weight related co-morbid condition selected from: hypertension, dyslipidemia, cardiovascular disease, glucose intolerance, and sleep apnea.

One aspect of the present invention pertains to pharmaceutical compositions of the present invention, for use in a method of weight management in an individual with an initial body mass index ≧25 kg/m².

One aspect of the present invention pertains to pharmaceutical compositions of the present invention, for use in a method of weight management in an individual with an initial body mass index ≧25 kg/m² in the presence of at least one weight related co-morbid condition.

One aspect of the present invention pertains to pharmaceutical compositions of the present invention, for use in a method of weight management in an individual with an initial body mass index ≧25 kg/m² in the presence of at least one weight related co-morbid condition selected from: hypertension, dyslipidemia, cardiovascular disease, glucose intolerance, and sleep apnea.

One aspect of the present invention pertains to pharmaceutical compositions of the present invention, for use in a method of weight management in combination with phentermine.

One aspect of the present invention pertains to dosage forms comprising a therapeutically effective amount of a salt selected from: a pharmaceutically acceptable salt of (R)-8-chloro-1-methyl-2,3,4,5-tetrahydro-1H-3-benzazepine and pharmaceutically acceptable solvates and hydrates thereof, wherein the dosage form is a fast-dissolve dosage form.

In some embodiments, the salt has an aqueous solubility of at least about 400 mg/mL at about room temperature.

In some embodiments, the salt has an aqueous solubility of at least about 500 mg/mL at about room temperature.

In some embodiments, the salt has an aqueous solubility of at least about 600 mg/mL at about room temperature.

In some embodiments, the salt has an aqueous solubility of at least about 700 mg/mL at about room temperature.

In some embodiments, the salt has an aqueous solubility of at least about 800 mg/mL at about room temperature.

In some embodiments, the salt has an aqueous solubility of at least about 900 mg/mL at about room temperature.

In some embodiments, the salt has an aqueous solubility of at least about 1000 mg/mL at about room temperature.

In some embodiments, the salt has an aqueous solubility of: at least about 400 mg/mL at about room temperature; at least about 500 mg/mL at about room temperature; at least about 600 mg/mL at about room temperature; at least about 700 mg/mL at about room temperature; at least about 800 mg/mL at about room temperature; at least about 900 mg/mL at about room temperature; or at least about 1000 mg/mL at about room temperature.

In some embodiments, the salt has an aqueous solubility of between about 400 mg/mL at about room temperature and about 2000 mg/mL at about room temperature.

In some embodiments, the salt has an aqueous solubility of between about 400 mg/mL at about room temperature and about 1000 mg/mL at about room temperature.

In some embodiments, the salt has an aqueous solubility of between about 400 mg/mL at about room temperature and about 900 mg/mL at about room temperature.

In some embodiments, the salt has an aqueous solubility of between about 400 mg/mL at about room temperature and about 800 mg/mL at about room temperature.

In some embodiments, the salt has an aqueous solubility of between about 400 mg/mL at about room temperature and about 700 mg/mL at about room temperature.

In some embodiments, the salt has an aqueous solubility of between about 400 mg/mL at about room temperature and about 600 mg/mL at about room temperature.

In some embodiments, the salt has an aqueous solubility of between about 400 mg/mL at about room temperature and about 500 mg/mL at about room temperature.

In some embodiments, the salt has an aqueous solubility of between about 500 mg/mL at about room temperature and about 2000 mg/mL at about room temperature.

In some embodiments, the salt has an aqueous solubility of between about 500 mg/mL at about room temperature and about 1000 mg/mL at about room temperature.

In some embodiments, the salt has an aqueous solubility of between about 500 mg/mL at about room temperature and about 900 mg/mL at about room temperature.

In some embodiments, the salt has an aqueous solubility of between about 500 mg/mL at about room temperature and about 800 mg/mL at about room temperature.

In some embodiments, the salt has an aqueous solubility of between about 500 mg/mL at about room temperature and about 700 mg/mL at about room temperature.

In some embodiments, the salt has an aqueous solubility of between about 500 mg/mL at about room temperature and about 600 mg/mL at about room temperature.

In some embodiments, the salt has an aqueous solubility of between about 600 mg/mL at about room temperature and about 2000 mg/mL at about room temperature.

In some embodiments, the salt has an aqueous solubility of between about 600 mg/mL at about room temperature and about 1000 mg/mL at about room temperature.

In some embodiments, the salt has an aqueous solubility of between about 600 mg/mL at about room temperature and about 900 mg/mL at about room temperature.

In some embodiments, the salt has an aqueous solubility of between about 600 mg/mL at about room temperature and about 800 mg/mL at about room temperature.

In some embodiments, the salt has an aqueous solubility of between about 600 mg/mL at about room temperature and about 700 mg/mL at about room temperature.

In some embodiments, the salt has an aqueous solubility of between about 700 mg/mL at about room temperature and about 2000 mg/mL at about room temperature.

In some embodiments, the salt has an aqueous solubility of between about 700 mg/mL at about room temperature and about 1000 mg/mL at about room temperature.

In some embodiments, the salt has an aqueous solubility of between about 700 mg/mL at about room temperature and about 900 mg/mL at about room temperature.

In some embodiments, the salt has an aqueous solubility of between about 700 mg/mL at about room temperature and about 800 mg/mL at about room temperature.

In some embodiments, the salt has an aqueous solubility of between about 800 mg/mL at about room temperature and about 2000 mg/mL at about room temperature.

In some embodiments, the salt has an aqueous solubility of between about 800 mg/mL at about room temperature and about 1000 mg/mL at about room temperature.

In some embodiments, the salt has an aqueous solubility of between about 800 mg/mL at about room temperature and about 900 mg/mL at about room temperature.

In some embodiments, the salt has an aqueous solubility of between about 900 mg/mL at about room temperature and about 2000 mg/mL at about room temperature.

In some embodiments, the salt has an aqueous solubility of between about 900 mg/mL at about room temperature and about 1000 mg/mL at about room temperature.

In some embodiments, the salt has an aqueous solubility of between about 1000 mg/mL at about room temperature and about 2000 mg/mL at about room temperature.

In some embodiments, the salt is selected from: (R)-8-chloro-1-methyl-2,3,4,5-tetrahydro-1H-3-benzazepine hydrochloride, or a solvate or hydrate thereof.

In some embodiments, the salt is selected from: (R)-8-chloro-1-methyl-2,3,4,5-tetrahydro-1H-3-benzazepine hydrochloride or a solvate or hydrate thereof.

In some embodiments, the dosage form comprises (R)-8-chloro-1-methyl-2,3,4,5-tetrahydro-1H-3-benzazepine hydrochloride hemihydrate.

In some embodiments, the dosage form comprises (R)-8-chloro-1-methyl-2,3,4,5-tetrahydro-1H-3-benzazepine hydrochloride hemihydrate, Form III.

One aspect of the present invention pertains to dosage forms comprising a therapeutically effective amount of a salt of the present invention.

In some embodiments, the dosage form comprises a therapeutically effective amount of (R)-8-chloro-1-methyl-2,3,4,5-tetrahydro-1H-3-benzazepine bisulfate salt.

In some embodiments, the dosage form comprises a therapeutically effective amount of (R)-8-chloro-1-methyl-2,3,4,5-tetrahydro-1H-3-benzazepine hemisulfate salt hydrate.

In some embodiments, the dosage form comprises a therapeutically effective amount of (R)-8-chloro-1-methyl-2,3,4,5-tetrahydro-1H-3-benzazepine mesylate salt.

In some embodiments, the dosage form comprises a therapeutically effective amount of (R)-8-chloro-1-methyl-2,3,4,5-tetrahydro-1H-3-benzazepine hydrobromide salt hemihydrate.

In some embodiments, the dosage form comprises a therapeutically effective amount of (R)-8-chloro-1-methyl-2,3,4,5-tetrahydro-1H-3-benzazepine nitrate salt.

In some embodiments, the dosage form comprises a therapeutically effective amount of (R)-8-chloro-1-methyl-2,3,4,5-tetrahydro-1H-3-benzazepine sesqui-oxalate salt-cocrystal.

In some embodiments, the dosage form comprises a therapeutically effective amount of (R)-8-chloro-1-methyl-2,3,4,5-tetrahydro-1H-3-benzazepine adipate salt.

In some embodiments, the dosage form comprises a therapeutically effective amount of (R)-8-chloro-1-methyl-2,3,4,5-tetrahydro-1H-3-benzazepine malonate salt.

In some embodiments, the dosage form comprises a therapeutically effective amount of (R)-8-chloro-1-methyl-2,3,4,5-tetrahydro-1H-3-benzazepine hemimalonate salt.

In some embodiments, the dosage form further comprises one or more pharmaceutically acceptable excipients.

One aspect of the present invention pertains to dosage forms for oral administration to an individual in need of weight management.

In some embodiments, the weight management comprises weight loss.

In some embodiments, the weight management comprises maintenance of weight loss.

In some embodiments, the weight management comprises decreased food consumption.

In some embodiments, the weight management comprises increasing meal-related satiety.

In some embodiments, the weight management comprises reducing pre-meal hunger.

In some embodiments, the weight management comprises reducing intra-meal food intake.

In some embodiments, the weight management further comprises a reduced-calorie diet.

In some embodiments, the weight management further comprises a program of regular exercise.

In some embodiments, the weight management further comprises both a reduced-calorie diet and a program of regular exercise.

In some embodiments, the individual in need of weight management is an obese patient with an initial body mass index ≧30 kg/m².

In some embodiments, the individual in need of weight management is an overweight patient with an initial body mass index ≧27 kg/m² in the presence of at least one weight related comorbid condition.

In some embodiments, the weight related co-morbid condition is selected from: hypertension, dyslipidemia, cardiovascular disease, glucose intolerance, and sleep apnea.

In some embodiments, the dosage form is for administration in combination with phentermine.

### Indications

Obesity is a life-threatening disorder in which there is an increased risk of morbidity and mortality arising from concomitant diseases such as, but not limited to, type II diabetes, hypertension, stroke, certain forms of cancers and gallbladder disease.

Obesity has become a major healthcare issue in the Western World and increasingly in some third world countries. The increase in the number of obese people is due largely to the increasing preference for high fat content foods but also, and this can be a more important factor, the decrease in activity in most people's lives. In spite of the growing awareness of the health concerns linked to obesity the percentage of individuals that are overweight or obese continues to increase. The most significant concern, from a public health perspective, is that children who are overweight grow up to be overweight or obese adults, and accordingly are at greater risk for major health problems. Therefore, it appears that the number of individuals that are overweight or obese will continue to increase.

Whether someone is classified as overweight or obese is generally determined on the basis of his or her body mass index (BMI) which is calculated by dividing body weight (kg) by height squared (m²). Thus, the units for BMI are kg/m². BMI is more highly correlated with body fat than any other indicator of height and weight. A person is considered overweight when they have a BMI in the range of 25-30 kg/m², whereas a person with a BMI over 30 kg/m² is classified as obese. Obesity is further divided into three classes: Class I (BMI of about 30 to about 34.9 kg/m²), Class II (BMI of about 35 to 39.9 kg/m²) and Class III (about 40 kg/m² or greater); see Table below for complete classifications.

Classification Of Weight By Body Mass Index (BMI)

| BMI | CLASSIFICATION |
|---|---|
| <18.5 | Underweight |
| 18.5-24.9 | Normal |
| 25.0-29.9 | Overweight |
| 30.0-34.9 | Obesity (Class I) |
| 35.0-39.9 | Obesity (Class II) |
| >40 | Extreme Obesity (Class III) |

As the BMI increases for an individual there is an increased risk of morbidity and mortality relative to an individual with normal BMI. Accordingly, overweight and obese individuals (BMI of about 25 kg/m² and above) are at increased risk for physical ailments such as, but not limited to, high blood pressure, cardiovascular disease (particularly hypertension), high blood cholesterol, dyslipidemia, type II (non-insulin dependent) diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), diseases of reproduction (such as sexual dysfunction, both male and female, including male erectile dysfunction), bladder control problems (such as stress incontinence), uric acid nephrolithiasis, psychological disorders (such as depression, eating disorders, distorted body image, and low self esteem). Research has shown that even a modest reduction in body weight can correspond to a significant reduction in the risk of developing other ailments, such as, but not limited to, coronary heart disease.

As mentioned above, obesity increases the risk of developing cardiovascular diseases. Coronary insufficiency, atheromatous disease, and cardiac insufficiency are at the forefront of the cardiovascular complications induced by obesity. The incidence of coronary diseases is doubled in subjects less than 50 years of age who are 30% overweight. The diabetes patient faces a 30% reduced lifespan. After age 45, people with diabetes are about three times more likely than people without diabetes to have significant heart disease and up to five times more likely to have a stroke. These findings emphasize the inter-relations between risks factors for type 2 diabetes and coronary heart disease and the potential value of an integrated approach to the prevention of these conditions based on the prevention of obesity [Perry, I. J., et al. BMJ 310, 560-564 (1995)]. It is estimated that if the entire population had an ideal weight, the risk of coronary insufficiency would decrease by 25% and the risk of cardiac insufficiency and of cerebral vascular accidents by 35%.

Diabetes has also been implicated in the development of kidney disease, eye diseases and nervous-system problems. Kidney disease, also called nephropathy, occurs when the kidney's “filter mechanism” is damaged and protein leaks into urine in excessive amounts and eventually the kidney fails. Diabetes is also a leading cause of damage to the retina and increases the risk of cataracts and glaucoma. Finally, diabetes is associated with nerve damage, especially in the legs and feet, which interferes with the ability to sense pain and contributes to serious infections. Taken together, diabetes complications are one of the nation's leading causes of death.

The first line of treatment for individuals that are overweight or obese is to offer diet and life style advice, such as, reducing the fat content of their diet and increasing their physical activity. However many patients find these difficult to maintain and need additional help from drug therapy to sustain results from these efforts.

Most currently marketed products have been unsuccessful as treatments for obesity owing to a lack of efficacy or unacceptable side-effect profiles. The most successful drug so far was the indirectly acting 5-hydroxytryptamine (5-HT) agonist d-fenfluramine (Redux™) but reports of cardiac valve defects in up to one third of the patient population led to its withdrawal by the FDA in 1998.

The 5-HT_{2C} receptor is recognized as a well-accepted receptor target for the treatment of obesity, psychiatric, and other disorders. See, for example, Halford et al., Serotonergic Drugs Effects on Appetite Expression and Use for the Treatment of Obesity, Drugs 2007; 67 (1): 27-55; Naughton et al., A Review Of The Role Of Serotonin Receptors In Psychiatric Disorders. Human Psychopharmacology (2000), 15(6), 397-415.

(R)-8-Chloro-1-methyl-2,3,4,5-tetrahydro-1H-3-benzazepine hydrochloride (lorcaserin hydrochloride) is an agonist of the 5-HT_{2C} receptor and shows effectiveness at reducing obesity in animal models and humans. In a phase 3 human clinical trial evaluating the safety and efficacy of lorcaserin for weight management, statistical significance (p<0.0001) was achieved on all three of the hierarchically ordered co-primary endpoints for patients treated with lorcaserin versus placebo. Treatment with lorcaserin was generally very well tolerated. An assessment of echocardiograms indicated no apparent drug-related effect on the development of US Food and Drug Administration (FDA)-defined valvulopathy over the two-year treatment period. The hierarchically ordered endpoints were the proportion of patients achieving 5% or greater weight loss after 12 months, the difference in mean weight loss compared to placebo after 12 months, and the proportion of patients achieving 10% or greater weight loss after 12 months. Compared to placebo, using an intent-to-treat last observation carried forward (ITT-LOCF) analysis, treatment with lorcaserin was associated with highly statistically significant (p <0.0001) categorical and average weight loss from baseline after 12 months: 47.5% of lorcaserin patients lost greater than or equal to 5% of their body weight from baseline compared to 20.3% in the placebo group. This result satisfied the efficacy benchmark in the most recent FDA draft guidance. Average weight loss of 5.8% of body weight, or 12.7 pounds, was achieved in the lorcaserin group, compared to 2.2% of body weight, or 4.7 pounds, in the placebo group. Statistical separation from placebo was observed by Week 2, the first post-baseline measurement. 22.6% of lorcaserin patients lost greater than or equal to 10% of their body weight from baseline, compared to 7.7% in the placebo group. Lorcaserin patients who completed 52 weeks of treatment according to the protocol lost an average of 8.2% of body weight, or 17.9 pounds, compared to 3.4%, or 7.3 pounds, in the placebo group (p<0.0001).

In addition, the 5-HT_{2C} receptor is also involved in other diseases, conditions and disorders, such as, obsessive compulsive disorder, some forms of depression, and epilepsy. Accordingly, 5-HT_{2C} receptor agonists can have anti-panic properties, and properties useful for the treatment of sexual dysfunction. In addition, 5-HT_{2C} receptor agonists are useful for the treatment of psychiatric symptoms and behaviors in individuals with eating disorders such as, but not limited to, anorexia nervosa and bulimia nervosa. Individuals with anorexia nervosa often demonstrate social isolation. Anorexic individuals often present symptoms of being depressed, anxious, obsession, perfectionistic traits, and rigid cognitive styles as well as sexual disinterest. Other eating disorders include, anorexia nervosa, bulimia nervosa, binge eating disorder (compulsive eating) and ED-NOS (i.e., eating disorders not otherwise specified—an official diagnosis). An individual diagnosed with ED-NOS possess atypical eating disorders including situations in which the individual meets all but a few of the criteria for a particular diagnosis. What the individual is doing with regard to food and weight is neither normal nor healthy.

The 5-HT_{2C} receptor plays a role in Alzheimer Disease (AD). Therapeutic agents currently prescribed for Alzheimer's disease (AD) are cholinomimetic agents that act by inhibiting the enzyme acetylcholinesterase. The resulting effect is increased levels of acetylcholine, which modestly improves neuronal function and cognition in patients with AD. Although, dysfunction of cholinergic brain neurons is an early manifestation of AD, attempts to slow the progression of the disease with these agents have had only modest success, perhaps because the doses that can be administered are limited by peripheral cholinergic side effects, such as tremors, nausea, vomiting, and dry mouth. In addition, as AD progresses, these agents tend to lose their effectiveness due to continued cholinergic neuronal loss.

Therefore, there is a need for agents that have beneficial effects in AD, particularly in alleviating symptoms by improving cognition and slowing or inhibiting disease progression, without the side effects observed with current therapies. Therefore, serotonin 5-HT_{2C} receptors, which are exclusively expressed in brain, are attractive targets.

Another disease, disorder or condition that can is associated with the function of the 5-HT_{2C} receptor is erectile dysfunction (ED). Erectile dysfunction is the inability to achieve or maintain an erection sufficiently rigid for intercourse, ejaculation, or both. An estimated 20-30 million men in the United States have this condition at some time in their lives. The prevalence of the condition increases with age. Five percent of men 40 years of age report ED. This rate increases to between 15% and 25% by the age of 65, and to 55% in men over the age of 75 years.

Erectile dysfunction can result from a number of distinct problems. These include loss of desire or libido, the inability to maintain an erection, premature ejaculation, lack of emission, and inability to achieve an orgasm. Frequently, more than one of these problems presents themselves simultaneously. The conditions may be secondary to other disease states (typically chronic conditions), the result of specific disorders of the urogenital system or endocrine system, secondary to treatment with pharmacological agents (e.g. antihypertensive drugs, antidepressant drugs, antipsychotic drugs, etc.) or the result of psychiatric problems. Erectile dysfunction, when organic, is primarily due to vascular irregularities associated with atherosclerosis, diabetes, and hypertension.

There is evidence for use of a serotonin 5-HT_{2C} agonist for the treatment of sexual dysfunction in males and females. The serotonin 5-HT_{2C} receptor is involved with the processing and integration of sensory information, regulation of central monoaminergic systems, and modulation of neuroendocrine responses, anxiety, feeding behavior, and cerebrospinal fluid production [Tecott, L. H., et al. Nature 374: 542-546 (1995)]. In addition, the serotonin 5-HT_{2C} receptor has been implicated in the mediation of penile erections in rats, monkeys, and humans.

In summary, the 5-HT_{2C} receptor is a validated and well-accepted receptor target for the prophylaxis and/or treatment of 5-HT_{2C} mediated receptor diseases and disorders, such as, obesity, eating disorders, psychiatric disorders, Alzheimer Disease, sexual dysfunction and disorders related thereto. It can be seen that there exists a need for selective 5-HT_{2C} receptor agonists that can safely address these needs. The present invention is directed to these, as well as other, important ends.

One aspect of the present invention pertains to methods for weight management, comprising administering to an individual in need thereof, a therapeutically effective amount of a salt, a pharmaceutical composition, or a dosage form of the present invention.

In some embodiments, the weight management comprises weight loss.

In some embodiments, the weight management comprises maintenance of weight loss.

In some embodiments, the weight management comprises decreased food consumption.

In some embodiments, the weight management comprises increasing meal-related satiety.

In some embodiments, the weight management comprises reducing pre-meal hunger.

In some embodiments, the weight management comprises reducing intra-meal food intake.

In some embodiments, the weight management further comprises a reduced-calorie diet.

In some embodiments, the weight management further comprises a program of regular exercise.

In some embodiments, the weight management further comprises both a reduced-calorie diet and a program of regular exercise.

In some embodiments, the individual in need of weight management is an obese patient with an initial body mass index ≧30 kg/m².

In some embodiments, the individual in need of weight management is an overweight patient with an initial body mass index ≧27 kg/m² in the presence of at least one weight related comorbid condition.

In some embodiments, the individual in need of weight management is an overweight patient with an initial body mass index ≧27 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧30 kg/m².

In some embodiments, the individual in need of weight management has an initial body mass index ≧27 kg/m².

In some embodiments, the individual in need of weight management has an initial body mass index ≧27 kg/m² in the presence of at least one weight related comorbid condition.

In some embodiments, the individual in need of weight management has an initial body mass index ≧27 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧25 kg/m².

In some embodiments, the individual in need of weight management has an initial body mass index ≧25 kg/m² in the presence of at least one weight related comorbid condition.

In some embodiments, the individual in need of weight management has an initial body mass index ≧25 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 20 kg/m² in the presence of at least one weight related comorbid condition.

In some embodiments, the individual in need of weight management has an initial body mass index ≧about 20 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 21 kg/m² in the presence of at least one weight related comorbid condition.

In some embodiments, the individual in need of weight management has an initial body mass index ≧about 21 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 22 kg/m² in the presence of at least one weight related comorbid condition.

In some embodiments, the individual in need of weight management has an initial body mass index ≧about 22 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 23 kg/m² in the presence of at least one weight related comorbid condition.

In some embodiments, the individual in need of weight management has an initial body mass index ≧about 23 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 24 kg/m² in the presence of at least one weight related comorbid condition.

In some embodiments, the individual in need of weight management has an initial body mass index ≧about 24 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 25 kg/m² in the presence of at least one weight related comorbid condition.

In some embodiments, the individual in need of weight management has an initial body mass index ≧about 25 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 26 kg/m² in the presence of at least one weight related comorbid condition.

In some embodiments, the individual in need of weight management has an initial body mass index ≧about 26 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 27 kg/m² in the presence of at least one weight related comorbid condition.

In some embodiments, the individual in need of weight management has an initial body mass index ≧about 27 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 28 kg/m² in the presence of at least one weight related comorbid condition.

In some embodiments, the individual in need of weight management has an initial body mass index ≧about 28 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 29 kg/m² in the presence of at least one weight related comorbid condition.

In some embodiments, the individual in need of weight management has an initial body mass index ≧about 29 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 30 kg/m² in the presence of at least one weight related comorbid condition.

In some embodiments, the individual in need of weight management has an initial body mass index ≧about 30 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 31 kg/m² in the presence of at least one weight related comorbid condition.

In some embodiments, the individual in need of weight management has an initial body mass index ≧about 31 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 32 kg/m² in the presence of at least one weight related comorbid condition.

In some embodiments, the individual in need of weight management has an initial body mass index ≧about 32 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 33 kg/m² in the presence of at least one weight related comorbid condition.

In some embodiments, the individual in need of weight management has an initial body mass index ≧about 33 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 34 kg/m² in the presence of at least one weight related comorbid condition.

In some embodiments, the individual in need of weight management has an initial body mass index ≧about 34 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 35 kg/m² in the presence of at least one weight related comorbid condition.

In some embodiments, the individual in need of weight management has an initial body mass index ≧about 35 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 36 kg/m² in the presence of at least one weight related comorbid condition.

In some embodiments, the individual in need of weight management has an initial body mass index ≧about 36 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 37 kg/m² in the presence of at least one weight related comorbid condition.

In some embodiments, the individual in need of weight management has an initial body mass index ≧about 37 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 38 kg/m² in the presence of at least one weight related comorbid condition.

In some embodiments, the individual in need of weight management has an initial body mass index ≧about 38 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 39 kg/m² in the presence of at least one weight related comorbid condition.

In some embodiments, the individual in need of weight management has an initial body mass index ≧about 39 kg/m² in the presence of at least one weight related comorbid condition selected from: hypertension, dyslipidemia, cardiovascular disease, glucose intolerance, and sleep apnea.

In some embodiments, the individual in need of weight management has an initial body mass index ≧about 40 kg/m² in the presence of at least one weight related comorbid condition.

In some embodiments, the individual in need of weight management has an initial body mass index ≧about 40 kg/m² in the presence of at least one weight related comorbid condition selected from: hypertension, dyslipidemia, cardiovascular disease, glucose intolerance, and sleep apnea.

In some embodiments, the method for weight management further comprises administering phentermine to the individual.

One aspect of the present invention pertains to methods for the treatment of a disorder related to 5-HT_{2C} receptor activity in an individual, comprising administering to an individual in need thereof, a therapeutically effective amount of a salt, a pharmaceutical composition, or a dosage form of the present invention.

One aspect of the present invention pertains to methods for the treatment of obesity, comprising administering to an individual in need thereof, a therapeutically effective amount of a salt, a pharmaceutical composition, or a dosage form of the present invention.

In some embodiments, the method for the treatment of obesity further comprises the administration or prescription of phentermine.

In some embodiments, the method for the treatment of obesity further comprises gastric electrical stimulation.

One aspect of the present invention pertains to methods for inducing weight loss, BMI loss, waist circumference loss or body fat percentage loss, comprising administering to an individual in need thereof, a therapeutically effective amount of a salt, a pharmaceutical composition, or a dosage form of the present invention.

One aspect of the present invention pertains to methods for inducing weight loss, BMI loss, waist circumference loss or body fat percentage loss in an individual in preparation of the individual for bariatric surgery, comprising administering to an individual in need thereof, a therapeutically effective amount of a salt, a pharmaceutical composition, or a dosage form of the present invention.

One aspect of the present invention pertains to methods for maintaining weight loss, BMI loss, waist circumference loss or body fat percentage loss in an individual, comprising administering to an individual in need thereof, a therapeutically effective amount of a salt, a pharmaceutical composition, or a dosage form of the present invention.

One aspect of the present invention pertains to methods for maintaining weight loss, BMI loss, waist circumference loss or body fat percentage loss in an individual following bariatric surgery, comprising administering to an individual in need thereof, a therapeutically effective amount of a salt, a pharmaceutical composition, or a dosage form of the present invention.

One aspect of the present invention pertains to methods for inducing satiety in an individual, comprising administering to an individual in need thereof, a therapeutically effective amount of a salt, a pharmaceutical composition, or a dosage form of the present invention.

One aspect of the present invention pertains to methods for decreasing food intake in an individual, comprising administering to an individual in need thereof, a therapeutically effective amount of a salt, a pharmaceutical composition, or a dosage form of the present invention.

One aspect of the present invention pertains to methods for decreasing hunger in an individual, comprising administering to an individual in need thereof, a therapeutically effective amount of a salt, a pharmaceutical composition, or a dosage form of the present invention.

One aspect of the present invention pertains to methods for decreasing food cravings in an individual, comprising administering to an individual in need thereof, a therapeutically effective amount of a salt, a pharmaceutical composition, or a dosage form of the present invention.

One aspect of the present invention pertains to methods for increasing intermeal interval in an individual, comprising administering to an individual in need thereof, a therapeutically effective amount of a salt, a pharmaceutical composition, or a dosage form of the present invention.

One aspect of the present invention pertains to methods for the treatment of a disorder selected from: schizophrenia, anxiety, depression, psychoses and alcohol addiction, comprising administering to an individual in need thereof, a therapeutically effective amount of a salt, a pharmaceutical composition, or a dosage form of the present invention.

In some embodiments, the disorder is schizophrenia.

In some embodiments, the disorder is anxiety.

In some embodiments, the disorder is depression.

In some embodiments, the disorder is psychoses.

In some embodiments, the disorder is alcohol addiction.

One aspect of the present invention pertains to the use of salts of the present invention, in the manufacture of a medicament for weight management in an individual.

In some embodiments, the weight management comprises weight loss.

In some embodiments, the weight management comprises maintenance of weight loss.

In some embodiments, the weight management comprises decreased food consumption.

In some embodiments, the weight management comprises increasing meal-related satiety.

In some embodiments, the weight management comprises reducing pre-meal hunger.

In some embodiments, the weight management comprises reducing intra-meal food intake.

In some embodiments, the weight management further comprises a reduced-calorie diet.

In some embodiments, the weight management further comprises a program of regular exercise.

In some embodiments, the weight management further comprises both a reduced-calorie diet and a program of regular exercise.

In some embodiments, the individual is an obese patient with an initial body mass index ≧30 kg/m².

In some embodiments, the individual is an overweight patient with an initial body mass index ≧27 kg/m² in the presence of at least one weight related comorbid condition.

In some embodiments, the individual is an overweight patient with an initial body mass index ≧27 kg/m² in the presence of at least one weight related comorbid condition selected from: hypertension, dyslipidemia, cardiovascular disease, glucose intolerance, and sleep apnea.

In some embodiments, the individual has an initial body mass index ≧30 kg/m².

In some embodiments, the individual has an initial body mass index ≧27 kg/m².

In some embodiments, the individual has an initial body mass index ≧27 kg/m² in the presence of at least one weight related comorbid condition.

In some embodiments, the individual has an initial body mass index ≧27 kg/m² in the presence of at least one weight related comorbid condition selected from: hypertension, dyslipidemia, cardiovascular disease, glucose intolerance, and sleep apnea.

In some embodiments, the individual has an initial body mass index ≧25 kg/m².

In some embodiments, the individual has an initial body mass index ≧25 kg/m² in the presence of at least one weight related comorbid condition.

In some embodiments, the individual has an initial body mass index ≧25 kg/m² in the presence of at least one weight related comorbid condition selected from: hypertension, dyslipidemia, cardiovascular disease, glucose intolerance, and sleep apnea.

In some embodiments, the medicament for weight management is used in combination with phentermine.

One aspect of the present invention pertains to the use of salts of the present invention, in the manufacture of a medicament for a disorder related to 5-HT_{2C} receptor activity in an individual.

One aspect of the present invention pertains to the use of salts of the present invention, in the manufacture of a medicament for the treatment of obesity in an individual.

In some embodiments, the treatment of obesity further comprises the administration or prescription of phentermine.

In some embodiments, the treatment of obesity further comprises gastric electrical stimulation.

One aspect of the present invention pertains to the use of salts of the present invention, in the manufacture of a medicament for inducing weight loss, BMI loss, waist circumference loss or body fat percentage loss in an individual.

One aspect of the present invention pertains to the use of salts of the present invention, in the manufacture of a medicament for inducing weight loss, BMI loss, waist circumference loss or body fat percentage loss in an individual in preparation of the individual for bariatric surgery.

One aspect of the present invention pertains to the use of salts of the present invention, in the manufacture of a medicament for maintaining weight loss, BMI loss, waist circumference loss or body fat percentage loss in an individual.

One aspect of the present invention pertains to the use of salts of the present invention, in the manufacture of a medicament for maintaining weight loss, BMI loss, waist circumference loss or body fat percentage loss in an individual following bariatric surgery.

One aspect of the present invention pertains to the use of salts of the present invention, in the manufacture of a medicament for inducing satiety in an individual.

One aspect of the present invention pertains to the use of salts of the present invention, in the manufacture of a medicament for decreasing food intake in an individual.

One aspect of the present invention pertains to the use of salts of the present invention, in the manufacture of a medicament for decreasing hunger in an individual.

One aspect of the present invention pertains to the use of salts of the present invention, in the manufacture of a medicament for decreasing food cravings in an individual.

One aspect of the present invention pertains to the use of salts of the present invention, in the manufacture of a medicament for increasing intermeal interval in an individual.

One aspect of the present invention pertains to the use of salts of the present invention, in the manufacture of a medicament for the treatment of a disorder selected from: schizophrenia, anxiety, depression, psychoses and alcohol addiction in an individual.

In some embodiments, the disorder is schizophrenia.

In some embodiments, the disorder is anxiety.

In some embodiments, the disorder is depression.

In some embodiments, the disorder is psychoses.

In some embodiments, the disorder is alcohol addiction.

One aspect of the present invention pertains to salts and pharmaceutical compositions of the present invention, for use in a method of treatment of a disorder related to 5-HT_{2C} receptor activity in an individual.

One aspect of the present invention pertains to salts and pharmaceutical compositions of the present invention, for use in a method of treatment of obesity in an individual.

In some embodiments, the method of treatment of obesity further comprises the administration or prescription of phentermine.

In some embodiments, the method of treatment of obesity further comprises gastric electrical stimulation.

One aspect of the present invention pertains to salts and pharmaceutical compositions of the present invention, for use in a method of inducing weight loss, BMI loss, waist circumference loss or body fat percentage loss in an individual.

One aspect of the present invention pertains to salts and pharmaceutical compositions of the present invention, for use in a method of inducing weight loss, BMI loss, waist circumference loss or body fat percentage loss in an individual in preparation of the individual for bariatric surgery.

One aspect of the present invention pertains to salts and pharmaceutical compositions of the present invention, for use in a method of maintaining weight loss, BMI loss, waist circumference loss or body fat percentage loss in an individual.

One aspect of the present invention pertains to salts and pharmaceutical compositions of the present invention, for use in a method of maintaining weight loss, BMI loss, waist circumference loss or body fat percentage loss in an individual following bariatric surgery.

One aspect of the present invention pertains to salts and pharmaceutical compositions of the present invention, for use in a method of inducing satiety in an individual.

One aspect of the present invention pertains to salts and pharmaceutical compositions of the present invention, for use in a method of decreasing food intake in an individual.

One aspect of the present invention pertains to salts and pharmaceutical compositions of the present invention, for use in a method of decreasing hunger in an individual.

One aspect of the present invention pertains to salts and pharmaceutical compositions of the present invention, for use in a method of decreasing food cravings in an individual.

One aspect of the present invention pertains to salts and pharmaceutical compositions of the present invention, for use in a method of increasing intermeal interval in an individual.

One aspect of the present invention pertains to salts and pharmaceutical compositions of the present invention, for use in a method of treatment of a disorder selected from: schizophrenia, anxiety, depression, psychoses and alcohol addiction in an individual.

In some embodiments, the disorder is schizophrenia.

In some embodiments, the disorder is anxiety.

In some embodiments, the disorder is depression.

In some embodiments, the disorder is psychoses.

In some embodiments, the disorder is alcohol addiction.

One aspect of the present invention pertains to methods for weight management, comprising administering to an individual in need thereof, a therapeutically effective amount of a salt, a pharmaceutical composition, or a dosage form of the present invention.

In some embodiments, the weight management comprises one or more of: weight loss, maintenance of weight loss, decreased food consumption, increasing meal-related satiety, reducing pre-meal hunger, and reducing intra-meal food intake.

In some embodiments, the weight management is as an adjunct to diet and exercise.

In some embodiments, the individual in need of weight management is selected from: an obese patient with an initial body mass index ≧30 kg/m²; an overweight patient with an initial body mass index ≧27 kg/m² in the presence of at least one weight related comorbid condition; an overweight patient with an initial body mass index ≧27 kg/m² in the presence of at least one weight related comorbid condition; wherein the weight related co-morbid condition is selected from: hypertension, dyslipidemia, cardiovascular disease, glucose intolerance, and sleep apnea.

In some embodiments, the method further comprises administering a second anti-obesity agent to the individual.

In some embodiments, the second anti-obesity agent is selected from: chlorphentermine, clortermine, phenpentermineu, and phentermine, and pharmaceutically acceptable salts, solvates, and hydrates thereof.

In some embodiments, the method further comprises administering an anti-diabetes agent to the individual.

In some embodiments, the anti-diabetes agent is metformin.

One aspect of the present invention pertains to uses of a salt of the present invention, in the manufacture of a medicament for weight management in an individual.

In some embodiments, the weight management comprises one or more of: weight loss, maintenance of weight loss, decreased food consumption, increasing meal-related satiety, reducing pre-meal hunger, and reducing intra-meal food intake.

In some embodiments, the medicament is used as an adjunct to diet and exercise.

In some embodiments, the individual in need of weight management is selected from: an obese patient with an initial body mass index ≧30 kg/m²; an overweight patient with an initial body mass index ≧27 kg/m² in the presence of at least one weight related comorbid condition; and an overweight patient with an initial body mass index ≧27 kg/m² in the presence of at least one weight related comorbid condition; wherein the weight related co-morbid condition is selected from: hypertension, dyslipidemia, cardiovascular disease, glucose intolerance, and sleep apnea.

In some embodiments, the medicament is used in combination with a second anti-obesity agent.

In some embodiments, the second anti-obesity agent is selected from: chlorphentermine, clortermine, phenpentermine, and phentermine, and pharmaceutically acceptable salts, solvates, and hydrates thereof.

In some embodiments, the medicament is used in combination with an anti-diabetes agent.

In some embodiments, the medicament is used in combination with an anti-diabetes agent; wherein the anti-diabetes agent is metformin.

One aspect of the present invention pertains to salts, pharmaceutical compositions, and dosage forms of the present invention, for use in a method of treatment of the human or animal body by therapy.

One aspect of the present invention pertains to salts, pharmaceutical compositions, and dosage forms of the present invention, for use in a method of weight management.

One aspect of the present invention pertains to salts, pharmaceutical compositions, and dosage forms, for use in a method of weight management; wherein the weight management comprises one or more of: weight loss, maintenance of weight loss, decreased food consumption, increasing meal-related satiety, reducing pre-meal hunger, and reducing intra-meal food intake.

One aspect of the present invention pertains to salts, pharmaceutical compositions, and dosage forms of the present invention, for use as an adjunct to diet and exercise for weight management.

One aspect of the present invention pertains to salts, pharmaceutical compositions, and dosage forms of the present invention, for use in a method of weight management; wherein the individual in need of weight management is selected from: an obese patient with an initial body mass index ≧30 kg/m²; an overweight patient with an initial body mass index ≧27 kg/m² in the presence of at least one weight related comorbid condition; and an overweight patient with an initial body mass index ≧27 kg/m² in the presence of at least one weight related comorbid condition; wherein the weight related co-morbid condition is selected from: hypertension, dyslipidemia, cardiovascular disease, glucose intolerance, and sleep apnea.

One aspect of the present invention pertains to salts, pharmaceutical compositions, and dosage forms of the present invention, for use in a method of weight management in combination with a second anti-obesity agent.

One aspect of the present invention pertains to salts, pharmaceutical compositions, and dosage forms of the present invention, for use in a method of weight management in combination with a second anti-obesity agent selected from: chlorphentermine, clortermine, phenpentermine, and phentermine, and pharmaceutically acceptable salts, solvates, and hydrates thereof.

One aspect of the present invention pertains to salts, pharmaceutical compositions, and dosage forms of the present invention, for use in a method of weight management in combination with an anti-diabetes agent; wherein the anti-diabetes agent is metformin.

### Combination Therapies

The salts of the present invention can be used in combination with suitable pharmaceutical agents.

In some embodiments the salts of the present invention can be used in combination with a second anti-obesity agent. Anti-obesity agents include, for example, adrenergic reuptake inhibitors, apolipoprotein-B secretion/microsomal triglyceride transfer protein inhibitors, β3 adrenergic receptor agonists, bombesin agonists, cannabinoid 1 receptor antagonists, cholescystokinin-A agonists, ciliary neutrotrophic factors, dopamine agonists, galanin antagonists, ghrelin receptor antagonists, glucagon-like peptide-1 receptor agonists, glucocorticoid receptor agonists or antagonists, histamine-3 receptor antagonists or reverse agonists, human agouti-related proteins, leptin receptor agonists, lipase inhibitors, MCR-4 agonists, melanin concentrating hormone antagonists, melanocyte-stimulating hormone receptor analogs, monoamine reuptake inhibitors, neuromedin U receptor agonists, neuropeptide-Y antagonists, orexin receptor antagonists, stimulants, sympathomimetic agents, thyromimetic agents, and urocortin binding protein antagonists.

In some embodiments, the second anti-obesity agent is selected from: 4-methylamphetamine, 5-HTP, amfecloral, amfepentorex, amfepramone, a minorex, amphetamine, amphetaminil, atomoxetine, benfluorex, benzphetamine, bromocriptine, bupropion, cathine, cathinone, cetilistat, chlorphentermine, ciclazindol, clobenzorex, cloforex, clominorex, clortermine, dapiclermin, dehydroepiandrosterone, dehydroepiandrosterone analogues, dexmethylphenidate, dextroamphetamine, dextromethamphetamine, difemetorex, dimethylcathinone, dinitrophenol, diphemethoxidine, ephedra, ephedrine, ethylamphetamine, etolorex, fenbutrazate, fencamfamine, fenethylline, fenproporex, fludorex, fluminorex, furfenorex, galactomannan, glucomannan, ibipinabant, indanorex, khat, L-dopa, leptin, a leptin analog, levopropylhexedrine, lisdexamfetamine, L-phenylalanine, L-tryptophan, L-tyrosine, N-[[trans-4-[(4,5-dihydro[l]benzothiepino[5,4-d]thiazol-2-yl)amino]cyclohexyl]methyl]methanesulfonamide, manifaxine, mazindol, mefenorex, metformin, methamphetamine, methylphenidate, naloxone, naltrexone, oleoyl-estrone, orlistat, otenabant, oxyntomodulin, P57, pemoline, peptide YY, phendimetrazine, phenethylamine, phenmetrazine, phenpentermine, phentermine, phenylpropanolamine, pipradrol, prolintane, propylhexedrine, pseudoephedrine, pyrovalerone, radafaxine, reboxetine, rimonabant, setazindol, sibutramine, simmondsin, sterculia, surinabant, synephrine, taranabant, tesofensine, topiramate, viloxazine, xylopropamine, yohimbine, zonisamide, and zylofuramine, and pharmaceutically acceptable salts, solvates, and hydrates thereof.

In some embodiments, the second anti-obesity agent is selected from: 4-methylamphetamine, amfecloral, amfepentorex, amfepramone, a minorex, amphetamine, amphetaminil, atomoxetine, benfluorex, benzphetamine, bupropion, cathine, cathinone, chlorphentermine, ciclazindol, clobenzorex, cloforex, clominorex, clortermine, dexmethylphenidate, dextroamphetamine, dextromethamphetamine, difemetorex, dimethylcathinone, diphemethoxidine, ephedra, ephedrine, ethylamphetamine, etolorex, fenbutrazate, fencamfamine, fenethylline, fenproporex, fludorex, fluminorex, furfenorex, indanorex, khat, levopropylhexedrine, lisdexamfetamine, manifaxine, mazindol, mefenorex, methamphetamine, methylphenidate, pemoline, phendimetrazine, phenethylamine, phenmetrazine, phenpentermine, phentermine, phenylpropanolamine, pipradrol, prolintane, propylhexedrine, pseudoephedrine, pyrovalerone, radafaxine, reboxetine, setazindol, sibutramine, synephrine, taranabant, tesofensine, viloxazine, xylopropamine, and zylofuramine, and pharmaceutically acceptable salts, solvates, and hydrates thereof.

In some embodiments, the second anti-obesity agent is selected from: chlorphentermine, clortermine, phenpentermine, and phentermine, and pharmaceutically acceptable salts, solvates, and hydrates thereof.

In some embodiments the salts of the present invention can be used in combination with an anti-diabetes agent. Anti-diabetes agents include, for example, DPP-IV inhibitors, biguanides, alpha-glucosidase inhibitors, insulin analogues, sulfonylureas, SGLT2 inhibitors, meglitinides, thiazolidinediones, anti-diabetic peptide analogues, and GPR119 agonists.

In some embodiments, the anti-diabetes agent is selected from: sitagliptin, vildagliptin, saxagliptin, alogliptin, linagliptin, phenformin, metformin, buformin, proguanil, acarbose, miglitol, voglibose, tolbutamide, acetohexamide, tolazamide, chlorpropamide, glipizide, glibenclamide, glimepiride, gliclazide, dapagliflozin, remigliflozin, sergliflozin, and 4-[6-(6-methanesulfonyl-2-methyl-pyridin-3-ylamino)-5-methoxy-pyrimidin-4-yloxy]-piperidine-1-carboxylic acid isopropyl ester.

In some embodiments, the anti-diabetes agent is a DPP-IV inhibitor selected from the following compounds and pharmaceutically acceptable salts, solvates, and hydrates thereof: 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one; 1-[2-(3-hydroxyadamant-1-ylamino)acetyl]pyrrolidine-2(S)-carbonitrile; (1S,3S,5S)-2-[2(S)-amino-2-(3-hydroxyadamantan-1-yl)acetyl]-2-azabicyclo[3.1.0]hexane-3-carbonitrile; 2-[6-[3(R)-aminopiperidin-1-yl]-3-methyl-2,4-dioxo-1,2,3,4-tetrahydropyrimidin-1-ylmethyl]benzonitrile; 8-[3(R)-aminopiperidin-1-yl]-7-(2-butynyl)-3-methyl-1-(4-methylquinazolin-2-ylmethyl)xanthine; 1-[N-[3(R)-pyrrolidinyl]glycyl]pyrrolidin-2(R)-yl boronic acid; 4(S)-fluoro-1-[2-[(1R,3S)-3-(1H-1,2,4-triazol-1-ylmethyl)cyclopentylamino]acetyl]pyrrolidine-2(S)-carbonitrile; 1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one; (2S,4S)-2-cyano-4-fluoro-1-[(2-hydroxy-1,1-dimethyl) ethylamino]acetylpyrrolidine; 8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione; 1-((3S,4S)-4-amino-1-(4-(3,3-difluoropyrrolidin-1-yl)-1,3,5-triazin-2-yl)pyrrolidin-3-yl)-5,5-difluoropiperidin-2-one; (R)-2-((6-(3-aminopiperidin-1-yl)-3-methyl-2,4-dioxo-3,4-dihydropyrimidin-1(2H)-yl)methyl)-4-fluorobenzonitrile; 5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide; ((2S,4S)-4-(4-(3-methyl-1-phenyl-1H-pyrazol-5-yl)piperazin-1-yl)pyrrolidin-2-yl)(thiazolidin-3-yl)methanone; (2S,4S)-1-[2-[(4-ethoxycarbonylbicyclo[2.2.2]oct-1-yl)amino]acetyl]-4-fluoropyrrolidine-2-carbonitrile; 6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione; 2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile; (2S)-1-{[2-(5-methyl-2-phenyl-oxazol-4-yl)-ethylamino]-acetyl}-pyrrolidine-2-carbonitrile; (2S)-1-{[1,1-dimethyl-3-(4-pyridin-3-yl-imidazol-1-yl)-propylamino]-acetyl}-pyrrolidine-2-carbonitrile; (3,3-difluoropyrrolidin-1-yl)-((2S,4S)-4-(4-(pyrimidin-2-yl)piperazin-1-yl)pyrrolidin-2-yl)methanone; (2S,4S)-1-[(2S)-2-amino-3,3-bis(4-fluorophenyl)propanoyl]-4-fluoropyrrolidine-2-carbonitrile; (2S,5R)-5-ethynyl-1-{N-(4-methyl-1-(4-carboxy-pyridin-2-yl)piperidin-4-yl)glycyl}pyrrolidine-2-carbonitrile; and (1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine.

In some embodiments, the anti-diabetes agent is an alpha-glucosidase inhibitor selected from the following compounds and pharmaceutically acceptable salts, solvates, and hydrates thereof: (2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal; (2R,3R,4R,5S)-1-(2-hydroxyethyl)-2-(hydroxymethyl)piperidine-3,4,5-triol; and (1S,2S,3R,4S,5S)-5-(1,3-dihydroxypropan-2-ylamino)-1-(hydroxymethyl)cyclohexane-1,2,3,4-tetraol.

In some embodiments, the anti-diabetes agent is a sulfonylurea selected from the following compounds and pharmaceutically acceptable salts, solvates, and hydrates thereof: N-(4-(N-(cyclohexylcarbamoyl)sulfamoyl)phenethyl)-5-methylpyrazine-2-carboxamide); 5-chloro-N-(4-(N-(cyclohexylcarbamoyl)sulfamoyl)phenethyl)-2-methoxybenzamide; and 3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide.

In some embodiments, the anti-diabetes agent is an SGLT2 inhibitor selected from the following compounds and pharmaceutically acceptable salts, solvates, and hydrates thereof: (2S,3R,4R,5S,6R)-2-(4-chloro-3-(4-ethoxybenzyl)phenyl)-6-(hydroxymethyl)tetrahydro-2H-pyran-3,4,5-triol; ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate; and ethyl ((2R,3S,4S,5R,6S)-3,4,5-trihydroxy-6-(2-(4-methoxybenzyl)phenoxy)tetrahydro-2H-pyran-2-yl)methyl carbonate.

In some embodiments, the anti-diabetes agent is a meglitinide selected from the following compounds and pharmaceutically acceptable salts, solvates, and hydrates thereof: (5)-2-ethoxy-4-(2-(3-methyl-1-(2-(piperidin-1-yl)phenyl)butylamino)-2-oxoethyl)benzoic acid; (R)-2-((1r,4R)-4-isopropylcyclohexanecarboxamido)-3-phenylpropanoic acid; and (S)-2-benzyl-4-((3aR,7aS)-1H-isoindol-2(3H,3aH,4H,5H,6H,7H,7aH)-yl)-4-oxobutanoic acid.

In some embodiments, the anti-diabetes agent is a biguanide selected from the following compounds and pharmaceutically acceptable salts, solvates, and hydrates thereof: metformin, phenformin, buformin, and proguanil.

In some embodiments, the anti-diabetes agent is metformin.

In some embodiments, the anti-diabetes agent is a GPR119 agonist selected from the GPR119 agonists disclosed in the following PCT applications: WO2006083491, WO 2008081204, WO2009123992, WO2010008739, WO2010029089, and WO2010149684.

In some embodiments, the anti-diabetes agent is 4-[6-(6-methanesulfonyl-2-methyl-pyridin-3-ylamino)-5-methoxy-pyrimidin-4-yloxy]-piperidine-1-carboxylic acid isopropyl ester.

In some embodiments, the anti-diabetes agent is 5-(4-(4-(3-fluoro-4-(methylsulfonyl)phenoxy)butan-2-yl)piperidin-1-yl)-3-isopropyl-1,2,4-oxadiazole.

Other anti-obesity agents, and anti-diabetes agents including the agents set forth infra, are well known, or will be readily apparent in light of the instant disclosure, to one of ordinary skill in the art. It will be understood that the scope of combination therapy of the salts of the present invention with other anti-obesity agents and with anti-diabetes agents is not limited to those listed above, but includes in principle any combination with any pharmaceutical agent or pharmaceutical composition useful for the treatment of overweight, obese, and diabetic individuals.

One aspect of the present invention pertains to salts of the present invention, characterized in that the salts is administered in conjunction with a second anti-obesity agent as described herein.

One aspect of the present invention pertains to salts of the present invention, characterized in that the salt is administered in conjunction with an anti-diabetes agent as described herein.

One aspect of the present invention pertains to salts of the present invention for use in combination with a second anti-obesity agent for use in weight management.

One aspect of the present invention pertains to salts of the present invention for use in combination with an anti-diabetes agent for use in weight management and the treatment of diabetes.

One aspect of the present invention pertains to methods of weight management in an individual in need thereof, comprising administering to the individual a salt of the present invention and a second anti-obesity agent wherein the salt and the second anti-obesity agent are administered to the individual simultaneously, separately, or sequentially.

One aspect of the present invention pertains to methods of weight management and treating diabetes in an individual in need thereof, comprising administering to the individual a salt of the present invention and an anti-diabetes agent wherein the salt and the anti-diabetes agent are administered to the individual simultaneously, separately, or sequentially.

One aspect of the present invention pertains to methods of weight management in an individual in need thereof, wherein the individual has been or is being treated with a second anti-obesity agent, the method comprising administering to the individual a therapeutically effective amount of a salt of the present invention.

One aspect of the present invention pertains to methods of weight management and treatment of diabetes in an individual in need thereof, wherein the individual has been or is being treated with an anti-diabetes agent, the method comprising administering to the individual a therapeutically effective amount of a salt of the present invention.

One aspect of the present invention pertains to anti-obesity agents, characterized in that the anti-obesity agent is administered in conjunction with a salt of the present invention.

One aspect of the present invention pertains to anti-diabetes agents, characterized in that the anti-diabetes agent is administered in conjunction with a salt of the present invention.

One aspect of the present invention pertains to anti-obesity agents for use in combination with a salt of the present invention for use in weight management.

One aspect of the present invention pertains to anti-diabetes agents for use in combination with a salt of the present invention for use in weight management and the treatment of diabetes.

One aspect of the present invention pertains to methods of weight management in an individual in need thereof, comprising administering to the individual an anti-obesity agent and a salt of the present invention wherein the anti-obesity agent and the salt are administered to the individual simultaneously, separately, or sequentially.

One aspect of the present invention pertains to methods of weight management and treating diabetes in an individual in need thereof, comprising administering to the individual an anti-diabetes agent and a salt of the present invention wherein the anti-diabetes agent and the salt are administered to the individual simultaneously, separately, or sequentially.

One aspect of the present invention pertains to methods of weight management in an individual in need thereof, wherein the individual has been or is being treated with a salt of the present invention, the method comprising administering to the individual a therapeutically effective amount of a second anti-obesity agent.

One aspect of the present invention pertains to methods of weight management and treatment of diabetes in an individual in need thereof, wherein the individual has been or is being treated with a salt of the present invention, the method comprising administering to the individual a therapeutically effective amount of an anti-diabetes agent.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

The following examples are provided to further define the invention without, however, limiting the invention to the particulars of these examples. The compounds and salts thereof described herein, supra and infra, are named according to the CS ChemDraw Ultra Version 7.0.1, AutoNom version 2.2, or CS ChemDraw Ultra Version 9.0.7. In certain instances common names are used and it is understood that these common names would be recognized by those skilled in the art.

Powder X-ray Diffraction (PXRD) studies were conducted using an X′Pert PRO MPD powder diffractometer (PANalytical, Inc.; EQ0233) with a Cu source set at 45 kV and 40 mA, Cu(Kα) radiation and an X′Celerator detector. Samples were placed on a PXRD sample plate either as-is or ground slightly to reduce the size of large particles or crystals. Data were collected with the samples spinning from 5° to 40 °2θ. Data were analyzed by X′Pert Data Viewer software, version 1.0a, to determine crystallinity and/or crystal form, and by X′Pert HighScore software, version 1.0b, to generate the tables of PXRD peaks.

Differential scanning calorimetry (DSC) studies were conducted using a TA Instruments, Q2000 (EQ1980) at heating rate 10° C./min. The instruments were calibrated by the vendor for temperature and energy using the melting point and enthalpy of fusion of an indium standard.

Thermogravimetric analyses (TGA) were conducted using a TA Instruments TGA Q5000 (EQ1982) at heating rate 10° C./min. The instrument was calibrated by the vendor using Alumel and Nickel Curie points for the furnace temperature and a standard weight for the balance.

Dynamic moisture-sorption (DMS) studies were conducted using a dynamic moisture-sorption analyzer, VTI Corporation, SGA-100, equipment #0228. Samples were prepared for DMS analysis by placing 5 mg to 20 mg of a sample in a tared sample holder. The sample was placed on the hang-down wire of the VTI balance. A drying step was run, typically at 40° C. and 0.5-1% RH for 1-2 h. The isotherm temperature is 25° C. Defined % RH holds typically ranged from 10% RH to 90% RH or 95% RH, with intervals of 10 to 20% RH. A % weight change smaller than 0.010% over a specified number of minutes (typically 10-20), or up to 2 h, whichever occurs first, is required before continuing to the next % RH hold. The water content of the sample equilibrated as described above was determined at each % RH hold.

If saturated in water with excess solid, a deliquescing compound or salt thereof equilibrated in a closed system at a given temperature produces a % RH in that closed system that is equal to its deliquescing % RH (DRH) at that temperature. Fractional relative humidity is equal to water activity (a_{w}) in the vapor phase and at equilibrium in a closed system, the a_{w} in an aqueous solution is equal to the aw in the vapor phase above the solution (see Equation 1).

$$\begin{matrix}\frac{D R H}{100 \%} = \frac{\% R H}{100 \%} ( above enclosed sat aq {sol}^{′} n at equil ) \\ = {a}_{w} ( vapor ) \\ = {a}_{w} ( liquid )\end{matrix} \qquad Equation 1$$

A water activity meter was used to measure DRH for selected salts described herein. The instrument used for this study is a Decagon Devices AquaLab 4TE water activity meter, equipment #2169. This instrument is designed with temperature control and a small headspace above the enclosed sample to establish equilibrium between solution and vapor phases quickly. Measured a_{w} values at 25° C. for samples of aqueous-saturated Compound 1 salts with excess solid were multiplied by 100% to get DRH values in % RH.

Acquity ultra performance liquid chromatography (UPLC) from Waters was used for solubility and stoichiometry determination. Instrument number is SY-EQ 1889. UPLC was equipped with Acquity PDA detector. UPLC mobile phase solvent A was 0.1% TFA in DI-water, solvent B was 0.1% TFA in acetonitrile. The mobile phase gradient as shown in the table below:

| Time (min) | Flow (mL/min) | % A | % B | Curve |
|---|---|---|---|---|
| | 0.600 | 95.0 | 5.0 | |
| 2.00 | 0.600 | 5.0 | 95.0 | 6 |
| 2.50 | 0.600 | 5.0 | 95.0 | 6 |
| 2.75 | 0.600 | 95.0 | 5.0 | 1 |
| 5.00 | 0.000 | 95.0 | 5.0 | 11 |

Column temperature was 40±5° C. Acquity UPLC® HSS T3 1.8 μm, 2.1×50 mm column was used.

A known amount of sample was dissolved in water and analyzed by UPLC. The weight percent of Compound 1 in the salt samples was determined by comparing the UV signal to that of a standard, Compound 1 hydrochloride salt hemihydrate, or Compound 1 free base. The percentage of Compound 1 or the percentage of the counterion determined was compared to the theoretical values to establish the stoichiometry.

### Example 1

### Preparation of Form I of (R)-8-Chloro-1-methyl-2,3,4,5-tetrahydro-1H-3-benzazepine Bisulfate Salt (Compound 1 Bisulfate Salt, Form I)

The title salt, was prepared by drop-wise addition of 1 mole equivalent of concentrated sulfuric acid to a solution of (R)-8-chloro-1-methyl-2,3,4,5-tetrahydro-1H-3-benzazepine free base in either isopropyl acetate or acetonitrile with vigorous stirring. Precipitation occurred immediately and the suspension was allowed to stir for 1 to 2 days. The resulting solid was recovered by filtration.

The title salt was an anhydrous crystalline material with melting onset ˜162° C. It was non-hygroscopic by DMS up to and including 70% RH, but picked up significant water between 70 and 90% RH. The DRH was determined by water activity measurement of saturated aqueous solution with excess solid to be 83% RH at 25° C. Post-DMS PXRD analysis showed no change in the crystalline phase.

A known amount of the title salt was dissolved in water and analyzed by UPLC. The amount of (R)-8-chloro-1-methyl-2,3,4,5-tetrahydro-1H-3-benzazepine in the sample was determined to be 70.6%. This is slightly higher than the theoretical amount (66.6%).

The title salt was determined visually to be “very soluble” in water per the USP categorization (<1 mL water needed to dissolve 1 g.) The final pH was ˜0.

The powder X-ray diffraction pattern of the title salt is shown in FIG. 5. Thermal analysis (TGA and DSC) of the title salt is shown in FIG. 6. DMS analysis of the title salt is shown in FIG. 7.

### Example 2

### Preparation of Form I of (R)-8-Chloro-1-methyl-2,3,4,5-tetrahydro-1H-3-benzazepine Hemisulfate Salt Hydrate (Compound 1 Hemisulfate Salt Hydrate, Form I)

The title salt was prepared by the drop-wise addition of 0.5 mole equivalent of concentrated sulfuric acid to a solution of (R)-8-chloro-1-methyl-2,3,4,5-tetrahydro-1H-3-benzazepine free base in either isopropyl acetate or acetonitrile with vigorous stirring. Precipitation occurred immediately and the suspension was allowed to stir for 1 to 2 days. The resulting yellow solid was recovered by filtration. Acetone was added to the solid followed by sufficient water to cause dispersal (<5%). This mixture was slurried for 4 h and the solid was collected by centrifuge filtration (10,000 rpm for 1 min). The filtrate contained an oil droplet and the filter cake had a small amount of color at the bottom. The white upper portion of the filter cake was removed and air-dried overnight to leave the title salt as a white solid.

Form I of Compound 1 hemisulfate salt hydrate, was a hydrated crystalline material with a dehydration onset temperature below 50° C. by TGA scanned at 10° C./min. The weight loss by TGA depended on the sample and perhaps the humidity on the day of analysis. The range for samples analyzed was 2.9% to 3.3%. These values are less than hemihydrate stoichiometry (3.55% water by weight). Although close to a hemihydrate with respect to Compound 1, the onset of weight loss was very low and thus this salt appears to be a channel hydrate.

Form I of Compound 1 hemisulfate salt hydrate was very soluble in water, per USP categorization (<1 mL water needed to dissolve 1 g). The final pH was 2.

Form I of Compound 1 hemisulfate salt hydrate was slightly hygroscopic by DMS up to 80% RH, (˜2% water up to and including the 80% RH hold). DMS also showed the salt picked up significantly more water at the 90% RH hold, indicating the salt was deliquescent between 80 and 90% RH. The drying step during DMS analysis resulted in partial dehydration of Compound 1 hemisulfate salt hydrate. This dried-off water is essentially recovered by the first humidity hold at 10% RH. The hysteresis does not correspond to a new hydrate, but rather it represents outer crust formation during desorption, which leads to limited diffusion of water from the sample during the desorption cycle. This phenomenon is not uncommon for deliquescing compounds. Post-DMS PXRD analysis showed no change in the crystalline phase.

The DRH was determined by water activity measurement of saturated aqueous solution with excess solid to be 86% RH at 25° C.

A known amount of Form I of Compound 1 hemisulfate salt hydrate was dissolved in water and analyzed by UPLC. The amount of Compound 1 in the salt sample was determined to be 80.7%. This is in agreement with the theoretical value (80.5%) in Compound 1 hemisulfate salt hydrated with 0.41 moles of water based on TGA data.

The powder X-ray diffraction pattern of the title salt is shown in FIG. 8. DSC of the title salt is shown in FIG. 9. Thermal analyses (TGA) of the title salt are shown in FIGS. 9 and 10. DMS analysis of the title salt is shown in FIG. 11.

### Example 3

### Preparation of Form I of (R)-8-Chloro-1-methyl-2,3,4,5-tetrahydro-1H-3-benzazepine Mesylate Salt (Compound 1 Mesylate Salt, Form I)

The title salt was prepared by the dropwise addition of one equivalent of methanesulfonic acid (99.5%) to a solution of (R)-8-chloro-1-methyl-2,3,4,5-tetrahydro-1H-3-benzazepine free base in acetonitrile, or isopropyl acetate with vigorous stirring. Crystallization occurred either immediately or within 24 hours after the solution was heated to ˜60° C. and then allowed to cool to RT while stirring.

The title salt had a melting onset about 178° C. It appeared to hold a small amount of residual solvent by TGA, losing about 0.12% weight just prior to the melting onset.

The title salt was non-hygroscopic out to and including the 90% RH hold at 25° C., picking up about 0.5% in weight. However, at 95% RH it picked up about 3.2% weight. This is consistent with the DRH, 93.8% RH at 25° C., determined by water activity measurement of a sample saturated in water with excess solid.

A known amount of the title salt was dissolved in water and analyzed by UPLC. The amount of Compound 1 in the sample was determined to be 72.6%. This is slightly higher than the theoretical value, 67.1%.

The aqueous solubility of the title salt was determined by UPLC to be 612 mg/mL, with a final pH of 1.

The powder X-ray diffraction pattern of the title salt is shown in FIG. 12. Thermal analysis (TGA and DSC) of the title salt is shown in FIG. 13. DMS analysis of the title salt is shown in FIG. 14.

### Example 4

### Preparation of Form I of (R)-8-Chloro-1-methyl-2,3,4,5-tetrahydro-1H-3-benzazepine Hydrobromide Salt Hemihydrate (Compound 1 Hydrobromide Salt Hemihydrate, Form I)

The title salt was prepared by the dropwise addition of one equivalent of aqueous HBr (˜48%) to a solution of (R)-8-chloro-1-methyl-2,3,4,5-tetrahydro-1H-3-benzazepine free base in isopropyl acetate, acetonitrile, or ethyl acetate with vigorous stirring. The product readily precipitated from the reaction in isopropyl acetate. In acetonitrile the solvent was evaporated to near dryness to obtain a solid.

In ethyl acetate, seeds were added and the reaction was allowed to stir unstoppered to initiate crystallization. The reaction was then closed and stirring was continued to afford a yellow suspension. The suspension was filtered and the solid was washed with cold ethyl acetate. The resulting white solid was under nitrogen at ˜38° C., and held overnight at 25° C./75% RH.

The title salt was a hemihydrate with a dehydration onset at about 72.5° C. by TGA. The water content was lower than the theoretical value for a hemihydrate (3.15%) when the TGA integration was carried out to the perceived end of the DSC dehydration endotherm. An upper integration limit of about ˜175° C. was needed to achieve a weight loss equivalent to 0.5 moles of water.

The title salt was non-hygroscopic, picking up ˜0.3% weight out to and including the 90% RH hold at 25° C. Analysis of a saturated aqueous solution with excess solid by water activity meter showed the title salt to have a very high DRH of 98% RH at 25° C.

Form I of Compound 1 hydrobromide salt hemihydrate is isostructural to Form III of Compound 1 hydrochloride salt hemihydrate based on a very similar PXRD pattern (see WO2006/069363) and the same hydration state as determined by Karl-Fischer analysis (3.18±0.04%).

A known amount of the title salt was dissolved in water and analyzed by UPLC. The amount of Compound 1 in the sample was determined to be 71.8%. This is in agreement with the theoretical value, 68.5%. The solubility in water was 404 mg/mL as determined by UPLC. The final pH was 5.71.

The powder X-ray diffraction pattern of the title salt is shown in FIG. 15. Thermal analysis (TGA and DSC) of the title salt is shown in FIG. 16. DMS analysis of the title salt is shown in FIG. 17.

### Example 5

### Preparation of Form I of (R)-8-Chloro-1-methyl-2,3,4,5-tetrahydro-1H-3-benzazepine Nitrate Salt (Compound 1 Nitrate Salt, Form I)

The title salt was prepared by dropwise addition of aqueous HNO₃ to a solution of (R)-8-chloro-1-methyl-2,3,4,5-tetrahydro-1H-3-benzazepine free base in isopropyl acetate or acetonitrile with vigorous stirring.

The title salt was an anhydrous material with a melting onset of about 124° C. It was very slightly hygroscopic, picking up ˜1% weight by DMS analysis out to and including the 90% RH hold at 25° C. The DRH by water activity measurement of a saturated solution with excess solid was 99% RH at 25° C.

A known amount of the title salt was dissolved in water and analyzed by UPLC. The amount of Compound 1 in the sample was determined to be 78.6%. This is in good agreement with the theoretical value, 75.6%. The solubility in water was 1109 mg/mL as determined by UPLC. The final pH was 5.14.

The powder X-ray diffraction pattern of the title salt is shown in FIG. 18. Thermal analysis (TGA and DSC) of the title salt is shown in FIG. 19. DMS analysis of the title salt is shown in FIG. 20.

### Example 6

### Preparation of Form I of (R)-8-Chloro-1-methyl-2,3,4,5-tetrahydro-1H-3-benzazepine Sesqui-oxalate Salt-Cocrystal (Compound 1 Sesqui-oxalate Salt-Cocrystal, Form I)

The title salt was prepared by addition of oxalic acid (0.5 eq.) to a solution of (R)-8-chloro-1-methyl-2,3,4,5-tetrahydro-1H-3-benzazepine in isopropyl acetate. The stoichiometry of the resulting solid was 1 mole of Compound 1 to 1.5 moles of oxalic acid.

The title salt showed by DSC an apparent melt, followed immediately by recrystallization, and followed immediately by melting. The initial endotherm had an onset of 105° C.; the second endotherm melt had a melting onset of 111° C. The title salt was slightly hygroscopic, picking up about 1.4% weight out to and including the 90% RH hold at 25° C.

A known amount of the title salt was dissolved in water and analyzed by UPLC. The amount of Compound 1 in the sample was 60.5%. This is in fair agreement with the theoretical amount for a sesqui-oxalate (salt-cocrystal), 59.2%. Aqueous solubility was determined to be >500 mg/mL with a final pH 4.95.

The powder X-ray diffraction pattern of the title salt is shown in FIG. 21. Thermal analysis (TGA and DSC) of the title salt is shown in FIG. 22. DMS analysis of the title salt is shown in FIG. 23.

### Example 7

### Preparation of Form I of (R)-8-Chloro-1-methyl-2,3,4,5-tetrahydro-1H-3-benzazepine Adipate Salt (Compound 1 Adipate Salt, Form I)

The title salt was prepared by addition of adipic acid (0.5-1 eq.) in acetone to a solution of (R)-8-chloro-1-methyl-2,3,4,5-tetrahydro-1H-3-benzazepine at ˜62° C. Precipitation occurred within 5 min and the suspension was allowed to cool to ambient temperature with stirring.

DSC and TGA analyses of the title salt showed that it was an anhydrous salt with multiple endothermic events starting at onset temperatures between 104° C. and 107° C. It was hygroscopic at 70% RH and above, picking up 10.87% weight out to and including the 90% RH hold at 25° C.

Aqueous solubility of the title salt was 964 mg free base/mL, which resulted in a final of pH 5.1.

The powder X-ray diffraction pattern of the title salt is shown in FIG. 24. Thermal analysis (TGA and DSC) of the title salt is shown in FIG. 25. DMS analysis of the title salt is shown in FIG. 26.

### Example 8

### Preparation of Form I of (R)-8-Chloro-1-methyl-2,3,4,5-tetrahydro-1H-3-benzazepine Malonate Salt (Compound 1 Malonate Salt, Form I)

The title salt was prepared by addition of malonic acid (1 eq.) to a solution of (R)-8-chloro-1-methyl-2,3,4,5-tetrahydro-1H-3-benzazepine in isopropyl acetate.

The title salt was an anhydrous non-hygroscopic salt, picking up ˜0.2% weight out to and including the 90% RH hold at 25° C. DRH=95.1% RH and the melting onset was 143.0° C. The solubility in water was 712 mg/mL with a final pH of 3.8.

The title salt displayed a melting onset between about 143-145° C. The TGA showed complete volatilization of the salt after melting.

The title salt was non-hygroscopic, picking up ˜0.2% weight out to and including the 90% RH hold at 25° C. was measured by water activity determination for a saturated aqueous solution with excess solid to be 95.1% RH at 25° C.

A known amount of the title salt was dissolved in water and analyzed by UPLC. The amount of Compound 1 in the sample was 68.5%. This is slightly higher than the theoretical amount, 65.3%. Aqueous solubility of the title salt was 712 mg/mL. The final pH was 3.8.

The powder X-ray diffraction pattern of the title salt is shown in FIG. 27. Thermal analysis (TGA and DSC) of the title salt is shown in FIG. 28. DMS analysis of the title salt is shown in FIG. 29.

### Example 9

### Preparation of Form I of (R)-8-Chloro-1-methyl-2,3,4,5-tetrahydro-1H-3-benzazepine Hemimalonate Salt (Compound 1 Hemimalonate Salt, Form I)

The title salt was prepared by addition of malonic acid (0.5 eq.) to a solution of (R)-8-chloro-1-methyl-2,3,4,5-tetrahydro-1H-3-benzazepine in isopropyl acetate.

The title salt had a melting onset at about 135-136° C. The TGA showed an anhydrous salt with complete volatilization after melting.

A known amount of the title salt was dissolved in water and analyzed by UPLC. The amount of Compound 1 in the sample was 76.9%. This is slightly lower than but in fair agreement with the theoretical value for an anhydrous hemimalonate salt, 79.0%. Aqueous solubility of the title salt was 772 mg/mL. The final pH of a near saturated solution of this salt was 6.0.

The powder X-ray diffraction pattern of the title salt is shown in FIG. 30. Thermal analysis (TGA and DSC) of the title salt is shown in FIG. 31.

### Example 10

### Preparation of Form I of (R)-8-Chloro-1-methyl-2,3,4,5-tetrahydro-1H-3-benzazepine Glycolate Salt (Compound 1 Glycolate Salt, Form I)

The title salt was prepared by the addition of one equivalent of glycolic acid to a solution of (R)-8-chloro-1-methyl-2,3,4,5-tetrahydro-1H-3-benzazepine in ethyl acetate or acetone at 60° C. Glycolic acid, at 60° C., was added dropwise, in the corresponding solvent, with vigorous stirring. Precipitation occurred immediately and the suspension was allowed to cool and stir overnight. The resulting solid was recovered by filtration and air-dried in a fume hood overnight.

A known amount of the title salt was dissolved in methanol and analyzed by UPLC. The percentage of Compound 1 in the salt sample was determined to be 63.7%. This is slightly lower than the theoretical percentage of Compound 1 in an anhydrous Compound 1 glycolate salt (72.01%).

Solubility of Compound 1 glycolate salt in water was determined by UPLC to be >49.8 mg/mL, with a final pH of 6.89.

The powder X-ray diffraction pattern of the title salt is shown in FIG. 32. Thermal analysis (TGA and DSC) of the title salt is shown in FIG. 33. DMS analysis of the title salt is shown in FIG. 34.

### Example 11

### Preparation of (R)-8-Chloro-1-methyl-2,3,4,5-tetrahydro-1H-3-benzazepine Hydrochloride Salt Hemihydrate, Form III

### Method 1

### Step A: Preparation of 8-Chloro-1-methyl-2,3,4,5-tetrahydro-1H-3-benzazepine

2-Chloro-N-(4-chlorophenethyl)propan-1-amine hydrochloride (about 460 kg, 1.71 kmol, 1.00 eq.), aluminum chloride (about 336 kg, 2.52 kmol, 1.47 eq.), and 1,2-dichlorobenzene (about 1321 kg) are charged to a vessel vented to a caustic scrubber. The mixture is then stirred and heated at about 126° C. under nitrogen for about 16 h. The resulting Friedel-Crafts reaction mixture is then cooled. Silica gel and purified water (about 736 kg) are charged to a second vessel. The cooled Friedel-Crafts reaction mixture is then added to the aqueous silica gel slurry stirred and cooled in the second vessel. The stirred quench mixture is filtered at about 55° C., and the silica gel filter cake is washed with purified water (about 368 kg). Optionally, some or all of this purified water is used to rinse the quench vessel into the filter.

The mother and wash liquor filtrates are combined in a vessel and are cooled with stirring to about 22° C. Stirring is then stopped, and upon settling, three phases separate. The brown, lowest phase consists mostly of 1,2-dichlorobenzene and is drained. The lower of the remaining two phases, which is the middle phase of the original three-phase mixture, contains most of the product. The topmost phase is a turbid water phase containing a smaller amount of the product. These upper two phases are partitioned between cyclohexane (about 506 kg) and enough aqueous sodium hydroxide solution, approx. 30 wt %, to achieve an aqueous phase pH of at least 12. The cyclohexane phase is washed with water (at least 300 kg) at about 57° C. and then evaporated at reduced pressure to provide crude 8-chloro-1-methyl-2,3,4,5-tetrahydro-1H-3-benzazepine as an oil.

### Step B: Preparation of (R)-8-Chloro-1-methyl-2,3,4,5-tetrahydro-1H-3-benzazepine Hemitartrate Salt

Acetone (about 848 kg) is added to the crude 8-chloro-1-methyl-2,3,4,5-tetrahydro-1H-3-benzazepine prepared in Step A. The vessel contents are stirred and heated to about 45° C. To the resulting solution is added a solution of L-(+)-tartaric acid (about 57.0 kg, 380 mol, 0.222 eq.) in purified water (about 98.0 kg) while the stirred vessel contents are maintained at about 45° C. Stirring is continued for about 20 min. (R)-8-chloro-1-methyl-2,3,4,5-tetrahydro-1H-3-benzazepine hemitartrate salt seed crystals are then optionally added to initiate nucleation. Stirring is continued, and more acetone is added. The resulting suspension is then cooled to about 2° C. The resulting precipitate is collected by centrifugation and washed with acetone (about 440 kg), a portion of which is optionally used to rinse the crystallization vessel into the centrifuge. The washed solid is discharged from the centrifuge, mixed with acetone (about 874 kg) and the mixture is stirred and heated to reflux. While reflux is maintained, purified water (at least 329 kg) is added until complete dissolution is achieved at reflux. The resulting mixture is stirred at reflux and then cooled to about 2° C. over about 2.5 hours. The resulting precipitate is collected by centrifugation and washed with acetone (about 184 kg), a portion of which is optionally used to rinse the crystallization vessel into the centrifuge. The washed solid is discharged from the centrifuge and dried at elevated temperature under reduced pressure to provide (R)-8-chloro-1-methyl-2,3,4,5-tetrahydro-1H-3-benzazepine hemitartrate. The yield range is 100 kg to 158 kg.

### Step C: Preparation of (R)-8-Chloro-1-methyl-2,3,4,5-tetrahydro-1H-3-benzazepine Hydrochloride Salt Hemihydrate, Form III

Purified water (about 740 kg) is added to a stirred mixture of (R)-8-chloro-1-methyl-2,3,4,5-tetrahydro-1H-3-benzazepine hemitartrate from Step B (about 247 kg after correction for assay, 912 mol, 1.00 eq.), potassium carbonate (about 151 kg, 1093 mol, 1.20 eq.), and ethyl acetate (about 663 kg). The mixture is maintained at about 15° C. during the addition, after which it is stirred and then allowed to settle. The lower (aqueous) phase is drained to waste disposal. Purified water (about 740 kg) is added to the upper (organic) phase, and the resulting mixture is stirred at about 22° C. and then allowed to settle. The lower (aqueous) phase is drained to waste disposal.

Solvent is removed from the upper (organic) phase by vacuum distillation at about 40° C. to provide (R)-8-chloro-1-methyl-2,3,4,5-tetrahydro-1H-3-benzazepine as the distillation residue. Ethyl acetate (about 1050 kg) is added, and the mixture is stirred to achieve dissolution. If the water content of the resulting solution is found by Karl Fischer analysis to exceed 1.51 wt %, the procedure of this paragraph is repeated.

Through a polishing filter into a crystallization vessel is added purified water in the approximate amount calculated to provide a water concentration of 1.0 wt % in the (R)-8-chloro-1-methyl-2,3,4,5-tetrahydro-1H-3-benzazepine solution after the final ethyl acetate dilution. The solution is then filtered through the same polishing filter into the crystallization vessel. The vessel in which the (R)-8-chloro-1-methyl-2,3,4,5-tetrahydro-1H-3-benzazepine had been prepared is rinsed with additional fresh ethyl acetate (about 644 kg), and the rinse is filtered through the same polishing filter into the crystallization vessel.

The water content of the solution in the crystallization vessel is determined by Karl Fischer analysis. If the water content is about 0.8 wt % to about 1.2 wt % (0.5 wt % to 1.5 wt % non-critical range), then processing resumes at the beginning of the next paragraph. If the water content is too low, additional purified water is added through the polishing filter. If the water content is too high, then solvent is removed by vacuum distillation, purified water (about 18 kg) is added through the polishing filter, and ethyl acetate (about 1800 kg) is added through the polishing filter. In either case, the resulting solution is tested for water content.

As the contents of the crystallization vessel are stirred, hydrogen chloride gas (about 3.3 kg, 91 mol, 0.10 eq.) is added to the vessel head space. (R)-8-Chloro-1-methyl-2,3,4,5-tetrahydro-1H-3-benzazepine hydrochloride hemihydrate seed crystals are then added to initiate nucleation. Additional hydrogen chloride gas is then added to the vessel head space until the pH of the reaction mixture drops to and remains at about 5 or less. The precipitated product is collected by centrifugation and washed with filtered ethyl acetate (about 552 kg). The precipitate is dried under reduced pressure to provide the title salt. The yield range is 184 kg to 217 kg, which is 84% to 99% of theoretical uncorrected for seed charge and 83% to 98% of theoretical corrected for seed charge.

### Method 2

### Step A: Preparation of 8-Chloro-1-methyl-2,3,4,5-tetrahydro-1H-3-benzazepine

1,2-Dichlorobenzene (about 1522 kg), 2-chloro-N-(4-chlorophenethyl)propan-1-amine hydrochloride (about 530 kg, 1.97 kmol, 1.00 eq.), and aluminum chloride (about 387 kg, 2.90 kmol, 1.47 eq.) are charged to a vessel vented to a caustic scrubber. The mixture is then stirred and heated at about 126° C. under nitrogen for about 16 h. The resulting Friedel-Crafts reaction mixture is then cooled. Purified or potable water (about 1060 kg) and silica gel are charged to a second vessel. The cooled Friedel-Crafts reaction mixture is then added to the aqueous silica gel slurry stirred and cooled in the second vessel. The stirred quench mixture is filtered at about 58° C., and the silica gel filter cake is washed with purified or potable water (about 212 kg). Optionally, some or all of this water may be used to rinse the quench vessel into the filter. The mother and wash liquor filtrates are combined in a vessel and are cooled with stirring to about 22° C. Stirring is then stopped, and upon settling, three phases separate. The brown lowest phase consists mostly of 1,2-dichlorobenzene and is drained to solvent regeneration. The lower of the remaining two phases, which is the middle phase of the original three-phase mixture, contains most of the product. The topmost phase is a turbid water phase containing a smaller amount of the product. These upper two phases are partitioned between cyclohexane (about 583 kg) and enough aqueous sodium hydroxide solution, approx. 30 wt %, to achieve an aqueous phase pH of at least about 13. The cyclohexane phase is washed with purified or potable water (about 1272 kg) at about 57° C. and then distilled at reduced pressure to remove solvent and provide crude title compound, an oil, as the distillation residue.

### Step B: Preparation of (R)-8-Chloro-1-methyl-2,3,4,5-tetrahydro-1H-3-benzazepine Hemitartrate

Acetone (about 977 kg) is added to the crude 8-chloro-1-methyl-2,3,4,5-tetrahydro-1H-3-benzazepine prepared in Step A. The vessel contents are stirred and heated to about 45° C. To the resulting solution is added a solution of L-(+)-tartaric acid (about 66 kg, 440 mol, 0.223 eq.) in purified or potable water (about 113 kg) while the stirred vessel contents are maintained at about 45° C. About half way through the tartaric acid addition, (R)-8-chloro-1-methyl-2,3,4,5-tetrahydro-1H-3-benzazepine hemitartrate seed crystals are added to the solution to achieve cloudiness and to initiate nucleation. Stirring is continued, and more acetone is added. The resulting suspension is then cooled to about 2° C. The resulting precipitate is collected by centrifugation and washed with acetone (about 508 kg), a portion of which is optionally used to rinse the crystallization vessel into the centrifuge. The washed solid is mixed with acetone (about (1007 kg) and the mixture is stirred and heated to reflux. While reflux is maintained, purified or potable water (at least about 392 kg) is added until complete dissolution is achieved at reflux. The resulting mixture is stirred at reflux and then cooled to about 2° C. over about 2.5 h. The resulting precipitate is collected by centrifugation and washed with acetone (about 212 kg), a portion of which is optionally used to rinse the crystallization vessel into the centrifuge. The washed solid is discharged from the centrifuge and dried at elevated temperature under reduced pressure to provide the title salt.

### Step C: Preparation of (R)-8-Chloro-1-methyl-2,3,4,5-tetrahydro-1H-3-benzazepine Hydrochloride Salt Hemihydrate, Form III

Purified water (about 779 kg) is combined with (R)-8-chloro-1-methyl-2,3,4,5-tetrahydro-1H-3-benzazepine hemitartrate from Step B (about 260 kg after correction for assay, 960 mol, 1.00 eq.), potassium carbonate (about 159 kg, 1150 mol, 1.20 eq.), and ethyl acetate (about 698 kg) with stirring at about 15° C. The resulting mixture is stirred and then allowed to settle. The lower (aqueous) phase is drained to waste disposal. Purified water (about 779 kg) is added to the upper (organic) phase, and the resulting mixture is stirred at about 22° C. and then allowed to settle. The lower (aqueous) phase is drained to waste disposal.

Solvent is removed from the upper (organic) phase by vacuum distillation with the jacket temperature increasing to about 60° C. (R)-8-chloro-1-methyl-2,3,4,5-tetrahydro-1H-3-benzazepine, an oil, is obtained as the distillation residue. Ethyl acetate (about 1105 kg) is added, and the mixture is stirred to achieve dissolution. If the water content of the resulting solution is found by Karl Fischer analysis to exceed 1.51 wt %, the procedure of this paragraph is repeated.

The solution in is then filtered through a polishing filter into a crystallization vessel. The vessel in which the (R)-8-chloro-1-methyl-2,3,4,5-tetrahydro-1H-3-benzazepine had been prepared is then rinsed with additional ethyl acetate (about 122 kg) through the same polishing filter into the crystallization vessel. To the crystallization vessel is then added purified water in the approximate amount calculated to provide a water concentration of 1.0 wt % in the solution after the final ethyl acetate dilution. Ethyl acetate (about 556 kg) is then added to the crystallization vessel, and the resulting mixture is stirred. The water content of the solution in the crystallization vessel is determined by Karl Fischer analysis. If the water content is about 0.8 wt % to about 1.2 wt % (0.5 wt % to 1.5 wt % qualified range), then processing resumes at the beginning of the next paragraph. If the water content is too low, additional purified water is added. If the water content is too high, then solvent is removed by vacuum distillation, and purified water and ethyl acetate are added. In either case, the resulting solution is retested for water content.

As the contents of the crystallization vessel are stirred, hydrogen chloride gas (about 3.5 kg, 96 mol, 0.10 eq.) is added to the vessel head space. (R)-8-chloro-1-methyl-2,3,4,5-tetrahydro-1H-3-benzazepine hydrochloride hemihydrate seed crystals are then added to initiate nucleation. Additional hydrogen chloride gas is then added to the vessel head space until the pH of the reaction mixture drops to and remains at about 3 or less. The precipitated product is collected by centrifugation and washed with ethyl acetate (about 580 kg) to provide the title salt (about 221 kg), which is dried in a tray or tumble dryer (such as a double cone dryer) under reduced pressure at a jacket temperature of about 26° C.

### Method 3

### Step A: Preparation of 8-Chloro-1-methyl-2,3,4,5-tetrahydro-1H-3-benzazepine

To a reactor equipped with overhead agitation, jacket temperature control, a nitrogen inlet, and a caustic scrubber vent were charged, in the specified order, 2-chloro-N-(4-chlorophenethyl)propan-1-amine hydrochloride (1.00 kg, 3.72 mol), aluminum chloride (0.745 kg, 5.58 mol), and 1,2-dichlorobenzene (2.88 kg). The stirred reactor contents were heated to 125-130° C., and stirring was continued at that temperature for 14-18 h. At 60-70° C., a dark colored solution was obtained. After reaction completion (<1.0% starting material by HPLC peak area) had been verified, the stirred reactor contents were cooled to 30-35° C. To a second reactor vented to a caustic scrubber was charged purified water (1.60 L) and silica gel (0.160 kg). The Friedel-Crafts reaction mixture was transferred from the first reactor to the second reactor sufficiently slowly to maintain the stirred contents of the second reactor at <60° C. After the transfer is completed, the next step may be executed without any hold period. The silica gel was filtered on a medium to coarse filter element at 55-60° C., and the filtered solids were subsequently washed with purified water (800 mL) preheated to 50-60° C. The combined mother and wash liquor filtrates were cooled to 20-25° C. with vigorous agitation. Then the stirring was stopped, and the phases were allowed to separate at 20-25° C. (Process volume peaked at this point at 5.68 L). Three phases separated after 1-2 hours of standing. The lowest layer was drained to waste disposal. This dark layer consisted mostly of 1,2-dichlorobenzene (1.64 kg, 1.33 L) at pH 3-4. About 1% of the product was lost to this layer. The remaining two phases were allowed to stand without agitation for another 2-4 h. The lower layer was drained and saved (Layer A). This light colored phase (2.64 kg, 2.00 L, pH 2-3) contained ˜90% 8-chloro-1-methyl-2,3,4,5-tetrahydro-1H-benzazepine. The upper layer (2.24 kg of a turbid water phase at pH 0-1) contains ˜1-4% 8-chloro-1-methyl-2,3,4,5-tetrahydro-1H-benzazepine and remained in the reactor for back-extraction. The reactor was charged with cyclohexane (1.10 kg) and then 30% aqueous NaOH (2.44 kg, 18.3 mol). The resulting mixture (5.60 L) was stirred vigorously for 30 min at room temperature. The stirring was stopped, and the phases were allowed to separate for 25-40 min. If the pH of the lower (aqueous) phase was ≧13, it was drained to waste disposal. Otherwise, more 30% aqueous NaOH was added, and this extraction was repeated. At pH 14, the aqueous phase contains <0.1% 8-chloro-1-methyl-2,3,4,5-tetrahydro-1H-benzazepine free base. The remaining upper (organic) phase from the reactor was drained and saved (Layer B). The reactor was rinsed with purified water and followed by a suitable organic solvent to remove residual salts. The lower, light-colored product phase (the middle of the original three phases, Layer A) and the upper phase (organic, Layer B) were returned to the reactor. To the stirred reactor contents was added 30% aqueous NaOH (1.60 kg, 12.0 mol). The reactor contents were stirred vigorously for 0.5 hours. The stirring was discontinued and the phases were allowed to separate over 15-30 minutes. The lower (aqueous) layer was drained to waste disposal. To the upper (organic) phase remaining in the reactor was added purified water (2.40 kg). The reactor contents were stirred vigorously at 60-65° C. for 0.5 h. The stirring was discontinued, and the phases were allowed to separate at 60-65° C. over 1.5-2 h. The lower (aqueous) layer was drained to waste disposal. With a reactor jacket temperature of 55-60° C., solvent from the upper (organic) layer was removed by vacuum distillation at pressures starting at 115-152 ton and falling to 40 torr. The crude product, 8-chloro-1-methyl-2,3,4,5-tetrahydro-1H-benzazepine as the free base, was obtained as a yellow to brown oil distillation residue.

### Step B: Preparation of (R)-8-Chloro-1-methyl-2,3,4,5-tetrahydro-1H-3-benzazepine Hemitartrate

The distillation residue from Step A (crude 8-chloro-1-methyl-2,3,4,5-tetrahydro-1H-benzazepine as the free base) was dissolved in acetone (0.400 kg). The resulting solution was drained and weighed to assay the 8-chloro-1-methyl-2,3,4,5-tetrahydro-1H-benzazepine content by HPLC. Results of the assay were used to calculate charges of acetone, L-tartaric acid, and water. The quantities indicated below are typical for achievement of the target 8-chloro-1-methyl-2,3,4,5-tetrahydro-1H-benzazepine:acetone:L-tartaric acid:water mole ratio of 1.00:9.6:0.25:3.6 prior to addition of seed crystals. More acetone (1.415 kg) was added to the reactor and the stirred reactor contents were heated to 47-52° C. To the resulting solution was added a solution of L-tartaric acid (0.1223 kg, 0.815 mol) in purified water (0.211 kg) at a steady rate over 5-15 min. A thin suspension formed during the addition but then redissolved when the mixture temperature was reestablished at 50° C. Hemitartrate seed crystals (0.80 g) were added to the 50° C. solution to achieve cloudiness and to initiate nucleation. Nucleation was allowed to continue for 2-3 h with agitation at 47-52° C. Acetone (0.473 kg) was added to the reactor while the stirred reactor contents were maintained at 50° C. The resulting suspension was cooled to 0-5° C. slowly over 3-5 h. Stirring was continued at 0° C. for another 1-3 h. The resulting white precipitate was collected on a medium-to-fine filter element and then washed with a mixture of acetone (0.900 kg) and purified water (0.054 kg). The enantiomeric excess (ee) of the wet cake was determined.

If the ee was <98%, the wet cake was transferred back into the reactor and reslurried in a mixture of acetone (1.90 kg) and purified water (0.400 kg) at 55-60° C. for 0.5-1 h. If dissolution had not been achieved after one h, then water (approximately 0.160 kg) was added until a clear solution was achieved. The resulting mixture was then cooled to 0-5° C. slowly over 2-3 h. Stirring at 0° C. was continued for another 3-5 h. The resulting white precipitate was collected on a medium-to-fine filter element and then washed with acetone (0.400 kg) at 0-4° C.

The washed solid product (296 g wet) was dried at 60-65° C. under full vacuum for 15-20 hours. The yield of (R)-8-chloro-1-methyl-2,3,4,5-tetrahydro-1H-3-benzazepine hemitartrate, with about 99.7% ee and 7.5 wt. % water content, was 295 g (27.1% based on racemic 2-chloro-N-(4-chlorophenethyl)propan-1-amine hydrochloride and corrected for product water content).

### Step C: Preparation of (R)-8-Chloro-1-methyl-2,3,4,5-tetrahydro-1H-3-benzazepine Hydrochloride Hemihydrate, Form III

To a reactor equipped with overhead agitation and a nitrogen inlet was charged, in the specified order, (R)-8-chloro-1-methyl-2,3,4,5-tetrahydro-1H-3-benzazepine hemitartrate (1.00 kg containing 7.5 wt % water, 1.71 mol), potassium carbonate (0.508 kg, 3.68 moles), ethyl acetate (2.68 kg), and purified water (2.68 kg). The resulting mixture was stirred at 20-25° C. for 30-40 min, and then the phases were allowed to separate over 0.5-1 h. The lower (aqueous) phase was drained to waste disposal. Purified water (2.68 kg) was added to the reactor, and the resulting mixture was vigorously stirred for 10-20 min. The phases were allowed to separate over 1-1.5 h. The lower (aqueous) phase was drained to waste disposal. With the reactor contents at a temperature of 40-45° C., the solvent was removed by vacuum distillation at pressures falling from 153 torr to 46 torr. The residue was cooled to 20-25° C. Ethyl acetate (3.81 kg) was charged to the reactor, and the distillation residue was dissolved with stirring. The water content of the resulting solution was verified by Karl Fischer analysis to be <0.8 wt. %. The solution was filtered through a polishing filter. The reactor was rinsed through the filter with ethyl acetate (2.33 kg) previously verified by Karl Fischer analysis to have <0.05 wt. % water content. Both the solution and rinse filtrates were charged back into the reactor. Purified water (39.9 g) was added to the reactor. The stirred reactor contents were cooled to 0-5° C., and then HCl gas (19.0 g, 0.521 mol) was added while the stirred reactor contents were maintained at 0-5° C. (R)-8-Chloro-1-methyl-2,3,4,5-tetrahydro-1H-3-benzazepine hemihydrate seed crystals (1.33 g) were added to the stirred reactor contents to initiate nucleation at 0-5° C. The remaining HCl gas (107.6 g, 2.95 mol) was charged to the reactor at a steady rate over at least 1.5-2 h while the stirred reactor contents were maintained at 0-5° C. The resulting suspension was stirred at 0-5° C. for 2 h. The resulting white precipitate was collected on a medium-to-fine filter element. The reactor and then the filtered solid product were washed with ethyl acetate (1.33 kg). The wet cake (ca. 867 g) was dried at full vacuum and 33-37° C. for 20 h or until the cake temperature had been stable for 4 hours, whichever occurred first. The resulting (R)-8-chloro-1-methyl-2,3,4,5-tetrahydro-1H-3-benzazepine hydrochloride hemihydrate (3.7 wt. % water content, 14.7% chloride content, <0.01% ROI, >99.6% ee, >99% HPLC purity, and <0.1% wrong isomer content) was obtained in a yield of about 741 g (89.9%).

Those skilled in the art will recognize that various modifications, additions, substitutions, and variations to the illustrative examples set forth herein can be made without departing from the spirit of the invention and are, therefore, considered within the scope of the invention.

1. A salt selected from:
(R)-8-chloro-1-methyl-2,3,4,5-tetrahydro-1H-3-benzazepine bisulfate salt;
(R)-8-chloro-1-methyl-2,3,4,5-tetrahydro-1H-3-benzazepine hemisulfate salt;
(R)-8-chloro-1-methyl-2,3,4,5-tetrahydro-1H-3-benzazepine mesylate salt;
(R)-8-chloro-1-methyl-2,3,4,5-tetrahydro-1H-3-benzazepine hydrobromide salt;
(R)-8-chloro-1-methyl-2,3,4,5-tetrahydro-1H-3-benzazepine nitrate salt;
(R)-8-chloro-1-methyl-2,3,4,5-tetrahydro-1H-3-benzazepine sesqui-oxalate salt-cocrystal;
(R)-8-chloro-1-methyl-2,3,4,5-tetrahydro-1H-3-benzazepine adipate salt;
(R)-8-chloro-1-methyl-2,3,4,5-tetrahydro-1H-3-benzazepine malonate salt; and
(R)-8-chloro-1-methyl-2,3,4,5-tetrahydro-1H-3-benzazepine hemimalonate salt; and
(R)-8-chloro-1-methyl-2,3,4,5-tetrahydro-1H-3-benzazepine glycolate salt; and
pharmaceutically acceptable solvates and hydrates thereof.

2. The salt according to claim 1, that is (R)-8-chloro-1-methyl-2,3,4,5-tetrahydro-1H-3-benzazepine bisulfate salt.

3. The salt according to claim 2, having an X-ray powder diffraction pattern comprising peaks, in terms of 2θ, at about 5.27°, about 18.05°, and about 18.71°.

4. The salt according to claim 1, that is (R)-8-chloro-1-methyl-2,3,4,5-tetrahydro-1H-3-benzazepine hemisulfate salt hydrate.

5. The salt according to claim 4, having an X-ray powder diffraction pattern comprising peaks, in terms of 20, at about 17.10°, about 20.83°, and about 23.43°.

6. The salt according to claim 1, that is (R)-8-chloro-1-methyl-2,3,4,5-tetrahydro-1H-3-benzazepine mesylate salt.

7. The salt according to claim 6, having an X-ray powder diffraction pattern comprising peaks, in terms of 20, at about 12.95°, about 21.22°, and about 6.51°.

8. The salt according to claim 1, that is (R)-8-chloro-1-methyl-2,3,4,5-tetrahydro-1H-3-benzazepine hydrobromide salt hemihydrate.

9. The salt according to claim 8, having an X-ray powder diffraction pattern comprising peaks, in terms of 20, at about 19.77°, about 23.82°, and about 22.54°.

10. The salt according to claim 1, that is (R)-8-chloro-1-methyl-2,3,4,5-tetrahydro-1H-3-benzazepine nitrate salt.

11. The salt according to claim 10, having an X-ray powder diffraction pattern comprising peaks, in terms of 2θ, at about 5.75°, about 10.28°, and about 13.10°.

12. The salt according to claim 1, that is (R)-8-chloro-1-methyl-2,3,4,5-tetrahydro-1H-3-benzazepine sesqui-oxalate salt-cocrystal.

13. The salt according to claim 12, having an X-ray powder diffraction pattern comprising peaks, in terms of 20, at about 13.52°, about 23.50°, and about 13.31°.

14. The salt according to claim 1, that is (R)-8-chloro-1-methyl-2,3,4,5-tetrahydro-1H-3-benzazepine adipate salt.

15. The salt according to claim 14, having an In some embodiments, the salt has an X-ray powder diffraction pattern comprising peaks, in terms of 20, at about 13.63°, about 23.60°, and about 19.49°.

16. The salt according to claim 1, that is (R)-8-chloro-1-methyl-2,3,4,5-tetrahydro-1H-3-benzazepine malonate salt.

17. The salt according to claim 16, having an X-ray powder diffraction pattern comprising peaks, in terms of 20, at about 17.14°, about 22.08°, and about 16.02°.

18. The salt according to claim 1, that is (R)-8-chloro-1-methyl-2,3,4,5-tetrahydro-1H-3-benzazepine hemimalonate salt.

19. The salt according to claim 18, having an X-ray powder diffraction pattern comprising peaks, in terms of 20, at about 17.90°, about 25.37°, and about 21.81°.

20. The salt according to claim 1, that is (R)-8-chloro-1-methyl-2,3,4,5-tetrahydro-1H-3-benzazepine glycolate salt.

21. The salt according to claim 20, having an X-ray powder diffraction pattern comprising peaks, in terms of 2θ, at about 16.67°, about 22.25°, and about 22.01°.

22. A pharmaceutical composition comprising a salt according to claim 1, and a pharmaceutically acceptable carrier.

23. A process for preparing a pharmaceutical composition comprising admixing a salt according to claim 1, and a pharmaceutically acceptable carrier.

24. A dosage form comprising a therapeutically effective amount of a salt selected from: a pharmaceutically acceptable salt of (R)-8-chloro-1-methyl-2,3,4,5-tetrahydro-1H-3-benzazepine and pharmaceutically acceptable solvates and hydrates thereof, wherein said dosage form is a fast-dissolve dosage form.

25. The dosage form according to claim 24, wherein said salt has an aqueous solubility of:
at least about 400 mg/mL at about room temperature;
at least about 500 mg/mL at about room temperature;
at least about 600 mg/mL at about room temperature;
at least about 700 mg/mL at about room temperature;
at least about 800 mg/mL at about room temperature;
at least about 900 mg/mL at about room temperature; or
at least about 1000 mg/mL at about room temperature.

26. The dosage form according to claim 24, comprising (R)-8-chloro-1-methyl-2,3,4,5-tetrahydro-1H-3-benzazepine hydrochloride hemihydrate.

27. A dosage form comprising a therapeutically effective amount of a salt according to claim 1.

28. A method for weight management, comprising administering to an individual in need thereof, a therapeutically effective amount of a salt according to claim 1.

29. The method according to claim 28, wherein said weight management comprises one or more of: weight loss, maintenance of weight loss, decreased food consumption, increasing meal-related satiety, reducing pre-meal hunger, and reducing intra-meal food intake.

30. The method according to claim 28, as an adjunct to diet and exercise.

31. The method according to claim 28, wherein said individual in need of weight management is selected from:
an obese patient with an initial body mass index >30 kg/m²;
an overweight patient with an initial body mass index >27 kg/m² in the presence of at least one weight related comorbid condition; and
an overweight patient with an initial body mass index >27 kg/m² in the presence of at least one weight related comorbid condition; wherein said weight related co-morbid condition is selected from: hypertension, dyslipidemia, cardiovascular disease, glucose intolerance, and sleep apnea.

32. The method according to claim 28, further comprising administering a second anti-obesity agent to said individual.

33. The method according to claim 32, wherein said second anti-obesity agent is selected from: chlorphentermine, clortermine, phenpentermine, and phentermine, and
pharmaceutically acceptable salts, solvates, and hydrates thereof.

34. The method according to claim 28, further comprising administering an anti-diabetes agent to said individual.

35. The method according to claim 34, wherein said anti-diabetes agent is metformin.

36.-50. (canceled)
